(19) United States
(12) Patent Application Publication
(10) Pub. No.: US 2010/0121420 A1
(43) Pub. Date: May 13, 2010

(54) UVA1-LED PHOTOTHERAPY DEVICE AND METHOD

(76) Inventors: Peter Depew Fiset, Loudonville, NY (US); Christopher Macomber, Albany, NY (US)

Correspondence Address: FOLEY AND LARDNER LLP;SUITE 500, 3000 K STREET NW, WASHINGTON, DC, 20007, US

(21) Appl. No.: 12/607,446

(22) Filed: Oct. 28, 2009

Related U.S. Application Data
(63) Continuation-in-part of application No. PCT/US2008/005469, filed on Apr. 29, 2008.
(60) Provisional application No. 61/193,120, filed on Oct. 30, 2008.
(60) Provisional application No. 60/924,097, filed on Apr. 30, 2007.
(60) Provisional application No. 61/002,649, filed on Nov. 10, 2007.
(60) Provisional application No. 61/003,166, filed on Nov. 15, 2007.
(60) Provisional application No. 61/068,052, filed on Mar. 3, 2008.

(51) Int. Cl. A61N 5/06

(52) U.S. Cl. 607/94; 607/88

(57) ABSTRACT

The present application pertains to UVA-1 LED phototherapy for the treatment of various diseases.

### CROSS-REFERENCE TO RELATED PATENT APPLICATIONS

This application claims priority from U.S. Provisional Application No. 61/193,120, filed Oct. 30, 2008, and is a Continuation-in-Part of PCT/US2008/005469, filed Apr. 29, 2008, which claims priority from provisional application Nos. 60/924,097, filed Apr. 30, 2007; 61/003,166, filed Nov. 15, 2007; 61/068,052, filed Mar. 3, 2008; and 61/002,649, filed Nov. 10, 2007. All of the aforementioned applications are incorporated by reference herein in their entirety.

### BACKGROUND OF THE INVENTION

The present invention relates to phototherapies, including, but not limited to, UV phototherapies, and UV photodynamic therapies. Wherein said UV phototherapy incorporates at least one of a UV source. Wherein said UV source is a combination of one or more components including, but not limited to, a UV-LED, a UVA-LED, a UVA1-LED, and a UVA1C-LED. Phototherapies incorporating photodynamic chemicals are also known as photodynamic therapies. An example of a UVA phototherapy that incorporates a photodynamic chemical is the PUVA photodynamic therapy that incorporates the photodynamic chemical psoralen. Wherein UV phototherapies incorporate controlled UV sources to provide method of therapeutic application of light including, but not limited to, UV, incorporating light emitting devices operating in a range of light suited for a phototherapy including, but not limited to, UV, UVB, UVA, UVA1, and UVA1C. The wavelength of a photon is dependent on the properties of the photon medium including, but not limited to, chemical composition, temperature, and pressure. The refractive index of a given photon medium is a measure of the ratio of wavelength of a given photon in said given photon medium relative to the wavelength of said given photon in a vacuum. Useful phototherapy capabilities include, but are not limited to, the useful emitted light over a continuous non-discrete frequency range (“wavelength range”), the useful emitted light over two or more frequency ranges (“wavelength ranges”), the useful emitted light at a discrete frequency (“wavelength”), the useful emitted light at combinations of one or more discrete frequencies (“wavelengths”), the useful therapeutic action spectra, and the phototherapy system accuracy. Wherein said therapeutic action spectra is defined as the relative therapeutic effectiveness versus wavelength over the wavelength range of interest. Wherein said phototherapy system accuracy is a measure of the difference between the phototherapy prescription and the actual delivered phototherapy using measurement variables including, but not limited to, wavelengths, timing, exposure control, wavelength range accuracy, wavelengths accuracy, timing accuracy, and patient identification accuracy. The aggregation of any useful wavelength and of any useful wavelength range emitted from a lamp is known as the spectral radiance. Spectral radiance is directly related spectral irradiance when the spectral radiance is incorporated as a directed application to a two-dimensional plane of a three-dimensional phototherapy target. Spectral irradiance that varies over time in response to a phototherapy control signal is referred to as “dynamic spectral irradiance”. Spectral irradiance is described as a chart of the photon flux as a function of wavelength. Relative intensity is a measure of the relative power compared to the peak power over the wavelength range of interest. The spectral power is the radiant energy emitted from a device over a period of time for a specific spectral irradiance. Spectral irradiance has implied characteristics including, but not limited to, flux, polarity, and coherence. Relative spectral irradiance has normalized units of flux.

Specific phototherapies treat specific diseases. For example, a specific phototherapy that treats Psoriasis is known as a Psoriasis phototherapy. Another example of a specific phototherapy is the FDA approved therapy for Cutaneous T-Cell Lymphoma known as Cutaneous T-Cell Lymphoma extracorporeal photophoresis. Finally, many other phototherapies treating other specific diseases including, but not limited to, moderate to severe acne, newborn hyperbilirubinemia, dermatitis, and Vitiligo are described in the body of this document. Some of the specific phototherapies described in this document are known in the prior art. In addition to the prior art phototherapies described in the body of this document, novel phototherapy means and methods are disclosed herein. Phototherapy capabilities are dependent on the characteristics of the phototherapy components including, but not limited to, the incorporated light emitting devices (“lamps”). Wherein said incorporated light emitting devices are combinations of one or more light emitting device types including, but not limited to, high-pressure mercury vapor type, low-pressure mercury vapor type, low-pressure mercury vapor phosphor converted type, incandescent type, and LED type. Improvements to known phototherapies are dependent upon improvements to phototherapy capabilities and components including, but not limited to, lamps, optical spectral filters, the capability to tune the phototherapy spectral irradiance, the capability to modulate the spectral power, the capability to widen the spectral irradiance, the capability to restrict the spectral irradiance of the device, and the capability to dynamically modulate the spectral irradiance of the lamp. Additional phototherapy capabilities include, but are not limited to, at least one of a directed application. Wherein said directed application is dependent on factors including, but not limited to, spatial orientation of the light emitting device relative to the body, and spatial orientation relative to one or more components of the body. The method of tuning the phototherapy spectral irradiance incorporates combinations of one or more methods including, but not limited to, the method of incorporating combinations of one or more LEDs that more closely match a phototherapy prescription, the method of narrowing the spectral irradiance, the method of broadening the spectral irradiance, and the method of aggregation of spectral irradiance of multiple sources.

### Immune System Background Relating To The Embodiments Of Invention

Many pathological disease processes within the human body are controlled, stimulated, modulated or driven by the human immune system. Many pathological disease processes within a given species bodies are controlled, stimulated, modulated or driven by the given species immune system. The immune system is a complex distributed organ system that constantly monitors and protects the body from organisms and substances including, but not limited to, bacteria, viruses, parasites, fungi, particles, chemicals, and any composition of matter recognized by the immune system. The source of said composition of matter recognized by the immune system may be combinations of sources including a non-self source, and a self source. The immune system is comprised of combinations of components including, but not limited to, the bone marrow, the thymus, the lymph nodes, the lymphatics, the mucosal surfaces, all of the cells, and all of the cells derivatives that are involved in the monitoring and functional aspects of the immune system. The term “recognized” when used to describe an action of the immune system, is descriptive of a process whereby a specific antigen and the specific corresponding matching antibody interact to trigger an immune system response (“immune response”). A specific antibody and matching antigen pair are analogous to a lock and a key mechanism wherein the lock is a counter-part to the key and the key is a counter-part to the lock. Wherein said antibody is similar to a lock, and an antigen is similar to a key. In analogous mechanisms, keys of a specific type only match locks of specific types, and antigens of a specific type only matches antibody of a specific type. The immune system response is also known as the immune response. The immune response is triggered by what is known as an antigen. An antigen is any substance that binds to an antibody. An antibody is an antigen receptor component produced by B-lymphocytes and generally found on the surface of a B-lymphocyte (“B-cell”), or found free-floating within body components including, but not limited to, blood, serum, mucosal surfaces, plasma components and fluid compartments. Lymphocytes are cellular components of the immune system and are generally categorized as either a T-lymphocyte (“T-cell”) or as a B-lymphocyte. Antigens are generally foreign to self, but in dysfunctional immune systems antigens can be native to self. Foreign antigens originate from a source external to self Foreign antigens are transferred to self. Typical antigens are foreign antigens. In certain categories of diseases including, but not limited to, the autoimmune disease category, one or more components of self are recognized by the immune system triggering an undesirable immune response. Said undesirable immune response is also known as a dysfunctional immune response. There are four major categories of diseases caused by dysfunctional immune response, these disease categories are the autoimmune disease category, immunodeficiency disease category, allergies and asthma disease category, and transplant disease category.

Said autoimmune disease category, represents diseases including, but not limited to, Systemic Lupus Erythematosus (“SLE”).

Said immunodeficiency disease category, represents diseases including, but not limited to, Acquired Immunodeficiency Disease Syndrome (“AIDS”).

Said allergies and asthma disease category, represents diseases including, but not limited to, Pollen Allergy, and Pollen Induced Asthma.

Said transplant disease category, represents diseases including, but not limited to, Graft Rejection, and Graft-Versus-Host Disease (“GVHD”).

The immune system is a distributed complex collection of cells, chemicals and organs that work cooperatively to provide two levels of protection. The first level of protection is known as innate immunity. The second level of protection is known as adaptive immunity.

Innate immunity is the capability the immune system has starting from conception to be able to recognize and respond to antigens that the species has adapted over time due to eons of natural selection processes Innate immunity is determined by the genetics of the individual within a given species. Innate immune system capabilities change with age Innate immunity is built-in to the individual's genetic code and functioning prior to birth and after birth Innate immunity is the first line of defense against potential infection from a limited set of antigen types Innate immunity is comprised of immune system components including, but not limited to, phagocytic cells, physical barriers, and any specific molecules capable of recognizing specific antigens. Wherein said physical barriers include combinations of one or more barriers including, but not limited to, skin, and cell membranes. An innate immune response typically occurs within a few hours, has a fixed and limited specificity, and provides a substantially identical immune response to re-infection by the same antigen source.

Adaptive immunity allows the body to survive encounters with antigens that the body has either not encountered before, has lost previous adaptive immunity capabilities, or that the body has no innate immunity against. Adaptive immunity protects the body by providing the body with the capability to substantially increase sensitivity to previously recognized specific antigens. Specific antigens have combinations of one or more, antigen sources including, but not limited to, specific foreign pathogens, and self. Specific pathogens are combinations of one or more pathogens including, but not limited to, bacteria, viruses, parasites, and fungi. Adaptive immunity provides the body with the capability to enhance the effectiveness of an immune response to previously encountered infections. Adaptive immunity provides the body with the capability to protect the body from the previously encountered specific antigen the next time the body encounters the previously encountered specific antigen. The capabilities of the adaptive immune system provide the immune system with the capability to learn and adapt over time as the body interacts with antigen sources including, but not limited to, the surrounding environment, and self. The normally functioning adaptive immune system has four specific immunologic properties: diversity, memory, specificity, and self versus non-self recognition. The memory property of the normal adaptive immune system is based on the higher concentration of lymphocytes with the specific antibody matching the previously encountered antigens created during an initial primary immune response. The adaptive immune system typically requires 5-6 days, to generate an initial primary immune response. The adaptive immune system has highly diverse specificity that improves as the immune response progresses. Upon re-infection by the same antigen source, the secondary immune response by the adaptive immune system is generally more rapid than the primary immune response. The immune system components involved in the adaptive immune response include, but are not limited to, the lymphocytes, the antibodies, antigen presenting cells, and the antigen-specific receptors.

In a normally functioning immune system, the innate immune response and the adaptive immune response work together in a synergistic manner, providing an immune response that is considerably more effective than either the innate immune response or the adaptive immune response could provide without the other. The immune response is moderated by a variety of immune system signaling mechanisms including, but not limited to, chemicals, and other molecules. The immune system signaling mechanisms communicate immune system states and immune system events between cells, compartments and other tissues. The immune system signaling mechanism operates throughout the course of both normal processes and pathological processes.

### Autoimmune Disorders

For people without autoimmune diseases, there are combinations of one or more self-protection mechanisms functioning within the body to prevent the immune system from the dysfunctional condition of recognizing self as foreign. However, for people with at least one autoimmune disease and for people with diseases having pathological processes similar to autoimmune diseases, one or more of the self-protective mechanisms may be impaired due to combinations of one or more impairment mechanisms including, but not limited to, genetic impairment, impairment from injury, and impairment due to immunological changes brought on by the environment. Wherein said autoimmune disease is a disease category including, but not limited to, SLE. Autoimmune diseases resulting from one or more impaired self-protective mechanisms include, but are not limited to, SLE, Hashimoto's Thyroiditis, Graves Disease and Ankylosing Sponylitis. In an autoimmune disorder, the body's immune system generates an immune response against components of self In the autoimmune disease process the immune system mistakes self for non-self. The symptoms and manifestations of a specific autoimmune disease depend on the specific tissue(s) and on the specific organ(s) being attacked by the immune system. The symptoms and manifestations of a specific autoimmune disease can either appear localized, appear systemically, or any combination thereof.

### Immunodeficiency

In addition to dysfunctions in the immune system associated with autoimmune disorders, malfunctions in the immune system may also result from either genetic or environmental causes. The Acquired Immunodeficiency Syndrome (“AIDS”) is an example of a malfunctioning immune system resulting from an environmental cause. Wherein said environmental cause of AIDS is the Human Immunodeficiency Virus (“HIV”). The physiological condition resulting from a malfunction of any component of the innate or adaptive immune system is called immunodeficiency. The physical symptoms and manifestations of immunodeficiency depend on the specific component(s) of the immune system malfunctioning.

### Allergic Reactions

Allergies and allergic disorders including, but not limited to, asthma, are a result of an undesired immune response to combinations of one or more specific particle and/or antigen including, but not limited to, an allergen. Allergies and allergic disorders involve cells including, but not limited to, B-lymphocytes, and mast cells. Allergic reactions result from mast cells releasing allergenic chemicals including, but not limited to, histamine. Atopy is defined as IgA (“immunoglobulin A”) hypersensitivity resulting in asthma, hay fever and other dermatology abnormalities.

### Graft Problems

Graft rejection disease processes are in many ways similar to autoimmune disease processes. An autoimmune disease process including, but not limited to, the process wherein the body's immune system recognizes one or more components of self as foreign. The graft rejection process is a combination of one or more rejection processes including, rejection processes by the host in response to contact with the donor tissue, and rejection processes by the donor tissue in response to contact with the host tissue. In a first graft problem rejection scenario, a graft problem exists if donor tissue recognizes the host tissue is foreign. Said first graft problem rejection scenario is commonly known as GVHD. In a second graft problem rejection scenario, a graft problem exists if a graft recipient tissue recognizes the donor tissue is foreign. Said second graft problem rejection scenario can be further categorized into three time-delineated sub-categories of rejection, acute rejection, hyper-acute rejection, and chronic rejection. Wherein said acute rejection occurs over hours to months. Wherein said hyper-acute rejection occurs within minutes to hours after graft implantation. Wherein said chronic rejection can happen at any point over the life of the host patient. In a third graft problem rejection scenario, a graft problem exists if both the host tissue recognizes the donor tissue is foreign, and the donor tissue recognizes the host tissue is foreign. Donor tissue usually causes the generation of an immune response by the host's immune system, since the donor tissue is generally recognized as foreign (i.e., not an allograft) by the immune system. Wherein said donor tissue is comprised of one or more tissues including, but not limited to, skin grafts, heart transplants, blood transfusions, and any graft of donor tissue(s) to host. When the foreign tissue from a donor is grafted into the host, the host immune system can respond to the graft as if the donor tissue were an invasion of foreign pathogens whereby the host immune response can result in the destruction, or injury, of the grafted donor tissue. Conversely, if the graft tissue has retained donor immune system capabilities, then the graft can recognize the host tissue as foreign and the graft can react against the host tissue with a donor immune response resulting in GVHD.

Down-regulating UV phototherapies including, but not limited to, UVA1 phototherapies, and UVA1C phototherapies, have the capability to down-regulate one or more graft problem rejection scenarios. Said down-regulating UV phototherapies have mechanisms including, but not limited to, promoting the killing, modifying, and/or disabling of activated lymphocytes that are involved in the immune response rejection processes, and promoting the modifying of activated lymphocytes that are involved in the immune response rejection processes. Said down-regulating UV phototherapy targets combinations of one or more target cells including, but not limited to, host lymphocytes, and donor lymphocytes. In addition, said down-regulating UV phototherapies promote increased periods of time before a graft tissue rejects or is rejected by down-regulating the immune system and by down-regulating the cells that promote the immune rejection process. Useful methods of said down-regulating UV phototherapies include, but are not limited to, the useful method of promoting a reduction in the quantity of undesirable drugs that have pharmacological side effects on the patient, and the useful method of improving the patient's quality of life. Said undesirable drugs include, but not limited to, corticosteroids, and immune modulating drugs.

Up-regulation of target cells is a term used to describe phototherapeutic effects including, but not limited to, increasing the activity of target cells, and increasing the population of target cells.

### Cellular and Chemical Components

The generation of effective immune responses requires two categories of cell types; the first category is the lymphocyte cell types, and the second category is the antigen-presenting cell types. Lymphocytes are a sub-category of the white blood cell category. Lymphocytes are produced in the bone marrow by hematopoiesis. The lymphocytes are released from the marrow, and then the lymphocytes circulate through the lymphatic system, circulate through the blood, and circulate in specific lymphoid organs including, but not limited to, thymus, and lymph nodes. Lymphocytes produce and display antigen-binding receptors on the lymphocyte cell surface. The antigen-binding receptors on lymphocyte cells surfaces defines and dictates the lymphocytes capabilities including, but not limited to, diversity, specificity, non-self recognition, and memory of the immune system. There are two types of lymphocytes, the T-lymphocyte types and the B-lymphocyte types.

### B-Lymphocyte

The B-lymphocytes mature in the bone marrow. Upon maturation the B-lymphocyte is released from the bone marrow. A mature B-lymphocyte expresses the same specific antigen-binding receptor at each of the approximately 100,000 antibody locations on that B-lymphocyte. In the B-lymphocyte maturation process, the specific antigen binding receptor for any given B-lymphocyte is determined during cell mitosis by the gene recombination process and is thereby set to one specific antigen binding receptor from a set of approximately 1 billion potential antigen-binding receptors. Each one of the approximately 100,000 identical antigen-binding receptors on the surface of any given lymphocyte is known as an antibody. Antibodies are a type of receptor on the surface of a lymphocyte that is potentially a matching site for an antigen. An antibody is a potentially matching site for a specific immune system activating antigen. When the mature un-activated B-lymphocyte, often referred to as a naive B-lymphocyte, initially encounters an antigen that matches the naive B-lymphocytes specific antibody, the B-lymphocyte becomes activated. Once activated, a B-lymphocyte is no longer described as naive, but is known as an activated B-lymphocyte. An activated B-lymphocyte divides rapidly by the clonal expansion process. The resulting additional cloned B-lymphocytes are naive B-lymphocytes derived from clonal expansion then differentiate into either memory cells, or into plasma cells. The cells from clonal expansion are composed of memory-B plasma cells and effector-B plasma cells. Plasma cells are capable of secreting soluble antibodies. The secreted soluble antibodies are generally not attached to a B-cell and therefore are free floating and circulate in the blood serum. A memory B-lymphocyte has a longer life than a naive B-lymphocytes. The aggregate set of specific memory B-lymphocytes provides the immune system with the memory capabilities of the immune system for protection over a lengthened period of time from a possible re-infection with specific matching antigens that match at least one of the aggregate set of specific memory B-lymphocytes antibodies. The circulating detached antibodies that are secreted from a B-lymphocyte are responsible for generating, what is referred to as, the humoral immune response. An antibody can be of various types including, but not limited to, the free floating circulating antibody type, and antibodies can be bound to lymphocyte cell surfaces. There are five categories of antibodies known as immunoglobulins (“Ig”) including, but not limited to, IgG, IgA, IgE, IgD, and IgM.

### T-Lymphocytes

T-lymphocytes are initially produced in the bone marrow, then translocate to thymus and subsequently mature in the thymus. T-lymphocytes express a distinctive antigen-binding receptor known as a T-lymphocyte receptor (“TCR”). The two types of T-lymphocytes are the helper type T-lymphocytes (“T*SUB*H”) cells, and the cytotoxic type T-lymphocytes (“T*SUB*C”) cells. The T*SUB*H cells typically express a CD4 glycoprotein marker on the T*SUB*H cell surface, whereas the T*SUB*C cells have CD8 glycoprotein marker on the T*SUB*C cell surface. B-lymphocytes and T-lymphocytes have different activation mechanisms. B-lymphocytes activate when an antibody on the B-lymphocyte cell surface recognizes a matching antigen. T-lymphocytes activate when presented with an antigen that is bound to a specific cell membrane protein known as a major histocompatibility complex (“MHC”). A type of MHC known as an MHC class I, is located on the cell surface of just about all nucleated cells in vertebrates. Another type of MHC known as MHC class II is located only on the surface of antigen-presenting cells. When a T*SUB*H cell is presented and interacts with an antigen bound to an MHC class II molecule, the T*SUB*H cell becomes activated. After a T*SUB*H cell becomes active, the T*SUB*H cell is also known as an activated T*SUB*H cell. An activated, T*SUB*H cell typically secretes cytokines. The T*SUB*H cell secreted cytokines go on to activate cell types including, but not limited to, macrophages, and B-lymphocytes. The T*SUB*H cell secreted cytokines include but are not limited to combinations of one or more cytokines including, but not limited to, IL-2, IL-3, IL-4, IL-5, tumor necrosis factor beta (“TFN-beta”), and gamma-interferon. The activation of immune system cells including, but not limited to, lymphocytes, heralds a metabolic change within the specific T*SUB*H cell. Depending on the specific type of cytokine produced by the T*SUB*H cell, there can be a variety of immune responses that occur and that are unique to many different disease processes. The T*SUB*H cell secretions perform many functions including, but not limited to, the activation of both T*SUB*C and T*SUB*H cells, the activation of macrophages, and the class-switching of B-lymphocytes. Class-switching of a B-lymphocyte occurs when a B-lymphocyte, that normally produces one type of immunoglobulin, begins producing a different type of immunoglobulin following class-switching stimulation. An example of a B-lymphocyte class-switching immune response includes the transformation of the T*SUB*C cell into a cytotoxic T-lymphocyte (“CTL”) that has cytotoxic (“cell-killing”) capabilities upon cells presenting the targeted antigen source.

### Antigen-Presenting Cells

Antigen presenting cells (“APCs”) include, but are not limited to, B-lymphocytes, macrophages, and dendritic cells. APCs have capabilities including, but not limited to, the production of cytokines to activate T*SUB*H cells that express the MHC class II molecules on their surface. In order for the immune system to work properly, T-lymphocyte activation must only occur when a T*SUB*H cell recognizes an antigen that is bound to an MHC class II molecule on the surface of an APC. When the immune system is dysfunctional, the T*SUB*H cells may, or may not, have the capability to recognize self. The dysfunctional immune system case wherein the T*SUB*H cells recognize self leads to an autoimmune disease process where the immune system recognizes one or more components of self as an antigen.

The immune system response is comprised of combinations of one or more responses including, but not limited to, the humoral immune response, and the cell-mediated immune response. The humoral immune response requires that a B-lymphocyte encounter an antigen, after which the B-lymphocyte will differentiate into plasma cell types that secrete soluble antibodies. A secreted antibody promotes the clearance of a matching antigen from the body. The cell-mediated immune response is slightly more complex than the humoral immune response. The cell-mediated immune response requires that certain T-lymphocytes recognize the antigen presented on an APC cell. The T*SUB*H cell responds to the matching antigen by producing cytokines The T*SUB*C cell then transforms into the CTLs, which have the capability to kill the infected cells or any other cell displaying the antigen. The cytokines released from T*SUB*H cells have capabilities including, but not limited to, activate T*SUB*H cells, activate T*SUB*C cells, activate macrophages, and cause class-switching of B-cells. Wherein said class-switching of B-lymphocytes including, but not limited to, the change of an IgG B-cell to an IgE B-cell.

### Antigen Specificity

During the B-lymphocytes maturation process and during the T-lymphocytes maturation process, all of the approximately 100,000 binding regions on the surface of any single lymphocyte are created identical to one single specific antibody pattern from the approximately 1,000,000,000 possible antibody patterns generated from the gene rearrangement process during cell mitosis. The gene rearrangement process allows each lymphocyte cell line to generate approximately one billion different receptors targeting a large number of possible antigens. For each lymphocyte cell line, the gene rearrangement process provides a large diversity of potential antibodies, any one antibody of which is expressed identically approximately 100,000 times on a given lymphocyte cell surface. In a normally functioning immune system, upon maturation, any lymphocyte that has an antibody for the “self” is eliminated through normal cell degradation pathways. The normal cell degradation pathway is a mechanism that normally prevents autoimmunity disease expression. Lymphocyte variations result from the process of gene rearrangement. The gene rearrangement process determines which one of the approximately 1,000,000,000 possible antigen-binding receptors patterns to be the pattern of the approximately 100,000 identical antibody sites that have identical antigen specificity on a given lymphocyte. Each T-lymphocyte has 100,000 identical antibody receptor sites expressed on the cell surface. Through a plurality of possible specific rearrangements of the genes of each of the T-lymphocyte receptors (”TCRs“), the TCRs generated are a variety of over approximately 1,000,000,000 distinct antigen receptors. The gene rearrangement process determines the specific antigen pattern for any given T-lymphocyte. Approximately 1 billion antigen patterns are possible as a result of the gene rearrangement process. Said antigen pattern is the pattern that corresponds to the matching antibody pattern.

### Cellular Markers

A cell marker is a cell-specific protein useful for identifying and quantifying the presence of a specific cell type via laboratory tests from the aggregate set of cells in a sample including, but not limited to, the various types of immune cells. Said cell markers are proteins including, but not limited to, certain cell surface proteins. Said cell markers are analyzed during a set of one or more laboratory tests to provide useful cell identification information including, but not limited to, concentration of identified cells. Said set of laboratory tests provide useful methods including, but not limited to, determining patient health, historical patient health records, phototherapy feed-back controls, phototherapy feed-forward controls, phototherapy optimization controls, results of phototherapy treatments, treatment effects, and phototherapy research, specific cell type concentrations. Wherein said set of laboratory tests is comprised of combinations of one or more tests including, but not limited to, flow cytometry.

### Clonal Expansion

The clonal expansion process is a critical process in the generation of an appropriate immune response. The clonal expansion process provides the capability to generate a fast large-scale response to infections and other disease entities. The clonal expansion process is also a critical process leading to many pathological diseases. A lymphocyte has an antigen specificity that is set by gene rearrangement in the bone marrow. As lymphocytes mature, the lymphocyte antigen specificity remains set and the cell then circulates throughout the body. Antigen specificity is determined before the cell ever makes contact with the specific matching antigen of the cell. When a lymphocyte, either a B-lymphocyte or a T-lymphocyte, interacts with the corresponding antigen for that specific matching antibody of the cell, the cell becomes active and then undergoes clonal expansion. Clonal expansion of an activated lymphocyte is a process resulting in the generation of large numbers of cloned lymphocyte cells, wherein all the cloned lymphocyte cells have the same antigenic specificity. Clonal expansion allows the body to mobilize many identical lymphocyte cells against antigens of the same identical antigen type, in order to generate an effective immune response. Some of the cloned cells generated from clonal expansion are memory-B plasma cells with longer lives. Memory-B plasma cells provide a framework for the immune system to respond to future infections by the same antigen type to be recognized sooner than would occur without the memory-B plasma cells. A primary infection occurs when there are substantially no memory-B plasma cells present. A primary infection is typically the first infection of any specific antigen type. A re-infection of the same antigen in the presence of a statistically significant quantity of matching memory-B plasma cells type is known as a secondary infection. A secondary immune response is an immune response to a secondary infection. A secondary immune response has characteristics including, but not limited to, faster response, and heightened immune response. The secondary immune response characteristics result from factors including, but not limited to, the presence of a higher concentration of target specific memory-B plasma cells. The higher concentration of target specific lymphocyte cells present during a primary immune response creates the conditions that have a higher probability that a re-infection by the same antigen will be detected faster. Secondary immune responses tend to be activated faster due to a higher lymphocyte concentration than would have existed without the primary infection. The immune response characteristics that distinguish a secondary immune response from a primary immune response resulting from the primary immune response are characteristics including, but not limited to, the presence of a significantly higher concentration of antigen specific lymphocytes than are present prior to a primary infection. Said antigen specific lymphocytes include, but is not limited to, memory-B plasma cells.

### PRIOR ART

The first pseudo-phototherapy made use of natural sunlight. The ultraviolet wavelengths within sunlight have some beneficial and therapeutic qualities, have some neutral qualities and have some harmful qualities. Historically, UV phototherapy inventions focused on usefulness by reducing the harmful spectral irradiance and/or by providing the beneficial spectral irradiance. The harmful wavelength range(s), the harmful wavelength(s), the beneficial wavelength range(s), and the beneficial wavelength(s) depend on the specific disease and/or disease combinations being treated.

### Ultraviolet Light

Solar radiation spans a range of wavelengths including, but not limited to, visible light, infrared light and ultraviolet light. Sunlight is filtered solar-radiation at or near the surface of the Earth. Sunlight is comprised of a combination of wavelength ranges, including, but not limited to, ultraviolet light, visible light and infrared light. The specific spectral irradiance in sunlight is complex and depends on dynamic sunlight variables including, but not limited to, time of day, cloud cover, solar flares, sun spots, atmospheric composition, time of year, elevation, atmospheric pressure, humidity, water vapor, water solids, water liquids, solids, liquids, vapors, dust, elements, compounds, air-pollution concentrations, and latitude.

In the interest of tanning and UV phototherapy, the concern for improvements and usefulness is focused on the ultraviolet light ranges.

Within UV there are wavelengths that produce combinations of various effects including harmful effects, beneficial effects, and neutral effects. Wherein said various effects are dependent on conditions including, but not limited to, patient exposure history, patient health history, and patient drug history.

After passing through the ozone and other atmospheric absorbers, substantially all UVC solar radiation is absorbed and a majority of the UVB solar radiation is absorbed. The majority of the ultraviolet sunlight reaching the earth is UVA light. The solar radiation reaching the surface of the earth is known as terrestrial sunlight (“sunlight”). The ozone present in the atmosphere blocks most light up to 310 nm but theories have been advanced in the literature indicating that damage to the ozone layer has led to an increase in exposure to UVB light. Thus, in general, as the ozone is further depleted, a good tan becomes more and more useful to protect from harmful sunlight and a tan can be considered a prophylactic for sunlight-induced disease. Specific skin types have varying responses to prophylactic effects of a tan, including a marginal prophylactic effect for people with skin type I. Skin type-I is commonly known as very fair skin that does not tan easily.

### Science and Technology

Scientific studies of the effects of UV radiation exposure have accumulated slowly over the past few decades. In general, scientific knowledge is currently accelerating due to improvements in UV light sources, and measurement technologies and in analysis capabilities. The acceleration of scientific knowledge is expected to also affect scientific knowledge in the effects of UV radiation exposure. Many scientific experiments and studies of UV exposure were handicapped by the limited capabilities of legacy light sources. The legacy light sources used included mercury vapor based lamp technologies including, low-pressure mercury vapor lamps, low-pressure mercury vapor phosphor converted (“fluorescent”) lamps, and high-pressure mercury vapor lamps.

Originally, phototherapies were invented by making controlled use of crude lamps selected from the available lamp types including mercury vapor lamp types, fluorescent lamp types, and high-pressure mercury vapor lamp types. Crude light sources are types of lamps that have little or no control of important phototherapy variables including, but not limited to, spectral irradiance, and dynamic spectral irradiance.

Thereafter, inventors invented specific phototherapies to treat patients with specific diseases. Inventors made useful improvements to known phototherapies incorporating optical spectral filters, reflectors, specialized gas discharge lamps, and phosphor converters. Useful improvements included improvements that made the phototherapy more effective, and reduced undesirable side effects. As an example, the photophoresis phototherapy further incorporated improvements including, sterilized tubes, sterilized pumps, and sterilization means. A given specific phototherapy is specialized to treat specific disease indications.

Many prior art phototherapies contain undesirable quantities of UVB and UVC depending on the characteristics of the phototherapy device. Thus, if the prior art phototherapies are used, patients run a significant risk of exposure to harmful wavelengths of light as the current delivery methods are unable to be tuned to provide the most effective spectral irradiance for many phototherapies including, but not limited to, the target diseases described herein. The use of certain prior art phototherapies has tremendously negative implications for patients with photosensitive diseases including, but not limited to, SLE, xeroderma pigmentosum and vitiligo. Prior art phototherapies make use of legacy light sources that not only have decreased total output, but more specifically a shift in the spectral irradiance. The spectral irradiance is the most important characteristic of a phototherapy and an uncontrolled shifting in spectral irradiance does not benefit the phototherapy patient. In addition, because of the inability for the lamps incorporated in the aforementioned prior art phototherapies to maintain a constant spectral irradiance output of the required light over time, patients run an increased risk of exposure to non-prescribed potentially damaging light as the prior art devices degrade over time. Certain prior art phototherapies that prescribe UVA light could be improved upon further by reducing the amount of UVB and UVC in the spectral irradiance. Certain prior art phototherapies that prescribe UVA light have an undesirable spectral irradiance of stray light within the UVB or UVC range. The UVB and UVC light increases the likelihood of the development of skin cancers such as basal cell carcinoma, squamous cell carcinoma, and melanoma. Therefore patients using prior art phototherapies are subjected to the uncertainty of light emitting devices that have relatively low reliability and relatively high failure rate over time. Moreover, patients using prior art phototherapies are not getting the useful benefits of a light source that allows the phototherapy prescription specifications of the more beneficial wavelengths of light over those that are considered more detrimental. It is expected that the effectiveness of phototherapy prescriptions will improve over time in ways including, but not limited to, patient-by-patient tuning of phototherapy prescriptions, per session tuning of phototherapy prescription specifications, per session tuning of spatial resolution, finer resolution of spectral irradiance specifications, and fine tuning dynamic spectral irradiance specifications.

Although many prior art phototherapies are relatively mature and may have reasonable costs and are generally available and are useful for mitigating specific diseases, the prior art phototherapies do not offer suitable or adequate phototherapies for diseases including, but not limited to, said target disease, SLE, Ulcerative Colitis, and Crohn's Disease. The prior art phototherapies tend to have fixed discrete spectral lines that in general are not optimized for any phototherapy.

Several types of phototherapies have been proposed in the prior art. A few of the classification properties are in general use on skin, use in skin, use within a body cavity, use within a lumen, use on tissue, use within tissue, use on tissue within the cavity of a human or that of an animal, any specific type of cell in an animal, use within blood, and use on the blood. Wherein the use of a phototherapy to treat the blood is made through the skin and dermal layers into surface capillaries based on the penetration capabilities of the light, made within a cavity, lumen or a blood vessel of any type.

All the phototherapies heretofore known suffer from a number of disadvantages. The combination of the aforementioned prior art suffers from an inability to solve basic problems including, but not limited to, stray UVB and UVC, rough spectral irradiance, poor phototherapeutic prescription accuracy of delivery, and limited dynamic spectral irradiance control. Even when combined into unique devices or treatment modalities the prior art suffers from many disadvantages. The fundamental disadvantage of the prior art phototherapies including, but not limited to, inconsistent directed application of a specific, narrow-band wavelength light that is optimized for abandoned phototherapy prescriptions. The prior art light sources have limited customization capabilities and represent a significant risk of exposure to stray UVB and/or UVC light for the methods of UVA phototherapies, UVA1 phototherapies, and UVA1C phototherapies. The prior art phototherapies have the disadvantage of emitting significant amounts of fixed discrete spectral lines that have the negative effect of potentially saturating the set of chromophores capable of absorbing the discrete wavelength thereby overloading the phototherapeutic action at non-optimized discrete wavelengths.

### BRIEF DESCRIPTION OF THE DRAWINGS

FIG. 1 lists the irradiance range for different wavelengths of light emitted by the LED's.

FIG. 2 shows a phase-contrast micrograph of a representative field of cells in culture 1 day after plating. Neurons meeting the criteria for stage 1-3 were included in the quantitative analyses. Stage 1 neurons are encircled by lamellipodia, stage 2 neurons have 2-5 processes ≦20 μm in length, stage 3 neurons have 2-5 processes and an axon ≧40 μm in length that is also at least 20 μm longer than the next longest process. Non-neuronal and dying cells were excluded (boxed).

FIG. 3 shows the effect of continuous exposure to LED's on apoptosis (at various intensities or wavelengths) for 24 hours beginning at 3 hours after plating.

FIG. 4 shows the effect of continuous exposure to LED in Experiment 8 pod, for 1 day. LED exposure coincident with neuronal development increases the rate of initial polarization and axonogenesis.

FIG. 5 shows number of neurons per field after 18-24 h of exposure to UVA1 LEDs, beginning 3 h after plating. Data are the Mean (±SEM) number of neurons per field in control (Cont) and LED-exposed cultures expressed as a percent of control, and are the combined results of four separate experiments (n=50-79 fields per treatment group). Pods had a single LED emitting one peak wavelength of light (in parentheses) and are plotted in order of increasing irradiance. The range of total energy of session fell between 0.00782 J/cm/cm (for the Pod emitting 0.09 μW/cm/cm) and 24.7 J/cm/cm (for the Pod emitting 286 μW/cm/cm).

FIG. 6 shows the percent of neurons with axons per field after 18-24 h exposure to UVA1 LEDs, beginning 3 h after plating. Data are the Mean (±SEM) percent of neurons with axons per field in control (Cont) and LED-exposed cultures, expressed as a percent of control, and are the combined results of four separate experiments (n=50-79 fields per treatment group). Pods each had a single LED emitting one peak wavelength of light (in parentheses) and are plotted in order of increasing irradiance. The range of total energy of session fell between 0.00782 J/cm/cm (for the Pod emitting 0.09 μW/cm/cm) and 24.7 J/cm/cm (for the Pod emitting 286 μW/cm/cm).

FIG. 7 shows the number of neurons per field after 18-24 hr exposure to UVA1 LEDs, beginning 1 d after plating. Data are the Mean (±SEM) percent of neurons with axons per field in control (Cont) and LED-exposed cultures, expressed as a percent of control, and are the combined results of four separate experiments (n=42-57 fields per treatment group). Pods each had a single LED emitting one peak wavelength of light (in parentheses) and are shown in order of increasing irradiance. The range of total energy of session fell between 0.00782 J/cm/cm (for the Pod emitting 0.09 μW/cm/cm) and 24.7 J/cm/cm (for the Pod emitting 286 μW/cm/cm).

### DETAILED DESCRIPTION

### Definitions and Acronyms

A light emitting diode (“LED”) is a combination of one or more LED components including, but not limited to, an electroluminescent semiconductor material, a substantially translucent package, an electric current conductor, an electric voltage insulator, a photon emission path, a power supply, a LED heat sink, an array of electroluminescent semiconductor material, an addressable array of electroluminescent semiconductor material, a bonding material, a wire-bonding material, an LED component fastener, and a LED integrated circuit. Said photon emission path is comprised of one or more combinations of photon emission path components including, but not limited to, a translucent optical lens, and a wavelength dependent photonic filter. LEDs are selected from one or more LED types including, but not limited to, laser diode, organic light emitting devices, periodic table of the elements group IV based semiconductor LED type, periodic table of the elements group III-V based semiconductor LED type, periodic table of the elements group II-VI based semiconductor LED type, optically pumped LED type, electrically pumped LED type, and organic light emitting device type. Wherein said organic light emitting device type includes, but is not limited to, organic LED type. Wherein said periodic table of the elements group IV based LED type including, but not limited to, germanium based LED type, silicon based LED type, carbon nanotube based LED type, silicon carbide (“SiC”), and diamond based LED type. Wherein said periodic table of the elements group III-V LED type including, but not limited to, aluminum nitride (“AlN”) based LED type, gallium nitride (“GaN”) based LED type, aluminum phosphide (“AlP”) based LED type, gallium phosphide (“GaP”) based LED type, aluminum selenide (“AlSe”) based LED type, gallium selenide (“GaSe”) based LED type, and aluminum indium gallium nitride (“AlInGaN”) based LED type. Wherein said periodic table of the elements group II-VI LED type including, but not limited to, zinc oxide based LED type, zinc sulfide based LED type, zinc selenide based LED type, zinc telluride based LED type, cadmium oxide based LED type, cadmium sulfide based LED type, cadmium selenide based LED type, cadmium telluride based LED type. The stochastic ratio of elements in said LED and in other compound semiconductors varies considerably; and it is understood that the exact stochastic ratios of elements and doping processes are well-know in the literature and do not need to be re-iterated herein.

The wavelength of a photon is specified herein relative to a vacuum.

The term “UV” represents any photon with wavelength less than or equal to 400 nanometers.

The term “UVA” represents any photon with wavelength greater than 290 nanometers and less than or equal to 400 nanometers, and includes a subrange of 320 to 400 nanometers.

The term UVA1 is equivalent to the terms UV-A1, and UVA-1. The term “UVA1” represents any photon with wavelength greater than 340 nanometers and less than or equal to 400 nanometers. The term UVA1 is equivalent to the terms UV-A1, and UVA-1.

The term “UVA1C” represents any photon with wavelength greater than 340 nanometers and less than or equal to 415 nanometers. The term UVA1C is equivalent to the terms UV-A1C, UVA-1C, and UVA1-C.

The term “UVA2” represents any photon with wavelength greater than about 315 nanometers and less than or equal to 340 nanometers. The term UVA2 is equivalent to the term UV-A2, and UVA-2.

The term “UVA3” represents any photon with wavelength greater than about 290 nanometers and less than or equal to 315 nanometers. The term UVA3 is equivalent to the term UV-A3, and UVA-3.

The term “UVB” represents any photon with wavelength greater than 260 nanometers and less than or equal to about 290 nanometers. The term UVB is equivalent to the term UV-B.

The term “UVC” represents any photon with wavelength greater than about 160 nanometers and less than or equal to 260 nanometers. The term UVC is equivalent to the term UV-C.

The term “UV-LED” represents an LED capable of substantial UV electroluminescent emission.

The term “UVA-LED” represents an LED capable of substantial UVA electroluminescent emission.

The term “UVA1-LED” represents an LED capable of substantial UVA1 electroluminescent emission.

The term “UVA1C-LED” represents an LED capable of substantial UVA1C electroluminescent emission.

The term “UVB-LED” represents an LED capable of substantial UVB electroluminescent emission.

The term “UVC-LED” represents an LED capable of substantial UVC electroluminescent emission.

The definition of the term “self” is understood herein to be any tissue native to the body.

The definition of the term “non-self” is understood to be any tissue not native to the body, including, but not limited to, transplanted organs, graft tissue, and transfused blood.

The term “population” when used in the context of cells is understood to represent cell descriptions including, but not limited to, cell concentrations, cell types, cell sub-types, cell state, cell attributes, and cell conditions.

The term “blood vessel” is to mean a combination of one or more blood flow conduits including, but not limited to, a stent, a vein, an artery, a venules, and an arterioles.

The term “spectral irradiance” herein is understood to have units of watt per square meter per nanometer. The term “spectral radiance” is understood to have units of watt per steradian per square meter. The term “relative spectral irradiance” is the spectral irradiance as a function of wavelength over a wavelength range divided by the maximum spectral irradiance in the wavelength range. It is understood that phototherapy sessions have an implied time span. The term “phototherapy dose” herein is understood to be the integration of the phototherapy spectral irradiance with the phototherapy continuous wavelength range(s) and with phototherapy session time plus the sum of all spectral irradiance at discrete wavelength(s) multiplied by the phototherapy time. Phototherapy dose has units of Joules per square meter. It is understood the phototherapy sessions have an implied rate of repetition. It is understood that a default phototherapy repetition rate is substantially one day.

### Preferred Embodiments of the Invention

Accordingly, several non-limiting advantages of the invention include, but are not limited to, the targeting of specific cell types and tissues with light of specific dynamic spectral irradiance in specific locations and/or mediums. Wherein said spectral irradiance is selected to provide a combination of one or more useful methods including, but not limited to, improved phototherapeutic effectiveness, reduced hazards, and reduced collateral damage. Wherein said spectral irradiance is selected to be substantially within a wavelength range including, but not limited to, UVA1, and UVA1C.

When referencing a category of target cell types herein, it is to be understood that the preferred embodiments of the present invention affect all related target cell categories and all related target cell sub-categories. There are too many well-known target cell categories and target cell sub-categories to mention completely, and therefore many are not explicitly listed herein. It is to be understood that well-known target cell categories and well-known target cell sub-categories as described in the literature and in the references are included herein. When referencing a category of target disease types herein, it is to be understood that the preferred embodiments of the present invention affect all related target disease categories and all related target disease sub-categories. There are too many well-known target disease categories and target disease sub-categories to mention completely, and therefore many are not explicitly listed herein. It is to be understood that well-known target disease categories and well-known target disease sub-categories as described in the literature and in the references are included herein.

### Mechanisms

The preferred embodiments of the present invention incorporates any suitable means capable of providing the useful method of provisioning of at least one of a target disease specific by a therapeutic directed application of at least one type of a therapeutic electromagnetic radiation (“EMR”) from at least one type of a therapeutic radiation emitting source configured in at least one of a therapeutic radiation emitting source form factor applied to at least one type of a therapeutic radiation targeted body component in order to provide at least one of a desirable therapeutic effect upon said therapeutic radiation targeted body component.

Wherein said therapeutic electromagnetic radiation is comprised of a combination of one or more electromagnetic radiation types including, but not limited to, light, visible light, invisible light, ultraviolet, UVA, UVA1, UVA1C, infrared, radio-frequency, x-ray, and microwave. Wherein said therapeutic electromagnetic radiation is further comprised of a dynamic combination of one or more electromagnetic radiation types including, but not limited to, light, visible light, invisible light, ultraviolet, UVA, UVA1, UVA1C, infrared, radio-frequency, x-ray, and microwave.

Wherein said therapeutic radiation emitting source form factor is comprised of combinations of one or more form factors including, but not limited to, a flexible array of LEDs, fluorescent bulbs, filament based bulbs, an LED lamp, an optical spectral filter, an LED lamp and phosphor converters, and semiconductor nanocrystal photonic converters. Wherein said semiconductor nanocrystal photonic converters including, but not limited to, elemental Group IV based photonic converter, elemental Group III-V based semiconductor photonic converter, elemental Group II-VI based photonic semiconductor photonic converter, zinc oxide based nanocrystal converter, and titanium dioxide based photonic converter, Group IV, Group III-V, and Group II-VI semiconductor compounds. Wherein said semiconductor nanocrystal means has capabilities including, but not limited to, optically pumped photonic conversion, electrically pumped photonic conversion, mechanically pumped photonic conversion, nuclear pumped photonic conversion, and magnetically pumped photonic conversion.

Said optical spectral filter means is comprised of combinations of one or more optical spectral filter components including, but not limited to, optical polarizing spectral filter component, dynamic characteristic spectral filter component, long-pass spectral filter component, band-pass spectral filter component, and low-pass filter component. Said optical spectral filter means incorporates dynamic functions including, but not limited to, dynamic spectral tracking system. Wherein said optical spectral filter means is comprised of a combination of one or more optical spectral filter means capabilities including, but not limited to, low-pass optical spectral filter, band-pass optical spectral filter, and high-pass optical spectral filter. Wherein said band-pass optical spectral filter means is preferred over the high-pass filter for reasons including, but not limited to, elimination of photons with wavelengths outside the prescribed phototherapy range(s), elimination of spurious photons with shorter wavelengths than prescribed by the phototherapy, elimination of non-UVA1 photons when providing UVA1 phototherapy, and elimination of non-UVA1C photons when providing UVA1C phototherapy. Wherein said band-pass optical spectral filter means is preferred over the low-pass filter for reasons including, but not limited to, reduction of photons with wavelengths outside the prescribed phototherapy range(s), reduction of photons with longer wavelengths than prescribed by the phototherapy, reduction of non-UVA1 photons when providing UVA1 phototherapy, and reduction of non-UVA1C photons when providing UVA1C phototherapy. Wherein said band-pass filter is preferred over low-pass filter for reasons including, but not limited to, eliminating photons with wavelengths longer than the phototherapy prescription requires, and reducing the spectral irradiance required to provision a phototherapeutic effect.

Wherein said optical spectral filter has one or more optical filter form factors including, but not limited to, fiberoptic, liquid, flexible solid, and rigid solid, gaseaous, and vapor.

Wherein said therapeutic radiation emitting source is comprised of a combination of one or more dynamically controlled radiation emitting sources including, but not limited to, an LED, an optically converting nanocrystal, a solid-state light source, a vapor light source, a gaseous light source, and a liquid light source.

Wherein said dynamically controlled radiation emitting sources incorporate at least one of a radiation emitting source control means responsive to at least one of a radiation emitting source control signal.

Wherein said radiation emitting control signal is responsive to radiation emitting controller.

Wherein radiation emitting controller is comprised of any suitable means capable of providing the useful methods of controlling the emission of radiation to optimize the approximation of a phototherapy prescription.

Wherein said computer means is comprised of any suitable means comprised of combinations of one or more computer components including, but not limited to, analog computer means, optical computer means, digital computer means, computer memory means, computer communications means, computer program means, digital signal input means, analog signal input means, optical signal input means, digital signal output means, analog signal output means, optical signal output means, digital to analog converter means, analog to digital converter means, optical to analog converter means, analog to optical converter means, and spectrometer means.

Wherein said computer communication means is comprised of any suitable means including, but not limited to, wireless communication means. Wherein said wireless communications means including, but not limited to, radio frequency communications means, and optical communications means. Wherein said radio frequency communications means is comprised of a combination of one or more communication components including, but not limited to, an 802.11 based communication means, a BlueTooth based communications means, and a microwave communication means. Wherein said optical communication means including, but not limited to, an infra-red emitter to detector pair communication means, a free-space optics communications means. Wherein said infra-red emitter to detector pair communication means including, but not limited to, an infra-red emitter to detector pair communication means. Wherein said infra-red emitter to detector pair communication means is comprised of any suitable means including, but not limited to, an IrDA standards compliant means, and a SONET standards compliant, a customized optical communications method.

Wherein said therapeutic radiation targeted body component is selected from the category of body components including, but not limited to, a skin, a body cavity, a lumen, an organ, a tissue, a tissue within the cavity of an animal, a tissue within the cavity of a human, a volume of blood, and a targeted cell in an animal.

Wherein said therapeutic directed application is a combination of one or more directed exposure applications of EMR flux of a certain dynamic spectral irradiance comprised of combinations of one or more directed applications including, but not limited to, an external directed application, an internal directed application, a directed application within the blood, a directed application to the blood, a directed application to at least one of a blood component, a directed application to at least one of a separated blood component, a directed application to the blood through the skin, a directed application through the dermal layers into blood vessels based on the penetration capabilities of the EMR, a directed application within a cavity, a directed application within a lumen, and a directed application within a blood vessel or lumen onto desired tissue. Wherein said directed application through the dermal layers into surface capillaries is based on the penetration capabilities of said therapeutic electromagnetic radiation. Wherein said dermal layers have a set of dermal properties specific to each patient. Said set of dermal properties vary for a given patient as a function of dermal variables including, but not limited to, dermal location on body, thickness, blood flow, and phototherapy history. Said dermal variables are used as inputs to control the phototherapy flux to optimize the close approximation of a phototherapy prescription for a customizable phototherapy dependent on dynamic patient state.

Wherein said penetration capabilities vary over the patient population and are generally categorized by skin types. Additional power control factors exist including, but not limited to, blood ion concentration. Power control factors require the power control means to adjust said control spectral irradiance power signal the rate of EMR flux to compensate for the power control factors and thus provide an optimized approximation to the prescribed phototherapy treatment. An additional power control factor is the amount of pigmentation darkening of the skin in any given patient over the duration of the phototherapeutic treatment, and other sources of pigmentation darkening. The power control factors are measured or estimated to provide the power control input signals in order for the power control means to compensate and provide a prescribed phototherapeutic effect. A therapeutic radiation penetration measurement means is optionally incorporated into the preferred embodiments of the present invention to generate a power control compensation signal means. The preferred embodiments of the present invention incorporates any suitable means capable of providing useful methods including, but not limited to, a method to locate and quantify the presence of phototherapy modifying chemicals, and a method to locally reduce undesirable phototherapy modifying chemicals, and a method to locally increase desirable phototherapy modifying chemicals. Wherein said phototherapy modify chemical are comprised of combinations of one or more chemicals including, but not limited to, sunscreens, sun blocks, UV absorbers, UV converting chemicals, zinc oxide, titanium dioxide.

Wherein said therapeutic directed application to the blood is comprised of combinations of one or more directed application means including, but not limited to, an internal directed application to blood means, and an external directed application means.

Wherein said therapeutic directed application to at least one of said blood component is comprised of combinations of one or more directed application means including, but not limited to, an internal directed application means, and an external directed application means.

Wherein said directed application to at least one of said separated blood component is comprised of combinations of one or more directed application means including, but not limited to, an internal directed application to means, and an external directed application means.

Wherein said desirable therapeutic effect is a combination of one or more effects including, but not limited to, increase populations of at least of one of a desirable target cell type, decrease populations of at least one of an undesirable target cell type, modulate populations of at least one of said target cell type, stimulate populations of said target cell type, enhance populations of said target cell type, kill populations of said target cell type, down-regulate populations of said target cell type, alter populations of said target cell type, increase ratios between a plurality of said target cell type populations, decrease ratios between a plurality of said target cell type populations, modulate ratios between a plurality of said target cell type populations, stimulate ratios between a plurality of said target cell type populations, enhance ratios between a plurality of said target cell type populations, down-regulate ratios between a plurality of said target cell type populations, alter ratios of a plurality of said target cell type populations, reduce concentration of at least one of a target inflammatory chemical, and increase the concentration of at least one of a target protective chemical. Wherein said phototherapeutic effects is provisioned by any suitable means capable of providing useful methods including, but not limited to, direct modification of target chemical, killing portions of target cell type population responsible for producing undesired component of an immune response component, stimulating beneficial target cell type clonal expansion, and stimulating said target cell type that are capable of producing a desired component immune response.

Wherein said desirable target cell types is comprised of combinations of one or more cell types including, but not limited to, T*SUB*H1, T*SUB*H2, naive B-cells, memory-B cells, T*SUB*C cells, and said target cells. Wherein said undesirable target cell types is comprised of combinations of one or more cell types including, but not limited to, said target cells.

Wherein said target inflammatory chemical is selected from chemicals including, but not limited to, an inflammatory cytokine. Wherein, said inflammatory cytokine are comprised of cytokine types including, but not limited to, interferon-gamma (“IFN-gamma”), IL-1, IL-2, IL-5, IL-8, TNF-alpha, and TNF-beta. Wherein the term “IL” refers to interleukin.

Wherein said target protective chemical is selected from chemicals including, but not limited to, a protective cytokine Wherein, said protective cytokine is comprised of cytokine types including, but not limited to, interleukin-4 (“IL-4”).

Wherein said target cell type, are preferably selected from cell types including, but not limited to, T*SUB*H1 cells, and T*SUB*H2 cells.

Wherein said target phototherapy is capable of providing benefits to patients with contributions of one or more corresponding target diseases from a class of phototherapies including, but not limited to, SLE phototherapy, inflammatory reactions phototherapy, and immune system modulation phototherapies.

Various specific cells are implicated in specific disorders, such as the cells of the immune system. In general, the phototherapy-modulated cells remain the same cell type, but have a modulated function. Wherein said modulated function has one or more dynamically changing cell function capabilities, including, but not limited to, accelerated normal function, delayed normal function, accelerated altered function, altered function, and delayed altered function. An example of said delayed altered function is the production and the subsequent release of chemicals including, but not limited to, cytokines Dependent on the phototherapy prescription, a given cell type may be desirable in a first case and undesirable in a second case, whereas other cell types have the same phototherapy status in two different disease indications. The preferred embodiments of the present invention have capabilities including, but not limited to, dynamically provide, and spatially position, the flux of a sequence of wavelengths optimized to approximate a phototherapy prescription. The preferred embodiments of the present invention incorporates any suitable means including, but not limited to, said target cell population control means, capable of providing the method of controlling the concentrations of target cells present within target boy components. Wherein said target body components are comprised of body components including, but not limited to a fluid, tissue, lumen, organ or any other component or part of the body during a specific disease processes. Wherein said specific disease process includes, but is not limited to, a dysfunctional immune reaction. The preferred embodiments of the present invention incorporate any suitable means capable of providing the useful methods to change the course of said target disease. The preferred embodiments of the present invention incorporates any suitable means for providing the useful method of deriving a specific and desirable change including, but not limited to, a therapeutic change. The therapeutic benefits of the preferred embodiments of the present invention are achieved by a light interaction comprised of combinations of one or more interaction including, but not limited to, interactions at a cellular (“light-on-cell”), interaction at the sub-cellular level (“light-on-sub-cell”), interaction with fluorophores, and interaction with chromophores. Wherein said light interaction including, but not limited to, the alteration of the inner-workings and metabolic processes of specific cells, the alteration of the inner-workings and metabolic processes of specific sub-cellular components, mitochondria function, DNA integrity, DNA function, lysosomal function, ribosomal function, protein synthesis, and other organelle functions. Wherein said light interaction is comprised of combinations of one or more interactions including, but not limited to, altering said target cells metabolic profile, altering said target cell capabilities, differentiation of said target cell capabilities, and controlled cell death. Wherein said controlled cell death is a combination of one or more processes including, but not limited to, triggering apoptosis, or triggering necrosis. Wherein said light interaction is a combination of one or more interactions including, but not limited to, altering the type of secretions from a secreting cell, and altering the amount of each type of secretions from said secreting cell. Wherein said light interaction is comprised of a light-on-cell interactions including, but not limited to, the alteration of cellular function of specific target cells, and specific target cells components. Wherein said target cell components are comprised of combinations of one or more components including, but not limited to, mitochondria, and other cell organelle types. Wherein said secreting cell is of a cell type including, but not limited to, said target cell types.

The preferred embodiments of the present invention incorporates any suitable means capable of providing useful methods including, but not limited to, a method to control said target diseases, a method to decrease the population of overpopulated cells, a method to eliminate overpopulated cells, a method to modify overpopulated cells, a method to eliminate harmful effects of said target cells, a method to reduce harmful effects of said target cells, a method to modify the secretions of cytokines, a method to modify activation harmful cells, a method to increase protective cells with protective properties, a method to altering target cell ratios, a method to alter the chemical secretions of target cells, a method to increase the presence of various chemicals that promote certain beneficial processes, a method to inhibit certain pathological processes, and a method to promote beneficial processes. For example, in target diseases, including, but not limited to, SLE disease, wherein said overpopulated cells is a combination of one or more cells including, but not limited to, activated T*SUB*H1 lymphocytes. For example, in target diseases, including, but not limited to, SLE disease, ulcerative colitis, and Crohn's disease, wherein said protective cells is a combination of one or more cells including, but not limited to, T*SUB*H2 lymphocytes.

Many diseases involving the immune system result from immune system dysfunction during the clonal expansion process. During the dysfunctional clonal expansion process the lymphocyte cell that recognizes the specific antigen is cloned at a higher rate of cloning than is beneficial resulting in conditions including, but not limited to, lymphocyte hyper-sensitivity, and lymphocyte hyper-reactivity. The resulting excessive concentrations of cloned lymphocyte cells alter the disease process in a substantially negative fashion depending on the cloned lymphocyte cells type and disease processes. The preferred embodiments of the present invention incorporate any suitable means capable of providing the methods including, but not limited to, reducing detrimental lymphocyte clones, eliminating detrimental lymphocyte clones, increasing relative populations of specific cells involved in the pathological process, adjusting cell type populations, adjusting the ratio of lymphocyte cell populations to as close to a prescribed level as possible, the decrease in populations of activated lymphocytes directed against a part or component of the body where it is beneficial to reduce the numbers of activated lymphocytes to decrease damage and to decrease pathological processes, and reducing the concentration of activated lymphocytes. Reducing the concentration of activated lymphocytes results in processes including, but not limited to, a reduction of the inflammatory reaction, and reduction of pathological processes. Wherein said pathological processes is a combination of one or more process including, but not limited to, damage mediated by tissue-binding auto-antibodies, damage to immune complexes, and damage caused by an activated lymphocyte population activated against the body. In diseases including, but not limited to, SLE, it is possible for the preferred embodiment of the present invention to decrease inflammation and/or cell and tissue damage by effects including, but not limited to, decreasing the number of activated T*SUB*H1 cells that are secreting IFN-gamma, decrease the activation of additional harmful cells, and increase the numbers of protective cells. Wherein said protective cells are comprised of at least one of a protective cell type including, but not limited to, IL-4 secreting T*SUB*H2 cells. The preferred embodiments of the present invention provides useful therapeutic methods including, but not limited to, modifying the ratio of specific cell types. The useful therapeutic methods provided by the preferred embodiments of the present invention provides the benefit of slowing the disease progression and provides useful benefits including, but not limited to, decreasing the symptoms within the patient's body, and decreasing the symptoms within the patient's body components where the disease is having an effect. The preferred embodiments of the present invention incorporate any suitable means capable of providing the useful methods of controlling disease symptoms, whereby a patient will not be required to use as many pharmacological drugs. Wherein said pharmacological drugs are combinations of one or more drugs including, but not limited to, immunomodulating drugs, and steroids. The pharmacological drugs used to treat diseases including, but not limited to, SLE, typically can't be used for long periods of time, since the pharmaceutical drugs produce many serious side effects including, but not limited to, contributing to the morbidity of the patient, negative alteration in lifestyle and negative effect on well-being. The preferred embodiments of the present invention incorporate combinations of one or more suitable means capable of providing the methods of specific cell type modulation chosen to correspond to the disease process being treated.

The preferred embodiments of the present invention incorporate any suitable means capable of providing dynamic spectral irradiance preferentially delivering therapeutic wavelengths of light with a higher probability of targeting activated cells preferentially over inactivated cells. Wherein said preferentially targeting therapeutic wavelengths include, but are not limited to, UVA1, and UVA1C. Wherein said activated cells include, but are not limited to, activated lymphocytes. Wherein said inactivated cells including, but not limited to, inactivated lymphocytes. The action spectra for a given target cell effect varies with wavelength indicating different wavelengths that have different probabilities of affecting a given target cell. Dynamic control of the wavelengths allows the preferred embodiments of the present invention to optimize the approximation of a phototherapy prescription. The different cell interaction probabilities among wavelengths allows differentiation of effect which indicates that many activated cells have altered metabolic behavior and undergo internal modifications that also change the activated lymphocytes susceptibility to light including, but not limited to, UVA1 light. In addition, certain wavelengths of light have a higher probability of targeting inactivated cells than other wavelengths. And finally, certain wavelengths of light have the same probability of targeting both activated and inactivated cells. Depending on the specificity of the light source including, but not limited to, an LED emitting a wavelength range, it is possible to target a cell type to increase the probability of preferred actions including, but not limited to, alter a specific disease, pathological process, and effect some other desired change. The preferred embodiments of the present invention incorporate any suitable means capable of providing the useful methods of applying a first wavelength in one region and then a counter effect in a second region. The useful purpose of the multiple dynamic spectral irradiance creates an effect that varies by region which is useful in systemic and organ specific diseases.

The preferred embodiments of the present invention incorporates a dynamic wavelength selection control means to provide a useful benefit including, but not limited to, dynamic sequencing wavelengths, controlling wavelengths, powering wavelengths, dynamic spectral flux, and dynamic spatial effects to provide a phototherapeutic effect.

The preferred embodiments of the present invention emit a dynamic spectral irradiance of a suitable prescribed spectral irradiance, flux, color, or any combination thereof. The emitted light is applied in a controlled manner including, but not limited to, directed application of EMR flux, to said therapeutic radiation body component targets including, but not limited to, skin, blood, endothelium, in a specific phototherapy dose including, but not limited to, 6 Joules per square centimeter, and from 3 Joules per square centimeter to 12 Joules per square centimeter, and less than about 35 Joules per square centimeter, whereby suitable methods provide a specific cellular mechanism, or mechanisms, to achieve a combination of one or more therapeutic effects, including, but not limited to, change cell populations and ratios of different types of cells, as well as to alter the at least one process occurring within the target at the time of the application of light, and subsequent processes occurring after the directed application of EMR flux.

The preferred embodiments of the present invention incorporates any suitable means including, but not limited to, mechanical translating means, mechanical rotating means, moving mirrors, multi-axis mechanical means, and external photo-irradiation means, capable of providing the useful method of targeting blood vessels and/or tissues near the surface of the skin including, but not limited to, the use of ultraviolet LEDs to deliver light through the skin into the sub-dermal capillaries. Said photo-irradiation means provides capabilities to adjust the ratio of activated lymphocytes in the blood of patients with diseases including, but not limited to, target diseases, and SLE. The preferred embodiments of the present invention are controlled to treat a combination of one or more diseases including, but not limited to, any disease where the alteration in populations of cells within the blood from an external source would benefit or beneficially alter said target disease process. The preferred embodiments of the present invention are combinations of one or more form factors, including, but not limited to, an external phototherapy form factor means, and an internal phototherapy form factor. Wherein said external phototherapy form factor means including, but not limited to, a phototherapy chamber, a hand-held phototherapy form factor with the desired light source, a flexible light emitting blanket form factor, a photophoresis form factor means. Said phototherapy chamber form factor exposes the patient inside the phototherapy chamber to the light delivery means emitting the desired spectral irradiance at a prescribed flux over a prescribed time period. Said hand-held device is used by manually directed application of phototherapeutic spectral irradiance of prescribed spectral irradiance at prescribed spectral power densities over a prescribed time period. Said phototherapeutic blanket is used by automated directed application of phototherapeutic wavelength flux of prescribed power densities and durations. Said photophoresis therapeutic means is used by preparing a patients blood in using sterile means, separating the blood components, treating one or more blood components with chemical or biological agents, irradiating one of said blood components, reconstituting the blood by mixing a plurality of said separated blood components, returning said blood to patient, measuring patient response, and analyzing results. Wherein said internal form factor means is comprised of a combination of one or more components, including, but not limited to, a phototherapeutic capsule means, a phototherapeutic catheter means, a phototherapeutic stent means, and an evacuation catheter. Wherein said phototherapeutic capsule means is made use of by introducing the phototherapeutic capsule into the gastrointestinal tract, tracking the position of said phototherapeutic capsule means, manipulating position of said phototherapeutic capsule means, activating directed application of wavelengths means responsive to a position control means, optionally deactivating directed application of phototherapeutic wavelengths means, and eliminating or otherwise recovering phototherapeutic capsule means. In an alternative embodiment of said phototherapeutic capsule means the device is active upon introduction of the capsule into the body and therefore, does not require activation step(s) or deactivation step(s). Capsule activation and capsule deactivation cycles occur in one modality of operation by sensing the variations in pH within the gastrointestinal track or other parts of the human body using detections means. Wherein said phototherapeutic catheter means is used by introducing said phototherapeutic catheter into the body cavity, positioning said phototherapeutic catheter, activating phototherapeutic wavelength generating means, optionally deactivating phototherapeutic wavelength generating means. Said preferred embodiment of the present invention incorporates any suitable means capable of providing the useful methods to ease the swallowing of the capsule by a patient.

### Disease Conditions

SLE-like photherapies incorporate elements including, but not limited to, phototherapeutic light including, but not limited to UVA1 light is delivered to a combination of one or more of said phototherapy targets using a combination of one or more light sources. Wherein said light sources are comprised of combinations of one or more combinations of optical components, light sources, and optical filters, over combinations of durations and intensities. Wherein said therapeutic light sources is comprised of combinations of one or more light sources including, but not limited to, light-emitting diode (“LED”), organic light-emitting device (“OLED”), nanocrystal, nanoparticle, carbon nanotube, laser, fluorescent bulb, and incandescent bulb. Wherein said optical filters is comprised of combinations of one or more filters including, but not limited to, low-pass filter, band-pass filter, and high-pass filters. Wherein said optical filters have optical filter characteristics comprised of filter characteristics including, but not limited to, adaptive filter characteristic, dynamic filter characteristics, and static filter characteristics. Wherein said optical components is comprised of combinations of one or more components including, but not limited to, mirror, fiber-optic, integrating spheres, integrating optics, prism, and diffraction gratings.

Said SLE-like phototherapies have capabilities including but not limited to delivering phototherapeutic light to phototherapy targets including, but not limited to, whole body, partial body, external body surface, and internal area including but not limited to external orifice, nasal passages, mouth, ear, anus, urethra, sweat gland, open wound, intentionally created opening in the body surface, internal cells, internal tissue, fluid, organs, regions of organs, and tissue.

Said SLE-like target diseases have disease characteristics including, but not limited to, responsiveness to UVA1 phototherapy treatments.

Said SLE-like target diseases are comprised of combinations of one or more diseases including, but not limited to, rheumatoid arthritis, scleroderma (a.k.a sleroderma), autism, cerebral palsy, post-traumatic stress disorder (“PTSD”), schizophrenia, bipolar disorder, depression, seasonal affective disorder (“SAD”), dysthymic disorder (“dysthymia”), nerve regeneration, nerve or neural tissue conditions, immune deficiency conditions, cancer, immune system modulation, acne, psoriasis, sexual disorders, ear diseases, eye diseases, esophageal disease, bone disease, tendon disease, muscle disease, adenoid disease, tonsil disease, hair follicle disease, plant diseases, circulatory diseases, emotional disorders, cognitive disorders, sexual disorders, reproductive disorders, digestive disease, developmental disorders, aging disorders, asthma, plant disease, and animal diseases.

Wherein said scleroderma disease is comprised of one or more scleroderma disease types including but not limited to, localized scleroderma, diffuse scleroderma, and Morphea,

Wherein said bipolar disorder disease is comprised of one or more bipolar disorder disease types including but not limited to, Type I bipolar disorder, and Type II bipolar disorder.

Wherein said depression disease is comprised of one or more depression disease types including but not limited to, acute depression, and chronic depression.

Wherein said nerve or neural tissue conditions is comprised of one or more nerve or neural tissue condition types including but not limited to, post-contusion (“bruising”) injury, hemi-section transection, full nerve transection, post-nerve repair, post-nerve graft, peripheral nerves, central nerves, spinal cord, dorsal root ganglion, a collection of nerve tissues, a combinations or groupings of nerve cells, axons, and nerve tissue. Wherein said post nerve repair type of said nerve or neural tissue condition is associated with repair types including, but not limited to, nerve or neural tissue re-approximation using re-approximation surgical devices. Wherein said re-approximation surgical devices are comprised of on or more surgical device including, but not limited to, sutures.

Wherein said immune deficiency conditions disease is comprised of one or more immune deficiency conditions disease types including but not limited to, hereditary, and acquired. Wherein said hereditary type of immune deficiency condition is a combination of one or more hereditary immune deficiency diseases including, but not limited to, chronic granulomatous disease. Wherein said acquired type of immune deficiency condition s a combination of one or more hereditary immune deficiency diseases including, but not limited to, Acquired Immune Deficiency Syndrome (“AIDS”), HIV, any condition where immune deficiency is induced by external immune deficiency agent. Wherein said external immune deficiency agent is a combination of one or more agents including, but not limited to, medication administration, cancer, or disease.

Wherein said cancer disease is comprised of one or more cancer disease types including but not limited to, cutaneous T-cell lymphoma, primary lymphoma, and solid tumors.

Wherein said acne disease is comprised of one or more acne disease types including but not limited to, nodular, and cystic.

Wherein said tissue disease is comprised of combinations of one or more diseases including, but not limited to, finger nail disease, toe nail disease, cartilage disease, glandular disease, wart disease, viral disease, parasite disease, bacterial disease, pestilent disease,

Said SLE-like target phototherapies are comprised of one or more phototherapies including, but not limited to, plastic surgery phototherapies, cellular organelle phototherapy, cell phototherapy, tissue phototherapies, organ phototherapy, immune system modulation phototherapy, cloning enhancement, artificial insemination, normal and/or atrophied muscle stimulation, blood flow phototherapy, sexual enhancement phototherapy, brain region phototherapy.

SLE-like Phototherapies is comprised on combinations of one or more phototherapies devices and methods useful in performing effects including, but not limited to:

Cancer phototherapies

Primary treatment of any cancer type shown to benefit from UVA1 light application including but not limited to, Cutaneous T-Cell Lymphoma, Primary Lymphoma, or solid tumors,

Immune System treatment by whole-body UVA1 light administration

Post-chemotherapy skin and tissue rejuvenation (including but not limited to whole-body and local application—internal and external)

Post-radiation skin and tissue rejuvenation (including but not limited to whole-body and local application—internal and external)

Brain Region Photoirradiation using one or any combination catheters or fiber-optic conduits with one or more combinations of light sources

Tissue or Organ Photoirradiation using one or any combination catheters or fiber-optic conduits with one or more combinations of light sources

Plastic Surgery Applications

Including but not limited to:

Flaps—free, rotational or any other kind described or not

Graft tissue

Graft tissue preparation and maintenance

Wound healing—including but not limited to chronic or acute wounds such as diabetic foot ulcers, decubitus[SP?] ulcers, or sacral ulcers

Immune System Modulation

For normal patients

As a prophylactic treatment against conditions including but not limited to cancer, infection, aging, or fatigue

Description of Individual Phototherapies

### Rheumatoid Arthritis

Rheumatoid arthritis is treated in a similar manner as described for Lupus UVA1 phototherapy and other said SLE-like diseases benefit from the same effects of the UVA1 light mechanisms. The rheumatoid arthritis phototherapy includes combinations of one or more phototherapy methods including, but not limited to, any of said therapeutic light sources or combinations of delivery methods to provide whole-body phototherapy, localized phototherapy to one or more affected locations including but not limited to a hand, a finger, a knee, or multiple affected joints

To effectively deliver the therapy to affected joints, the lights sources are incorporated into combination of phototherapy device form factors including, but not limited to, flexible, fabric, wrap, and strap.

### Scleroderma

Scleroderma phototherapy encompasses a variety of Scleroderma conditions and Scleroderma manifestations. Wherein said Scleroderma conditions are combinations of one or more conditions including, but not limited to, localized Scleroderma, diffuse Scleroderma, morphea, systemic sclerosis.

Scleroderma can be categorized into various forms pending on location, symptoms and severity. Localized Scleroderma or morphea is characterized by epithelial plaques and vascular atrophy confined to a specific region or limb. Currently there isn't a viable treatment available, however, it has been determined by many published studies that UVA-1 phototherapy serves as an effective therapy.

Although the etiology of systemic sclerosis onset is unknown there are certain fluctuations of bio-mediators that indicate affliction. Polymorphism of regulatory genes COL1A2 and TGF-β1 for example are commonly observed in limited scleroderma patients. (Jimenez and Derk, 2004) This is potentially a trigger the increased synthesis of collagen I and III activity in fibroblasts.

Regarding scleroderma immunology, there is evidence that CD34 dendritic cells in the dermis are significantly reduced in the presence of sclerotic legions. (Camacho et al, 2001) The use of UVA1 may stimulate immunomodulation inducing keratinocytes, epidermal Langerhans cells, mast cells, and skin-infiltrating T cells. (Stege et al, 1997, Grewe et al, 1995, Morita et al, In Press)

To elaborate, UVA1, affects biological processes including, but not limited to repair collagen metabolism, vascular activity, autoimmune. Wherein said autoimmune alteration is affected by UVA1 biological mechanisms including, but not limited to, alteration by depleting skin-infiltrating T cells and proinflammatory cytokines; IL-6, IL-1, as well as inducing a shift of the balance between proto-oncogenes and tumor suppressor genes towards cell death and endothelial transformation. (Kreuter et al, 2006, Breuckmann F, et all 2004, Wlaschek et al, 1994, Gruss et al, 1997).

Said scleroderma phototherapy is delivered both whole body and in a variety of form factors to deliver light to one or any combination of regions on or within the body. Specifically, some of the more serious manifestations of Scleroderma are related to the contractures that form in a variety of places throughout the body. More commonly manifestations are found on the hands, wrist and along the forearm, depending on the severity and extent of the disease, both in localized and diffuse Scleroderma. UVA1 light has been shown to provide multiple benefits for people afflicted with Scleroderma.

The hands are usually the first location to present in new-onset Scleroderma and is the most debilitating morbidity described by patients. Thus far, prior research indicates efficacy using UVA1 phototherapy at a high dosage (130 J/cm2), medium dosage (40-70 J/cm2) and low dosage (10-30 J/cm2) therefore suggesting no discerning relationship between intensity and therapeutic potential. The UVA1 therapy is effective and safe, and this section describes a LED-containing device that is anatomically designed to deliver phototherapy to the hands of Scleroderma patients to significantly reduce the inflammation and pain due to excessive fibrosis and improve the quality of life of affected patients.

The therapy is commonly described utilizing the energy delivered to the affected region. This is done using Joules/cm² as a standard of measurement. [A BETTER DESCRIPTION WILL BE OUR PHOTOTHERAPY PRESCRIPTION] The commonly published dosage increments for UVA1 phototherapy delivery to the hand are subdivided into high dosage (120-150 J/cm2), medium dosage (40-70 J/cm2) and low dosage (10-30 J/cm2). The general experimental protocol calls for light administration three to five times weekly for between eight to ten weeks. The treatment session averages 30 minutes each.

Scleroderma Hand Phototherapy Device Description:

The phototherapy chamber described here emits photometrically calibrated light within the UVA1 range (340 nm-400 nm). This device is a reliable light source calibrated to deliver a prescribed quantity of light in a prescribed period of time. It is designed to be ergonomic so as to minimize the risk of pressure wound development due to the decreased vascularity and increased sensitivity of the hands of Scleroderma patients to breakdown. This will be delivered through a combination of inflatable and/or expandable hand positioning devices within the chamber that allows for light to be delivered through the hand mount in addition to the light within the chamber. However, as the contractures in the hands resolve over time as a result of the UVA1 phototherapy, the hand mount is able to expand as the hand and digits gain mobility allowing for some mild physical therapy to occur while the phototherapy is going on over time. The stretching and expansion of the hand mount helps to restore movement to the hand as the scarring contractures decline.

This hand phototherapy chamber is specifically designed for ease of use and increased reliability for treatments involving the hands and forearms. The device has the capability to maintain a single setting for the duration of the study, and the capability to be reprogrammed by factory trained technicians for subsequent studies. The light delivery can be achieved as mentioned above using one or multiple combinations of LEDs, OLEDs, lasers, incandescent sources or fiberoptic conduits in multiple form factors including but not limited to a chamber for the hands to reside in, gloves made of any flexible, rigid or semi-rigid material.

### Autism

Autism is postulated to occur following a semi-normal development in children at which point some unknown stimulus causes normal brain development to slow or halt. The use of UVA1 phototherapy in any combination of delivery methods as described above allows the delivery of combinations of one or more therapeutic light in the UV or visible light range to one or multiple parts of the brain gaining access through any number of means including but not limited to threading of catheters or fiberoptic conduits through arteries or veins, or by direct irradiation to the surface of the brain following the creation of a burr hole in the cranium or through irradiation after access the ventricular system of the brain by descent through the central sulcus. The placement of therapeutic light source in the cranium. Said therapeutic light source in the cranium require a power source, including but not limited to magnetic coupling, wire ports, batteries, chemical conversions using materials from the body.

Ideally, this treatment is initiated at the first sign of the disease presenting or following a discovery of the cause of the disease at which point in time a test were devised to test for the diagnosis before the patient suffered development delay and mental retardation. Autism phototherapy is provides beneficial light effects to brain tissue or any other tissue shown to be involved in the pathophysiology of the disease.

One such example is when the cause is related to an interruption in blood supply, oxygen supply or some other critical pathway in the brain resulting in the death or impaired survival of brain tissue including but no limited to neurons, dendritic cells, or glia. At that point in the autism disease process, the autism phototherapy device is inserted into the body by any of the above described means and threaded through the vasculature by interventional radiology technique to the site or sites that are affected so that light, including but not limited to UVA1 light, is delivered for a period of time deemed to recover the tissue or cells and restore function or minimize long-term damage. As has been shown in studies referenced herein, UVA1 light has been shown to possess some neuronal cell survival capabilities, decreasing the death of cells in culture. In addition, it may possess the ability to promote cell and axonal growth, which improves the outcome of this disease if therapy were initiated with UVA1 light to affected parts of the brain and/or a whole body external phototherapy method.

### Cerebral Palsy

Cerebral Palsy benefits from UVA1 phototherapy in a similar way as Autism. Cerebral Palsy is treated a phototherapy device, including but not limited to UVA1 phototherapy.

Each of these disorders [AUTISM AND CEREBRAL PALSY] can be commonly grouped as mental illnesses and collectively involve disruptions in neurotransmitters within the brain, genetic abnormalities and changes in the surrounding external environment, including but not limited to decreased sunlight resulting in SAD. Each condition has unique and/or similar alternations in neurotransmitters however, research has shown that the application of UV light has the ability to effect these concentrations in each condition in a manner that improves or alleviates the condition and/or its symptoms.

The use of phototherapy chamber that contained any combination of emitting sources to deliver light as a constant output phototherapy for a given period of time or with patterns and sequences of light can be employed to help treat the above list mental illnesses. In addition, these emitting sources includes but are not limited to the building of a room or chamber incorporating an emitting source such as LEDs throughout the chamber in a manner able to provide constant output of a beneficial kind of therapeutic light and/or in sequences and/or patterns known to help treat the given condition. For example, lights in sequences and patterns have been shown to positively treat PTSD and UV light can be used to help treat SAD.

The use of UV light is employed to not only treat the diseases underlying problems but also modulate the mood of afflicted patients in positive directions and/or stabilize moods in patients afflicted with conditions such as bipolar disorder or dysthymia where the mood can shift in multiple directions.

### Nerve Regeneration

Nerve Regeneration phototherapy is useful in therapies to treat nerve damage and injury conditions. Wherein said nerve damage and injury conditions is comprised of combinations of conditions including, but not limited to, post-contusion (bruising) injury, hemi-section or full nerve transaction, post-nerve repair (re-approximation with sutures or any other means), post-nerve graft, peripheral and central nerves of any variety, spinal cord, dorsal root ganglion, any collection of nerve tissue, any combination or grouping of one or more nerve cells, axons or nerve tissue.

### Plastic Surgery Phototherapy Application

Plastic surgery phototherapy applications are combinations of one or more SLE-like phototherapies procedures performed with plastic surgery techniques including, but not limited to, flaps, free flaps, rotational flaps, grafts, graft preparation, wound healing, chronic wound healing, acute wound healing, diabetic foot ulcers, decubitus ulcers, or sacral ulcers

### Nerve or Neural Tissue Therapy

This use of UVA1 light includes but is not limited to the following conditions associated with nerve damage or injury:

Post-contusion (bruising) injury

Hemi-section or full nerve transection

Post-nerve repair (re-approximation with sutures or any other means)

Post-nerve graft

Includes but is not limited to peripheral and central nerves of any variety

Includes but is not limited to the spinal cord, dorsal root ganglion, or any collection of nerve tissue or combination or grouping of one or more nerve cells, axons or nerve tissue

Some of the uses included within this section have been derived from data generated utilizing UVA1 LEDs of various wavelengths on neural cell cultures.

The use of LED's would take the same forms as mentioned above including but not limited to any single or combination device using LEDs, OLEDs, laser, fluorescent or incandescent sources as well as catheters, fiber optic conduits, or otherwise.

As listed above, the UVA1 light would be applied directly to an injured nerve, nerve cell body, axon or ganglion or area of neural tissue, including but not limited to areas of the brain, so as to gain the beneficial effects of UVA1 light including but not limited to the neuroprotective effects seen in experiments as reported in this provisional, the generation of ATP to provide energy to the cell for repair or growth purposes, and all anti-inflammatory, healing benefits known to be derived from the application of UVA1. In addition, this could be applied to grafted nerves, nerves where the axon had been reapproximated with suture or any other means, nerves damaged by impingement, including but not limited to a bulging or herniated disk in the vertebral system, carpal tunnel in the wrist, or nerves damaged by direct impact and primary and/or secondary contusion injury, including but not limited to spinal cord compression, vertebral fractures, or otherwise.

In addition, per the data listed in this provision, the application of UVA1 using a variety of form factors would allow for the benefit of UVA1 that is axon polarization and axon growth of an undifferentiated neural cell including but not limited to stem cells, nerve stem cells, hippocampal neurons, central or peripheral nerves or other wise. Also, UVA1 would be applied to boost overall cell survival to an area of infarcted or damaged nerve or brain tissue area and/or work to provide nerve regeneration capabilities.

### Plastic Surgery Phototherapy Devices and Methods

This covers the use of UVA1 LEDs, lasers, fiberoptic conduits, OLEDs or any other combination to provide the following:

Prepare flaps/grafts for transfer by:

Increasing reactive oxidant species (ROS) scavenging

Increase ATP in cells to provide energy and increased survival capabilities after the transfer

Increased anti-inflammatory response to improve the flap/grafts ability to handle the stress of surgery, transplantation and healing and any resultant infection or other event that may occur postoperatively

Dilation of blood vessels, including but not limited to arteries, veins, arterioles, capillaries, and/or choke vessels, secondary to heme oxygenase (HO) induction and any other vasodilatory capabilities of UVA1 light

Maintain a flap/graft after elevation or during transfer by the same mechanisms as in item number 1 above, but also by providing singlet oxygen to drive aerobic metabolism so that no shift to anaerobic metabolism occurs, as typically does in these situations. This results in a decrease of lactic acid (lactate) that usually occurs after flap/graft is elevated in preparation for a transfer or after a transfer.

Increase graft/flap survival after transfer to the donor site by same mechanisms as in number one or two.

Increase recovery and healing of the donor site and flap/graft for the same reasons as in number one or two above.

Increase the immune protection in the situations listed in numbers one through four and decrease infection risk and/or occurrence by boosting immunity in the area where UVA1 is applied by increasing macrophage function, boosting cell-mediated immunity and all other means by which UVA1 has been shown to improve immune system function.

Improve the healing capabilities and allow for the granulation of tissue within acute and or chronic wounds by the mechanisms described in numbers one through five and by the following means including but not limited to directly enhancing blood flow to a would, increase immune system function in the area of the wound, increase nerve regeneration, survival and in-growth in the area of a wound, and stimulating the granulation and epithelialization of an exposed wound due to the body's natural protective response to UV light exposure.

Decreased incident of scar formation and/or decreased overall amount of scar formed following operations or for the use in the treatment of existing scars including but not limited to patients who are prone to the formation of hypertrophic and/or keloid scars or any other undesired scar. This would be due to the following mechanisms including but not limited to the increased induction of the enzyme Collagenase I, as seen in the Scleroderma UVA1 phototherapy, and the anti-inflammatory effects of UVA1 which would help to decrease the formation of scar and the inflammatory reaction that occurs after operations, suturing or skin injury for any reason.

### Methods for Plastic Surgery Applications:

The following includes but is not limited to the following form factors by which the above plastic surgery applications can be derived:

By application of a mesh device with multiple LEDs, lasers, OLEDs, fiberoptic conduits, etc applied to an area of flap/graft, internal inflammation in area of the flap before elevation via means including but not limited to a percutaneous route, wrapping of the flap/graft after elevation and/or during the delayed flap/graft procedure.

Also, placement of an LED, laser, OLED, fiberoptic system, etc over a cutaneous flap/graft at donor site and/or inside tissue or flap/graft after transfer to donor site for several days to increase flap/graft survival as described in the above Plastic Surgery section in numbers one through five.

When treating acute or chronic wounds, any UVA1-based light system as described above would be taped or placed within or over a wound site and applied either constantly until desired response achieved or for one or multiple therapeutic sessions throughout the day. The application of light therapy could precede, follow or be done during the debridement of a wound, which is a common treatment used to work to treat and heal a chronic would through the removal of dead or dying or infected tissue as well as bacteria. In addition, following the debridement of chronic wounds, skin grafts may often be used to cover the site, either split-thickness or full thickness grafts. The same application of UVA1 to the newly grafted tissue would be used to enhance the “take” or healing of grafted tissue to the chronic wound site and to allow for enhance granulation of the tissue due to the means mentioned above.

Following debridement of a wound, the grafting of a wound or incision or any other means of treating an acute or chronic wound, frequently a “wound vac” is applied, which is a device that applies suction to an area after sponge-like foam as been placed within the wound and sealed in using a specialized form of tape. A special foam connector with suction attached is then placed over the sealed area following the placement of a small hole in the taped-seal. The connector is then also sealed over and when the vac is turned on applies direct suction and a seal to the area to clear any fluids within the wound and improve healing.

This method also describes the use of LEDs, OLEDs, lasers or any fiberoptic means or combinations of the following within the foam sponges placement within the wound, in the connector or placed in a mesh, gauze or any other means in the base of the wound to allow for the direct application of UVA1 light to the wound while the wound vac is in place and the suction is applied.

This would allow for all of the beneficial means of UVA1 light to a given region in addition to the added benefit of the clearance of fluid and debris from the area from the vac creating an additional benefit to the combination of both applications.

As in numbers one through five, the UVA1 light application using LED's, lasers, OLEDs or any fiberoptic conduit can be used to increase survival and regeneration of nerve tissue present or grafted to a given area to increase sensation and motor function as well as the sympathetic regulation of blood flow and blood vessels that is typically lost following an injury or the elevation and transfer of a flap/graft to a given area. This would allow for improved blood flow to a flap/graft, which has been shown to increase the take and survival of the transferred flap/graft.

### Immune Deficiency Conditions

The use of whole-body or localized UVA1 therapy could be used to improve or treat hereditary immune deficiency conditions including but not limited to Chronic Granulomatous Disease. This would work in the previous condition as Chronic Granulomatous Disease results from a deficiency of enzymes that produce the reactive oxidant species within phagocytic and other cells of the immune system used to kill not only infectious organisms but also breakdown and destroy debris. This includes but is not limited to a deficiency of singlet oxygen. UVA1 has been shown to enhance the abilities of the immune system to fight off infection and has also been shown to generate singlet oxygen within cells, which would help to correct the defect in Chronic Granulomatous Disease.

The use of UVA1 light could also be used in acquired immune deficiency conditions including but not limited to Acquired Immune Deficiency Syndrome (“AIDS”), HIV or any condition where immune deficiency is induced by something including but not limited to medication administration, cancer, or any other disease. UVA1 would function as an immune boosting and/or anti-inflammatory means to help patients with these conditions fight off infection, increase survival and also secondarily increase the efficacy of treatments for the primary condition or other comorbidities within the patient.

### Cancer

Primary treatment of any cancer type shown to benefit from UVA1 light application including but not limited to Cutaneous T-Cell Lymphoma, Primary Lymphoma, or solid tumors

Immune system treatment by whole-body UVA1 light administration

Post-chemotherapy skin and tissue rejuvenation (including but not limited to whole-body and local application—internal and external)

Post-radiation skin and tissue rejuvenation (including but not limited to whole-body and local application—internal and external)

UVA1 when used in any of the previously described delivery systems would be used to improve the state of the immune system during treatment for cancer as well as acting as a primary treatment for the cancer when applied in a whole-body or localized means. Some cancers have been shown to result following a deficiency in the immune system, whether a primary or secondary cancer. UVA1 light phototherapy would also be a critical adjuvant therapy during and following and before chemotherapy and radiation as these have a devastating effect on the immune system, which could have an indirect effect on the ability of the body to fight off cancer.

Following the induction of chemotherapy, the immune system is severely damaged, as it is included in the list of fast growing cells which chemotherapy is targeting in addition to the cancer. However, it's been shown that the immune system has a major role in not only preventing and treating cancer, but also being able to be alerted to the presence of a cancer and eliminating it following a means such as tumor lysis or disturbance by mechanical or pharmacologic means. UVA1 light could be used in a whole-body or localized fashion to boost the immune systems ability to fight the cancer once the cancer is recognized either by a smaller dose of chemotherapy or some other means of mechanical or chemical lysis, as opposed to treating with full doses of chemotherapy and nearly completely eliminating the body's ability to fight off infection. Should full chemotherapy induction be used and the immune system decreased, daily or constant whole-body or other schedule of UVA1 phototherapy would be used to enhance the remaining immune systems ability to recognize and fight infection while also providing ATP and energy as well as anti-inflammatory benefits to the patient to aid in recovery from chemotherapy and/or radiation as well as fight off infection and the cancer itself.

Post-chemotherapy skin, tissue, and body rejuvenation—this would use the beneficial effects of UVA1 light when applied whole-body, direct or indirect internal or external irradiation to rejuvinate the skin and/or body following the use of chemotherapy as well as the injury that can occur at the site of chemotherapy injection via an intravenous catheter or needle injection, as chemotherapy is caustic to the skin and tissues.

Post-radiation skin, tissue, and body rejuvenation—as above, the use of UVA1 light therapy either whole-body, localized external or localized internal applications to areas of radiation therapy would be used to help the body recover from the devastating secondary effects of radiation to normal as well as targeted skin and tissue. This is a terrible skin reaction that is very similar to sunburn and would benefit from the anti-fibrotic mechanisms of UVA1 such as the induction of Collagenase I as seen in the Scleroderma therapy to prevent skin and tissue contracture following radiation therapy, also it can result in a decrease in inflammation to a given area.

In breast reconstruction, for example but not limited to this example, or any plastic surgery reconstruction procedure following local tissue excision and/or post-surgical chemotherapy and/or radiation

UVA1 light would increase the healing of these areas both at the local treatment and/or reconstruction site as well as within the body if the UVA1 were applied to the target area or areas that were affected in a “bystander” means following chemotherapy or radiation.

UVA1 would be applied after the chemotherapy and/or radiation to allow for an increased recovery time and/or increased time at which reconstruction of an area could occur as these procedures are typically delayed due to the long-lasting negative effects of chemotherapy and radiation on the body's ability to heal following an operation.

UVA1 would also result in faster healing times and decreased scar formation following reconstruction or operation of an area or patient that underwent chemotherapy or radiation. This includes flaps/grafts that were radiated or were present during chemotherapy and could benefit from the effects of UVA1 to increase their survival and decrease the likelihood of a repeat operation or new flap/graft.

### Immune System Modulation

In normal patients, or patients with any other condition or conditions, this method includes but is not limited to the use of UVA1 light whole-body and/or localized photoirradiation to increase immune system surveillance and/or suppression of cancer development. This is based on published literature showing an increased incidence of cancer in patients that are immunosuppressed either primarily or secondarily, inherited or acquired or any combination thereof. In addition, the use of UVA1 would be able to decrease the effects of aging, increase lifespan and also result in a decreased incidence of infection in patients given the already known effects of UVA1 light. It would also be able to boost energy in patients due to the increased production of ATP that occurs.

### Acne

This including but is not limited to the use of localized UVA1 application to affected parts of the body that are afflicted with nodular, cystic or any other form of acne. This would help the body to fight off the known infectious agent present, propionibacterium acnes or any other infectious agent present, to help the body heal the area. It would also decrease the inflammation and secondary scarring that occurs from the presence of comedones, cystic acne or other lesions due to the anti-inflammatory natures of UVA1 light as well as the increased induction of Collagenase I.

### Psoriasis

This employs the penetrating effects of UVA1 light to penetrate beneath the psoriatic lesions and to modulate the immune reaction that is occurring, downregulate inflammation and by any other mechanisms of UVA1 to help resolve the lesions. Also, the anti-inflammatory nature of UVA1 light would help to deal with the effects of psoriatic arthritis that can occur and would be delivered by whole-body and/or localized means.

### Brain Region Phototherapy

This would use catheters with any combination of LEDs, OLEDs, lasers or other means on tips either individually or in arrays and also including but not limited to the use of LEDs, lasers or OLEDs to deliver UVA1 or any other form of light via fiberoptic conduits either within the vasculature, by direct percutaneous or intraoperative penetration or any other means of accessing the body or areas of the body, skin or tissue or hollow cavities or fluid filled areas or cavities.

The use of various sized catheters and/or fiberoptic conduits to deliver light in one or multiple branches of blood supply to areas of the brain by following the larger blood vessels (arteries or veins or any other type of blood vessel) to get to smaller vessels. This would allow a combination of various sized catheters and/or fiberoptic conduits to be used to create and envelopment of UVA1 light around a given brain region and/or the watershed area of the blood vessels being used to access the area. Arteries or veins could be used for access. Also, access to the ventricular system of the brain could be used by entry through the skull into the central sulcus or through the skull to apply the aforementioned therapy to areas of the subdural or epidural locations and the blood supplies within those regions.

Device would allow the placement of any combination of catheters with LED tips or arrays, OLEDs, lasers or fiberoptic conduits supplied by LEDs, OLEDs, lasers, fluorescent or incandescent sources or any combination of these with or without filters. The catheters would have light devices placed not only at the tip but also mounted within the catheter body so that light could be emitted from along the length of the catheter, not just at the tip.

The uses include but are not limited to the use of UVA1 light to increase ATP to an infarcted area or area where an insult of some kind occurred, including but not limited to a stroke, a compression due to an overlying or local hematoma or tumor causing compression injuries that choke or restrict blood supply, etc. In addition, the anti-inflammatory elements, immune modulating elements as well as neuroprotective and neuroregenerating elements of UVA1 would also be able to be applied to the area. This would be used in conditions including but not limited to stroke, autism, cerebral palsy, neurodegenerative conditions, multiple sclerosis, etc.

### Tissue or Organ Phototherapy

The above mentioned catheter, fiberoptic conduit based device would be sued for any tissue or organ to provide light therapy to tissue or organs via their enveloping or supplying/draining blood supply (including but not limited to arteries, veins, vascular bundles, capillary networks, etc.)

Conditions that would benefit from this therapy include but aren't limited to a myocardial infarction, a pulmonary embolism, or any other infarction caused by an emboli.

### Nanocrystal or Embedded Light Structure

The light source could be nanocrystal that's activated in a variety of means including but not limited to external vibration, piezoelectric means, magnetic filed, radiofrequency, and/or can be one or multiple combinations of nanocrystals with or without LEDs, OLEDs, micro-LEDs, LCDs (liquid crystals), lasers or any of the previous and/or incandescent sources sent into suture by fiberoptic conduit.

The suture filament would be either the “fast” type suture with reverse barbs allowing the suture to be pulled in one direction before lodging in tissue or normal suture material.

The light source could connect to an exposed end of the suture using a battery or other power source to power the light devices within the embedded suture. The suture could be powered by a microcomputer or some other wired or nonwired means that allowed for constant UVA1 light application or sequencing and patterning of light within the wound. A fiberoptic means would allow for multiple open areas along the wire where UVA1 light could be emitted within the wound where the suture was placed. For any of the above mentioned form factors, the end or ends of the suture could be left exposed on the surface of the skin and would have a connector in place to allow for the hooking of a power or light source to that area.

The benefit of having UVA1 light emitted from the suture includes but is not limited to the anti-inflammatory properties of UVA1 light provided within the healing wound, the anti-scar elements of UVA1 light such as Collagenase I provided direct to the incision, as well as the immune modulating and energy producing elements allowing for increased healing capabilities, decreased scar formation and decreased likelihood of infection.

The disease known as Scleroderma is also known as Sleroderma. Scleroderma has at least two common spellings that are used interchangeably in published journal articles, granted patents, and patents applications.

### REFERENCES

Kochevar I. E. Primary processes in photobiology and photosensitization. In Krutmann J and Elmets CA Eds. Photoimmunology Blackwell Science: Cambridge, 1995, pp 19-33.

Bruls W A G, Leaper H, Van Der Leun J C, Berrens L. Transmission of human epidermis and stratum corneum as a function of thickness I the ultraviolet and visible wavelengths. Photochem Photobiol 1984; 40: 485-494.

Morito A, Grewe M, Grether-Beck S, Olaizola-Horn S, Krutmann J. Induction of proinflammatory cytokines in human epidermoid carcinoma cells by in vitro ultraviolet A1 irradiation. Photochem Photobiol 1997; 65: 630-635

Chen D, Toone W M, Mata J, Lyne R, Burns G, Kivinen K, Brazma A, Jones N, Miler J. Global transcriptional responses of fission yeast to environmental stress. Molecular Biology of the Cell 2003;14:214-229.

Jamieson D J. Saccharomyces cerevisine has distinct adaptive responses to both hydrogen peroxide and menadione. J Bacteriol 1992;174:6678-6681.

Gasch A P, Spellman P T, Kan C M, Carmel-Harel O, Eisen M B, Storz G, Botstein D, Brown P O. Genomic expression programs in the response of yeast cells to environmental changes. Mol Biol Cell 2000;11:4241-4257.

Causton H C, Ren B, Koh S S, Harbison C T, Kanin E, Jennings E G, Lee T I, True H C, Lander E S, Young R A. Remodeling of yeast genome expression in response to environmental changes. Mol Biol Cell 2001;12:323-337.

Cam H P, Noma K, Ebina H, Levin H L, Grewal S I S. Host genome surveillance for retrotransposons by transposon-derived proteins. Nature 2008;451:431-436.

Tyrrell R M. Solar ultraviolet radiation: an oxidizing skin carcinogen that activates heme oxygenase-1. Antioxidants & Redox Signaling 2004; 6: 835-840.

Lindgard A, Lundberg J, Rakotonirainy O, Elander A, Soussi B. Preservation of rat skeletal muscle energy metabolism by illumination. Life Sciences 2003; 72: 2649-2658.

Klotz L O. Oxidant-induced signaling: effects of peroxynitrite and singlet oxygen. Biol Chem 2002; 382: 443-456.

Briviba K, Klotz L O, Sies H. Toxic and signaling effects of photochemically or chemically generated singlet oxygen in biological systems. Biol Chem 1997; 378: 1259-65

de la Fuente, Richaud-Patin Y, Jakez-Ocampo J, Gonzalez-Amaro R, Llorente L. Innate immune mechanisms in the pathogenesis of systemic lupus erythematosus (SLE). Immunology Letters 2001; 77: 175-180.

McGrath H Jr., Bak E, Michalski J P: Ultraviolet light prolongs survival and Improves immune function in New Zealand Black/New Zealand White F1 Hybrid mice. Arthritis Rheum 1987; 30(5): 557-561.

McGrath H Jr, Bak E, Zimny M L and Michalski J P. Fluorescent light decreases autoimmunity and improves immunity in B/W mice. J Clin Lab.Immunol 1990; 32; 113-116.

McGrath H Jr.: Ultraviolet-A1 irradiation decreases clinical disease activity and autoantibodies in patients with systemic lupus erythematosus. Clin Exp Rheum 1994; 12: 129-135.

McGrath H Jr., Bell J M, Haynes M R, Martinez-Osuna P: Ultraviolet-A (320-400 nm) irradiation in systemic lupus erythematosus. Curr Ther Res 1994; 55: 373-381.

McGrath H. Jr., Martinez-Osuna P, Lee F A: Ultraviolet-A1 (340-400 nm) irradiation Therapy in systemic lupus erythematosus. Lupus 1996; 5: 269-274.

Polderman M C A, Huizinga T W J, Cessie S L E, Paval S. UVA-1 cold light treatment of SLE: a double blind, placebo controlled crossover trial. Ann Rheum Dis 2001; 60: 112-115.

Polderman M C A, le Cessie S, Huizinga T W J, Pavel S. Efficacy of UVA-1 cold light as an adjuvant therapy for systemic lupus erythematosus. Rheumatology 2004; 43: 1402-1404.

Szegedi A, Simics E, Aleksza M, Horkay I, Gaál K, Sipka S, Hunyadi J, Kiss E. Ultraviolet-A1 phototherapy modulates Th₁/Th₂ and Tc₁/Tc₂ balance in patients with systemic lupus erythematosus. Rheumatology 2005; 44: 925-931.

Molina J F, McGrath Jr H. Long-term UV-A1 irradiation in SLE. J Rheumatol 1997; 24: 1072-1074.

McGrath H Jr.: Elimination of anticardiolipin antibodies and cessation of cognitive decline in a UV-A1 irradiated systemic lupus erythematosus patient. Lupus 2005; 14: 850-861.

Menon Y, McCarthy K, McGrath H Jr. Reversal of brain dysfunction with UV-A1 irradiation in a patient with systemic lupus. Lupus 2003; 12: 479-482.

Polderman M C A, van Kooten C, Smit N P M, Kamering S W A, Pavel S. Ultraviolet-A (UVA-1) radiation suppresses immunoglobulin production of activated B lymphocytes in vitro. Clin Exp Immunol 2006; 145: 528-536.

Godar D E and Beer J Z. UV-A1-induced anuclear damage in mammalian cells. In Biological responses to ultraviolet A radiation. Valdenmar Publishing Company: Overland Park, Kansas, 1992, pp 65-75.

Beattie P E, Dawe R S, Traynor N J, Woods J A, Ferguson J, Ibbotson S H. Can St John's wort (hypericin) ingestion enhance the erythemal response during high-dose ultraviolet A1 therapy? Br J Dermatol 2005; 153(6): 1187-1191.

Liang M H, Socher S A, Larson M G, Schur P H. Reliability and validity of six systems for the clinical assessment of disease activity in systemic lupus erythematosus. Arthritis Rheum 1989; 32: 1107-1118.

Bombardier C, Galdman D, Urowitz M, Caron D, Chang C. Derivation of the SLEDAI. A disease activity index for lupus patients. Arthritis Rheum 1992; 35: 630-640

Suh, K.-S., Roh, H.-J., Choi, S.-Y., et al. A long-term evaluation of erythema and pigmentation induced by ultraviolet radiations of different wavelengths. Skin Research and Technology. 2007; 13: 360-368.

Ravnbak, M. H., Wulf, H. C. Pigmentation after single and multiple UV-exposures depending on UV-spectrum. 2007: Arch Dermatol Res. 299: 25-32

Bech-Thomsen, Niels, Poulsen, Thomas, Christensen, Find G., Lundgren, Karsten and Christian Wulf, Hans. Near-Visible-UV radiation delays UVB tumorigenesis. J. Photochem. Photobiol. B: Biol., 1994; 22: 199-123

De Fabo, Edward C., Noonan, Frances P, Fears, Thomas, and Merlino, Glenn. Ultraviolet B but not Ultraviolet A Radiation Initiates Melanoma. Cancer Research 2004; 64: 6372-6376.

Ogawa, Fumihide, Sander, Christina S., Hansel, Alfred et al. The Repair Enzyme Peptide Methionine-S-Sulfoxide Reductase Is Expressed in Human Epidermis and Upregulated by UVA Radiation. Journal of Investigative Dermatology 2006; 126: 1128-1134.

de Rie M A, Enomoto D N H, de Vries H J C, Bos J D. Evaluation of medium-dose UVA1 phototherapy in localized scleroderma with the cutometer and fast fourier transform method. Dermatology 2003;207:298-301

Camacho N R, Sanchez J E, Martin R F, Gonzazles J R, Sanchez J L. Medium-dose UVA1 phototherapy in localized scleroderma and its effects in CD34-positive dendritic cells. J Am Acad Dermatol 2001;45:697-9

Kreuter A, Hyun J, Stuecker M, Sommer A, Altmeyer P, Gambichler T. A randomized controlled study low-dose UVA1, medium-dose UVA1, and narrowband UVB phototherapy in the treatment of localized scleroderma. J Am Acad Dermatol 2006;54:440-447

Kreuter A, Hyun J, Skrygan M, Sommer A, Bastian A, Altmeyer P, Gambichler T. Ultraviolet A1-induced downregulation of human beta-defensins and interleukin-6 and interleukin-8 correlates with clinical improvement in localized scleroderma. Br J Dermatol 2006:September;155(3):600-7

Dawe R S. Ultraviolet A1 phototherapy. Br J Dermatol 2003;148:626-637

Kerscher M, Volkenandt M, Gruss C, et al. Low-dose UVA1 phototherapy for treatment of localized scleroderma. J Am Acad Dermatol 1998;38:21-26

Rombold S, Lobisch K, Katzer K, Grazziotin T C, Ring J, Eberlein B. Efficacy of UVA1 phototherapy in 230 patients with various skin diseases. Photodermatol Photoimmunol Photomed 2008;24;19-23

Stege H, Berneburg M, Humke S, Klammer M, Grewe M, Grether-Beck S, et al. High-dose UVA1 radiation therapy for localized scleroderma. J Am Acad Dermatol 1997;36:938-44

Scharffetter K, Wlaschek M, Hogg A, et al. UVA irradiation induces collagenase expression in human dermal fibroblasts in vitro and in vivo. Arch Dermatol Res 1991;283:506-11

Petersen M J, Nasen C, Craig S, Ultraviolet A irradiation stimulates collagenase production in cultured human fibroblasts. J Invest Dermatol 1992;99:440-4

Jimenez S A, Derk C T. Following the molecular pathways toward an understanding of the pathogenesis of systemic sclerosis. Ann Intern Med 2004;140(1):37-50

Grewe M, Gyufko K, Krutmann J. Interleukin-10 production by cultured human keratinocytes: regulation by ultraviolet B and ultraviolet A1 radiation. J Invest Dermatol 1995;104:3-6

Morita A, Grewe M, Grether-Beck S, Olaizola-Horn S, Krutmann J. Induction of proinflammatory cytokines in human epidermoid carcinoma cells by in vitro ultraviolet A1 irradiation. Photochem Photobio In Press

Breuckmann F, Stuecker M, Altmeyer P, Kreuter A. Modulation of endothelial dysfunction and apoptosis: UVA1-mediated skin improvement in systemic sclerosis. Arch Dermatol Res 2004;296:235-9

Wlaschek M, Heinen G, Postwig A, Schwarz A, Krieg T, Scharffetter-Kochanek K. UVA-induced autocrine stimulation of fibroblast-derived-collagenase/MMP-1 by interrelated loops of interleukin-1 and interleukin-6 Photochem Photobiol 1994;59:550-6

Gruss C, Reed J A, Altmeyer P, McNutt N S, Kerscher M. Induction of interstitial collagenase (MMP-1) by UVA1 phototherapy in morphea fibroblasts. Lancet 1997;350:1295-6

### Customization and Measurement

The preferred embodiments of the present invention incorporates any suitable means to provide methods of patient customization into the treatment including, but not limited to, the method of automated testing, the recording and the analyzing of standard measurements of cell types, and cell type ratios, to optimally control the phototherapy for a given patient. Further, the preferred embodiments of the present invention incorporates any suitable biosensor means to provide the useful method of patient identification, and link the patient to the phototherapy prescription. Further, the preferred embodiments of the present invention make use of lab results including, but not limited to, blood analysis results, and other lab results. Wherein said lab results are input as data to any suitable phototherapy control means to provide the useful method of tracking the phototherapy progress, and to provide a feedback control signal to the phototherapy device for optimal approximation of a phototherapy prescription customization control for a given patient. Wherein said phototherapy prescription customization control incorporates phototherapy parameters including, but not limited to, spatial, temporal, and spectral variables. Wherein said lab results is comprised of measurement results including, but not limited to, T-cell populations, B-cell populations, natural killer (“NK”) cells, monocytes, dendritic cells, lymphoid dendritic cells, myeloid dendritic cells, T-cell sub-populations, membrane expression of molecules, B-cell specific chemicals, B-cell complement, intracellular cytokines of helper T-cells, mononuclear cells, monoclonal antibodies against various cytokines of interest, percent cytokine-positive CD4+ or CD8+ T-cells and any associated ratios, ratios of IFN-gamma to IL-4 cells for both positive and negative cell types in both CD4 and CD8 cells, IgG-C1q and IgG-C3 immunocomplexes using ELISA, ESR, CRP, TNA-alpha, IL-lbeta, IL-6, IL-8 and IL-12, and ICK plasma levels.

Wherein the measurement result for T-cell population is derived from lab tests for cell markers including, but not limited to, CD3+. Wherein the measurement result for B-cell populations is derived from lab tests for cell markers including, but not limited to, CD19+. Wherein the measurement result for NK cells populations is derived from lab tests for cell markers including, but not limited to, CD56+. Wherein the measurement result for monocytes population is derived from lab tests for cell markers including, but not limited to, CD 14+. Wherein the measurement result for dendritic cells population is derived from lab tests for cell markers including, but not limited to, CD123. Wherein the measurement result for lymphoid dendritic cells population is derived from lab tests for cell markers including, but not limited to, DR, and BDCA2+. Wherein the measurement result for myeloid dendritic cells population is derived from lab tests for cell markers including, but not limited to, CD11c, DR, BDCA1 and CD19−. Wherein the measurement result for membrane expression of molecules population is derived from lab tests for cell markers including, but not limited to, CD3, CD4, CD8, CD11c, CD14, CD16, CD19, CD56, alpha TCR, beta TCR, gamma TCR, delta TCR, CD123, HLA types, BDCA-1, and BDCA-2. Wherein the measurement result for intracellular cytokines of helper T-cells is derived from lab tests for cell markers including, but not limited to, IFN-gamma for T*SUB*H1 cells, and IL-4, IL-5 and IL-13 for T*SUB*H2 cells. Wherein the measurement result for B-cell specific chemicals is derived from lab tests for cell markers including, but not limited to, using IgG, IgA, IgM, C3, and C4. Wherein the measurement result for B-cell complement is derived from lab tests for cell markers including, but not limited to, IgG, IgA, IgM, C3, and C4. Wherein said T-cell sub-populations is comprised of T-cell with attributes including, but not limited to, T-cell receptors (“TCR”), alpha TCR, beta TCR, gamma TCR, and delta TCR. Wherein the measurement result for alpha TCR T-cell sub-population is derived from lab tests for cell markers including, but not limited to, CD3+. Wherein the measurement result for beta TCR T-cell sub-population is derived from lab tests for cell markers including, but not limited to, CD3+. Wherein the measurement result for gamma TCR T-cell sub-population is derived from lab tests for cell markers including, but not limited to, CD4+, and CD8+. Wherein the measurement result for delta TCR T-cell sub-population is derived from lab tests for cell markers including, but not limited to, CD4+, and CD8+.

The preferred embodiments of the present invention provide the useful method of providing a phototherapy to patients, by optimizing the approximation of a phototherapy prescription incorporating any suitable means capable of providing the methods to measure, record, and analyze the patient's response to a prescribed phototherapy. The directed application delivery means of the therapeutic light for therapeutic methods incorporates an initial standardized baseline phototherapy treatment encompassing the same combination or type of light to each patient with a specific disease. The initial standardized baseline treatment is then adapted for each patient on a patient-by-patient basis, by controlling the phototherapy customization variables means. Standard measurements are made of the concentration of one or more cell types, ratio of said target cells, number of said target cells clinical response of a specific tissue to specific spectral irradiance and to specific dynamic spectral irradiance or other values derived from measurement of tissue including, but not limited to, serum, and blood. Said standard measurements are used to track a patient's specific response to the phototherapy treatment. The measurement and customization procedures make use of historical data from previous patient optimization data in order to increase the accuracy of phototherapy optimization for a new patient's initial baseline phototherapy prescription. Said historical data is made anonymous to comply with the locales rules and regulations including, but not limited to, the Health Insurance Portability and Accountability Act (“HIPAA”). Said historical data is a combination of one or more data sets from test and measurement results including, but not limited to, genetic markers, previous lab results used to track illness progression, response to phototherapeutic wavelengths, and response to dynamic spectral irradiance. Measurement will preferably also include the error of observation for the data collected when available. Results and phototherapy optimization control signals are derived from data collected from said patient customization means, the use of predetermined optimized control algorithms and historical documentation, a patient's therapy is subsequently tailored to the patient's needs by implementing combinations of one or more phototherapy controls adjustments including, but not limited to, adjustment in the flux of light provided to that patient over a given period of time, adjustment to the length of time, and adjustment in the combination of sequenced spectral irradiance prescribed. Said phototherapy optimization control means provide combinations of one or more methods including, but not limited to, recording, tracking, analyzing, controlling, feedback control, feed-forward control, control optimization, allows the determination of the degree to which the therapy affected the patient on a combination of one or more phototherapy result variables. Wherein said phototherapy result variables are comprised of combinations of one or more result variable including, but not limited to, the number of cells modified, and the specific cell type ratios. The useful method of said phototherapy optimization control means allows the prescribing physician the capability to better understand how the patient's disease is responding to the treatment. The useful method of said phototherapy optimization control means allows the prescribing physician the capability to better understand how at least one group of patients with the disease are responding to the treatment. The useful method of said phototherapy optimization control means allows the prescribing physician the capability to better understand how the patient's clinical response compares to at least one group clinical responses, for the useful method of providing feedback control information to the phototherapy optimization control means and providing the initial conditions for the first set of phototherapy prescriptions.

The preferred embodiment of the present invention incorporates any suitable means including, but not limited to, cell testing sensors, that are capable of providing the useful methods including, but not limited to, cell testing for various cell types, and cell component testing for various cell components. Said cell testing means is any suitable means useful in providing feed-forward control schemes and initial phototherapy prescription estimation. Said cell testing sensors include, but not limited to optical sensors to analyze blood. Wherein said optical sensors include, but is not limited to, optical sensor adapted for sensing blood vessels visible in an eye and analyzing the blood within the blood vessels of an eye. Within an eye it is possible to apply a testing device comprised of testing device components including, but not limited to, retinal artery optical sensor means, to analyze the blood within the eye blood vessels including, but not limited to, the retinal arteries. Retinal arteries are visible through the pupil using said optical sensors means. Said retinal artery optical sensor means provides the useful method of phototherapy control for patient phototherapy optimization. Multiple individual patient data is combined to produce group results.

An optional feature of the preferred embodiments of the present invention is the use of injected chemicals including, but not limited to, immunofluorescent dyes, fluorescent dyes, contrast dyes injected into the blood or other tissue to help assess, test, and measure the phototherapy effects on tissue and specific cellular components. The injected chemicals bind the cellular component of interest and allow for increased or improved visualization of the phototherapy effects. The useful benefit of incorporating injected chemicals is to have a less invasive means of measuring cellular components and to assess patient clinical response to therapies.

Cellular and Tissue Components That EMR Targets

Cellular Targets

The following listed cell types are the major cell type classifications that are targeted by the preferred embodiments of the present invention. Whether a cell sub category is listed or a cell category is not listed, said target cell types are understood to include all sub-categories of the target cell type classifications listed. Well-known sub-categories of listed classes are included by reason of common knowledge. Whether listed or not listed, said target cell types include all variations of the cellular classifications listed, including, but not limited to, Lymphocytes, T-lymphocyte, T*SUB*H cell, T*SUB*C cell, T*SUB*H1 cell, specific tissues that have susceptibility to light, and organs that have susceptibility to phototherapeutic light. Wherein said phototherapeutic prescription is delivered by susceptibility of said target cells to the specific dynamic spectral irradiance of the light emitted by the preferred embodiments of the present invention. Said target cells are cells including, but not limited to, activated cells, inactivated cells, cells that have undergone a pathological change, cells that have undergone a genetic change, cells that have undergone a metabolic change, other changes not listed here including, but not limited to, certain cancers, and pathogen-altered cells. Wherein said pathogen altered cells are altered in various ways including, but not limited to, an infection with combinations of one or more specific pathogens including, but not limited to, bacteria, and virus. Said pathogen-altered cells are altered in ways that allow the preferred embodiments of the present invention to affect the pathogen-altered cell. Said target cells include cells not altered by a pathogen but also any other cell that is or is not functioning at what is considered a normal functional level for that specific cell type. Said pathogen altered cells include combinations of cell alterations including, but not limited to, metabolic alterations, secretory alterations, impaired functions, and altered functions. Wherein said metabolic alterations including, but not limited to, activated lymphocytes. Wherein said secretory alterations are combinations of one or more alterations including, but not limited to, normal, pathologic changes to a cell that alters the cells normal secretory product(s). Wherein said impaired function are combinations of one or more functions including, but not limited to, phagocytic cell unable to perform phagocytosis, secretory cell unable to secrete product. Wherein said altered function are combinations of one or more alterations including, but not limited to, normal cell becoming cancerous through some means and secreting enzymes to penetrate tissue and allow for the spread or metastasis of the cancer.

Said target cells are comprised of combinations of one or more target cell types, including, but not limited to, an infectious target type, and other target type.

Wherein said target cells are combinations of one or more target cell types including, but not limited to, leukocytes, plasma cells, macrophages, mast cell, dendritic cells, neuronal cells, neurons, epithelial cells, osteoblasts, endothelial cells, erythrocytes, hair follicles, dandruff, pet dander, lymphoid cells, myocytes, keratinocytes, melanocytes, stem cell, malignant cells, and muscle cells.

Wherein said leukocytes are combinations of one or more, white blood cell types including, but not limited to, lymphocyte, neutrophil, monocyte, eosinophil, basophil.

Wherein said lymphocytes are combinations of one or more cell types including, but not limited to, B-lymphocyte, and T-lymphocyte.

Wherein said B-Lymphocyte type includes all varieties and states of B-lymphocytes. Wherein said T-Lymphocyte type includes all varieties and states of T-lymphocytes.

Wherein said monocyte type includes all varieties and states of monocyte types.

Wherein said plasma cell type includes all varieties and states of plasma cells.

Wherein said macrophages types including, but not limited to, intestinal macrophages, alveolar macrophages, histiocytes, kupffer cells, mesangial cells, microglial cells, and osteoclasts.

Wherein said histiocytess are found in body components including, but not limited to, in the connective tissue.

Wherein said histiocytes are found in body components including, but not limited to, in the liver.

Wherein said mesanginial cells are found in body components including, but not limited to, in the kidney.

Wherein said microglial are found in body components including, but not limited to, in the brain.

Wherein said osteoclasts are found in body components including, but not limited to, in the bone.

Wherein said erythrocytes includes combination of one or more variety of erythrocyte cells including, but not limited to, red blood cells varieties, platelets. Wherein said pet dander target is a combination of one or more danders including, but not limited to, dog dander, and cat dander. Wherein said stem cell target is a combination of one or more target type including, but not limited to, and differentiated stem cells.

Wherein said muscle cell type is a combination of one or more cell types including, but not limited to, skeletal muscle cells, smooth muscle cells, cardiac muscle cells, striated muscle cells. Wherein said striated muscle cell type is a combination of one or more cell types including, but not limited to, striated muscle cell tissues, and striated muscle cell fibers.

Wherein said infectious targets include combinations of one or more target including, but not limited to, parasites, bacteria, fungi, and viruses.

Wherein said other targets include combinations of one or more targets including, but not limited to, proteins, chemicals, antigens/antibodies, DNA, RNA, nucleus of a cell, mitochondria, platelets, bone marrow, whole organs, glands, nodes, collagen, cartilage, connective tissue, fluid within a space, mitochondrial diseases, matrix metalloproteinase (“MMP”) and ensepholopathy.

Wherein said nodes target type is a combination of one or more targets including, but not limited to, a lymph node.

Wherein said fluid within a space is a combination of one or more body spaces including, but not limited to, abdomen, pleural space, ventricles of the brain, spinal cord fluid, cerebrospinal fluid, and synovial fluid in a joint.

Diseases, Disorders, Procedures or Other Pathological Processes

The preferred embodiments of the present invention incorporate any suitable means to provide useful therapy methods including, but not limited to, at least one of a phototherapy methods for a combinations for at least one of a target disease. Wherein said target diseases are combinations of one or more diseases including, but not limited to, SLE, an autoimmune disorder, a cutaneous disorders, a gastrointestinal disorders, a male disorders, a female disorders, a bladder diseases, a multiple sclerosis disease, a mood disorders, a circadian disorders, a blood disorders, a bone marrow borne diseases, a malignancy, a breast cancer, a lung cancer, a transplant tissue, a graft tissue, a viral diseases, other indications, a cutaneous T-lymphocyte lymphoma, an allergic reactions, dandruff, alopecia, polycystic ovarian syndrome (“PCOS”), a blood based parasite diseases, a tissue based parasite diseases, a viral diseases, a bacteria diseases, a fungi diseases, a diseases of the immune system, asthma, a pre-natal disease, a pre-term disease, congenital infections, a hematological disease, a newborn diseases, asthma, atopy, IgA hypersensitivity, and cystic fibrosis.

Wherein said target diseases autoimmune disorders are combinations and variations of diseases including, but not limited to, rheumatoid arthritis, ankylosing spondylitis, polymyositis, dermatomyositis, myositis, systemic sclerosis, scleroderma, Hashimoto's thyroiditis, Grave's disease, and any other disease involving improperly activated immune system and/or the production of antibodies that either decrease the activity of an organ, tissue or cell type or increase cell activity resulting in a pathological process or a disease where stimulation of the immune system increases production of antibodies and cell types and cytokines to increase stimulation of an organ, and tissue or cell for a therapeutic method.

Wherein said target diseases cutaneous disorders are combinations and variations of diseases including, but not limited to, psoriasis, albinism, xeroderma pigmentosa, acne, and vitiligo.

Wherein said target diseases gastrointestinal disorders are combinations and variations of diseases including, but not limited to, Crohn's disease, ulcerative colitis, inflammatory bowel disease, diverticulosis, and diverticulitis.

Wherein said male diseases are combinations of one or more diseases including, but not limited to, urogenital disorders, male reproductive disorders, benign prostatic hyperplasia (“BPH”), and prostate cancer.

Wherein said female diseases are combinations of one or more diseases including, but not limited to, urogenital disorders, and female reproductive disorders, gynecological diseases, vaginal diseases, uterine diseases, cervical diseases, fallopian tube diseases, ovarian disease, polycystic ovarian syndrome, other female diseases, uterine fibroids, hydatidiform mole, endometriosis, myometritis, cervical cancer, dysplasia of female-specific tissues, uterus malignancy, ovarian malignancy, vaginal malignancy, and female anatomy malignancy.

Wherein said target diseases mood disorders are combinations of one or more diseases including, but not limited to, seasonal affective disorder (“SAD”), sleep irregularities, insomnia, daytime somnolence, sleep cycle irregularities, depression, and bipolar disorder.

Wherein said target diseases circadian disorders are combinations of one or more diseases including, but not limited to, seasonal affective disorder (“SAD”), sleep irregularities, insomnia, daytime somnolence, sleep cycle irregularities, depression, and bipolar disorder.

Wherein said target diseases blood disorders are combinations of one or more diseases including, but not limited to, malaria, HIV/AIDS, Lymphoma, Leukemia, any blood, tissue based parasite, virus, bacteria, fungi or other organism, any other disorder within the bone marrow or blood that will benefit from the modulation and alteration of populations or activities of various cell populations using light.

Wherein said target diseases bone marrow borne diseases are combinations of one or more diseases including, but not limited to, malaria, HIV/AIDS, Lymphoma, Leukemia, any blood, tissue based parasite, virus, bacteria, fungi or other organism, any other disorder within the bone marrow or blood that will benefit from the modulation and alteration of populations or activities of various cell populations using light.

Wherein said target diseases malignancies are combinations of one or more diseases including, but not limited to, glioblastoma, neuroblastoma, solid tumors, malignancies, cutaneous tumors, and systemic tumors.

Wherein said target diseases transplant tissue are combinations of one or more diseases including, but not limited to, GVHD. Whereby a method of use is made of electromagnetic radiation to pre-treat graft tissue and decrease the number of cells within the graft that could react inside the host once the graft is placed, immune modulation, immune stimulation, bone marrow transplants (“BMT”). Whereby a method of use of the preferred embodiments of the present inventions therapeutic light to pre-treat the bone marrow before it is placed in the host to decrease the reactive cells and the potentially reactive cells, such as in diseases including, but not limited to, GVHD, which can occur with a BMT.

Wherein said target diseases graft tissue are combinations of one or more diseases including, but not limited to, GVHD. Whereby a method of use is made of electromagnetic radiation to pre-treat graft tissue and decrease the number of cells within the graft that could react inside the host once the graft is placed, immune modulation, immune stimulation, BMT. Whereby a method of use of the preferred embodiments of the present inventions therapeutic light to pre-treat the bone marrow before it is placed in the host to decrease the reactive cells and the potentially reactive cells, such as in diseases including, but not limited to, GVHD, which can occur with a BMT.

Wherein said target diseases viral diseases are combinations of one or more diseases including, but not limited to, double-stranded DNA viruses, single-stranded DNA viruses, double-stranded RNA viruses, single-stranded RNA viruses, and Epstein-Barr virus.

Wherein said target diseases are combinations of one or more diseases including, but not limited to, disease pre-term disease are combinations and variations of diseases including, but not limited to, Rh disease.

Wherein said post newborn diseases are combinations of one or more diseases including, but not limited to, typical 6 months after birth, infections, immunodeficiencies of the pediatric population, childhood autoimmune diseases, Sturge-Weber and port-wine diseases, transplant patients, kidney, and bone-marrow.

Wherein said target diseases are combinations of one or more diseases including, but not limited to, diseases of the immune system, asthma, and cystic fibrosis.

Wherein said autoimmune disorders are immune conditions including, but not limited to, an improperly activated immune system, inactive lymphocytes requiring a need to stimulate lymphocytes, active lymphocytes requiring a need to eliminate lymphocytes. When there are too many lymphocytes the advantage of eliminating lymphocytes including, but not limited to, certain harmful antibodies are not generated, inflammatory processes are down-regulated and/or immune complexes are not formed, and there is reduced improper tissue destruction or inflammation. Autoimmune disorders requiring down-regulation is a combination of effects including, but not limited to, an indirect or direct reduction in the production of inflammatory cytokines, a decrease in activation of additional lymphocytes of all types, and specific aspects of an immune response. The effects are referred to as a modulation of the immune system in order to derive a positive benefit.

Wherein said autoimmune disorders with autoimmune disorder conditions have conditions including, but not limited to, an improperly activated immune system, a need to stimulate a first set of disease specific lymphocytes, a need to eliminate a second set of disease lymphocytes. The advantages of eliminating lymphocytes include, but are not limited to, certain harmful antibodies are not generated, and inflammatory processes are down-regulated. Down-regulation is a combination of effects including, but not limited to, an indirect reduction in the production of inflammatory cytokines, a direct reduction in the production of inflammatory cytokines, a decrease in activation of additional lymphocytes of all types, and specific aspects of an immune response. Down-regulation also includes a decrease in the damage that is mediated by immune complexes and auto-antibodies that bind to tissue. The phototherapeutic effects, including, but not limited to, the down-regulation effect are also known as a modulation of the immune system in order to derive a positive benefit.

Wherein said immune modulation is a combination of one or more prescription types, including, but not limited to, a general prophylactic prescription, and a specific prescription targeting a specific set of target diseases. In the specific prescription for a transplant patient, wherein use is made of one of a dynamic spectral irradiance including certain phototherapeutic wavelengths or certain discrete wavelengths of directed application of light either on the implanted graft or externally to the whole body of the transplant patient to down-regulate the immune system and reduce and/or eliminate any cells that are involved in the potential rejection of the graft, such as naive lymphocytes, activated lymphocytes, activated T-foreign lymphocytes, and activated native lymphocytes involved in cell-mediated rejection of a graft, including, but not limited to, acute, chronic, or any other rejection at any point after the graft is in place, including GVHD. The phototherapeutic effect on GVHD is accomplished by a similar mechanism as for SLE as the cells involved in graft rejection are also located in the blood and are activated.

Wherein said allergic reactions including, but not limited to, dust or pollen allergies, and allergic reactions mediated by cells including, but not limited to, B-lymphocytes, mast cells and plasma cells, wherein the preferred embodiments of the present invention is used to decrease the allergic reaction immune response and/or histamine release and the release of any other chemicals released in the allergic reaction immune response.

Wherein said immune stimulation by some wavelengths of light increase the number of beneficial cells in the immune system, the use of UV light boosts the beneficial aspects of the immune system to help prevent infection. Often, the immune system of a transplant patient needs to be suppressed to prevent rejection of the graft, in which case the immune suppression capabilities of certain undesirable wavelength ranges, certain undesirable wavelengths and certain undesirable sequences of spectral irradiance flux are to be avoided.

The various embodiments of the present invention are better suited to meet said target disease specific phototherapy prescription specifications by the incorporation of any suitable means including, but not limited to, UV-LEDs, UVA-LEDs, and UVA1-LEDs.

LEDs have the capability to deliver a specific wavelength range of light that is desired for a specific type of phototherapy for which each wavelength, the aggregate collection of wavelengths, and the phototherapy dynamic spectral irradiance, has been shown to have specific phototherapy properties for a given flux over a given time. LEDs are responsive to an electric current control to closely follow a dynamic radiant flux output over a period of time to accurately deliver directed application within response times shorter than typical fluorescent bulbs. UV-LEDs are unlike any other prior-art light sources in that LEDs have the capability to deliver a controlled and specific amount of UV light substantially without any amount of UVB or UVC light. LEDs have a recognizable difference and variations from any non-LED light source available for phototherapy, wherein said non-LED light sources are unable to eliminate incidental light of the UVB or UVC type. UVA2, UVB and UVC have been shown to have substantial damaging properties to tissue, skin, organs, and cells and should not be present for the methods of, UVA skin tanning, UVA phototherapy, UVA1 phototherapy, and UVA1C phototherapy. The reduction of the risk of exposure to stray UVB, UVC, and UVA2 by the preferred embodiments of the present invention provides a useful improvement over the prior art phototherapies.

The preferred embodiments of the present invention incorporates any suitable means to measure the phototherapy spectral irradiance characteristic flux and thereby generate an output signal indicating the values of the spectral irradiance measurement signal. The preferred embodiments of the present invention incorporates any suitable means responsive to said spectral irradiance measurement signal capable of controlling the power to said phototherapeutic LED array to effectively optimize the close approximation of the phototherapy prescription, and minimize the difference between the phototherapy prescription and the actual phototherapy. The preferred embodiments of the present invention further incorporates any suitable spectral irradiance measurement means to record the actual difference between the phototherapy prescription and the prescribed prescription for use in modifying subsequent phototherapy sessions to compensate for the effects said actual difference may create that need to be addressed in a subsequent session. Further, the preferred embodiments of the present invention incorporates any suitable means to measure UVB and any suitable means to measure UVC, to provide the method of activating a safety cutoff function to prevent over-exposure to UVB and to UVC. Said spectral irradiance measurement means include any suitable means capable of measuring flux wavelength ranges and specific wavelengths devices including, but not limited to, spectroradiometer, spectrometer, prism, diffraction gratings, optical charge coupled sensors, photo-sensitive film, slits, mechanical tuning, and computer controlled tuning, and blazed diffraction gratings, Wherein said spectrometer is selected from available types including, but not limited to, Edmund Optics type UV CCD Spectrometer with a Edmund Optics Stock No. J57-053 as described in the “2007 Optics and Optical Instruments Catalog” with a catalog number N078A.

The external form factors of the preferred embodiments of the present invention incorporate specific phototherapeutic LED types to deliver phototherapeutic light to the skin to penetrate to said blood in the blood vessels of the skin to provide phototherapeutic effects including, but not limited to, control various cell populations within the blood that respond to dynamic spectral irradiance flux comprised of combinations of one or more phototherapeutic light including, but not limited to, UVA, UVA1, UVA1C, discrete wavelength(s), and in combination with other suitable light sources. Wherein said control of various cell populations is accomplished by combinations of one or more phototherapeutic effects including, but not limited to, down-regulate the T-lymphocytes of the T*SUB*H1 type, up-regulate the T-lymphocytes of the T*SUB*H2 type. Wherein said control of various cell concentrations serves the useful method of providing a therapy for diseases including, but not limited to, SLE. The use of combinations of one or more phototherapeutic UV-LEDs, UVA-LEDs, UVA1-LEDs, and UVA1C-LEDs also promotes a photo-protective tan through the skin-tanning process. Wherein said phototherapeutic LEDs, are combinations of one or more LEDs with peak wavelengths including, but not limited to, UVA1, UVA1C. UV-LEDs offer a distinct advantage over other light sources. Since the UVA1-LEDs substantially do not emit UVA2, UVB or UVC light, and the UVA1-LED allow the control of the range of light necessary to provide the phototherapy including, but not limited to, UVA1 SLE phototherapy, and said target disease phototherapies. The presence of UVB light in prior art light sources is a cause of harm to SLE patients as only UVA1 light has been shown to be therapeutic to SLE patients. A filtered fluorescent lamp based prior art phototherapy has been documented in the medical literature, but the prior art phototherapy has not disclosed nor anticipated any of the benefits of LEDs to deliver the desired wavelengths of light. The prior art phototherapy have not disclosed the ability to dynamically manipulate the spectral irradiance of the phototherapy. The prior art does not disclose the use of LEDs in a manner that increases the probability of certain chemical reactions that increase the effectiveness of the phototherapeutic effects. The prior art does not disclose the capabilities of controlling the lamp temperature to effectively control the spectral irradiance. The preferred embodiments of the present invention make use of one or more LED types of varied spectral irradiance to sequence wavelength ranges to increase the capability to optimize the approximation of a phototherapy prescription. In the preferred embodiments of the present invention the varied spectral irradiance is purposefully manipulated by controlling the temperature of the semiconductor junctions in the LEDs of the same type. In a preferred embodiment of the present invention the varied spectral irradiance is purposefully manipulated by controlling the temperature of the semiconductor junctions in the LEDs of a differing type. Said temperature controlled LEDs are combinations of one or more LED types or of the same type. LEDs in an LED array are preferably controlled independently for fine gradient temperature control and thus require a suitable amount of thermal insulation to increase the effectiveness of the independent thermal control regions. Wherein said thermal insulation is purposefully selected to not degrade due to UV light exposure.

The authors of scientific journal articles have had to “coin” new terms to describe other specific UV ranges. The term UVA1 and UVA2 have been used, which reflects the science and medical communities need to have a higher degree of specificity in the discussion of the UV effects. These additional definitions are still not sufficient to properly describe the capabilities of the LED light sources. LED manufacturer typically provide data sheets on each LED type that includes a chart of the relative irradiance over wavelength at a specific temperature and forward current. A common way to describe LED type is by two typical LED characteristics derived from the spectral irradiance chart of relative irradiance over wavelength. The two derived LED characteristics are known as the peak wavelength and the full width half maximum (“FWHM”). The spectral irradiance chart of relative irradiance versus wavelength more accurately describes the characteristics of a given LED types nominal characteristics compared to stating the LEDs peak wavelength and FWHM. A typical LED has a spectral irradiance that varies with forward current, and the forward current is purposefully controlled to manipulate the spectral irradiance to closely approximate a phototherapy prescription. When the LED current required to shift a spectral irradiance is higher than the average sustainable current rating of the LED type, then the LEDs are pulsed with a fractional duty cycle to allow for the LED to cool before the next pulse. When a continuous non-pulsed output is required in the phototherapy then multiple LEDs are sequentially pulsed at a time inter-leaved basis to simulate a constant output from the time inter-leaved LED array. A time inter-leaved array incorporates any suitable means capable of providing the useful methods of distributing the light in a spatially consistent manner including, but not limited to, a diffuser, a lens, and a collimator.

A primary mechanism for the acceleration of scientific knowledge for UV related studies is the ongoing development of ultraviolet light-emitting diode (“UV-LED”) technology. UV-LEDs provide a fundamental change in the way UV studies are implemented, primarily due to the increase in control that LEDs offer over incumbent light sources. UV-LEDs have increased flux spectral, temporal, and spatial resolutions over incumbent UV light sources. The method of using of LEDs to increase control over multiple variables offers the opportunity to design experiments that would be impractical, if not impossible, to conduct with incumbent light sources. Incumbent light sources are also known as legacy light sources.

A distinguishing feature of LED light sources is that the overall spectral irradiance output is continuous over a wavelength range. A typical LED has substantially no fixed discrete spectral lines as compared to a mercury vapor discharge lamp. The typical LED characteristically has at least one peak wavelength. Wherein said LED peak wavelength is typically dependent on the LED semiconductor junction temperature gradients and the LED junction current gradients. The LED typically has a bell shaped curve for the chart of relative spectral irradiance. The LED characteristic full width half maximum is defined as the width in nanometers of said bell shaped curve at 50% of the peak wavelength. LEDs are incorporated into the preferred embodiments of the present invention that also exhibit multiple peaks in the chart of spectral irradiance. The LED characteristic of peak wavelength is less useful for describing LEDs that have more than one significant peak and in this case the average wavelength and the standard deviation is used to characterize the LED, instead of the FWHM and peak wavelength. Multiple LED types with various peak wavelengths are incorporated in the preferred embodiments of the present invention to create a dynamic spectral irradiance that optimizes the phototherapy prescription. Since, the spectral irradiance of the Sun is modeled as a hot black body radiator with a highly continuous spectral irradiance distribution, LEDs are a closer match to the output of the Sun than any other legacy bulb type. Therefore, it is the LEDs similarity to solar emissions that allows for phototherapies that are a closer match to the natural conditions under which humans adapted and is useful in the preferred embodiments of the present invention. The closer match to natural evolutionary conditions is useful since there is less of a probability that any LED phototherapy patient will experience an unforeseen reaction due to conditions to which the species has not adapted. Multiple LEDs with varying peak wavelengths are combined to simulate the terrestrial spectral irradiance of the Sun more closely than incandescent lamp types, since there are low points in the terrestrial sunlight spectral irradiance that do not exist in incandescent bulbs. A combination of one or more LED array types, wherein each array is comprised of a combination of one or more LED types, has the capability to dynamically control temporal, spatial and spectral irradiance, and therefore are more suitable phototherapy for biological experiments than incandescent lamp types. Fluorescent bulb types generally have ballasts creating a pulsed high-voltage useful for power conversion pulsed emission. The pulsing fluorescent light is due to the ballast electrical supply which has a pulse frequency and current associated with converting a mains electrical supply of typically 115 volts AC to 230 volts AC to a high voltage needed to arc through the fluorescent tube. Said fluorescent ballast power conversion pulsed emission is not optimized for phototherapeutic effect and is not related to the improvement disclosed herein. The improved phototherapy means and methods disclosed herein is capable of purposefully pulsing the phototherapeutic LEDs in a dynamically powered manner to increase the probability of specific chemical reactions leading to biological effects. LEDs have the capability to be pulsed to provide accurate dynamic pulses of phototherapeutic light to minimize the difference between a prescribed phototherapy and the actual phototherapy delivered.

Because the wavelengths in an LED lamp are purposefully selected, and purposefully controlled by manipulating environmental controls, and active operating conditions, whereby the preferred embodiments of the present invention have substantially no harmful UVB and have substantially no harmful UVC spectral irradiance components.

The use of LED technology provides the preferred embodiments of the present invention with a new and useful improvement over the prior art due to an increase in the control of the flux of light, and in programmable sequencing of dynamic spectral irradiance densities, and in dynamic spatial densities, which are not possible using mercury vapor emission bulbs and filters, and spectral power densities.

Like the natural rays from the sun, LED's have a spectral irradiance that is continuous across the range of emitted wavelengths and compare favorably to the high peak spectral irradiance flux at fixed discrete spectral lines present in the legacy mercury vapor emission based lamps. An additional improvement over prior art offered by the preferred embodiments of the present invention is that LEDs and LED power supplies are well suited to continuously compensate for LED aging by adjusting the forward current supplied to the LED to promote increased accuracy for a prescribed phototherapy as the LEDs age. A spectrometer is incorporated into the preferred embodiments of the present invention providing the capability to measure the spectral irradiance data used to calculate the compensation adjustments of a substantially constant flux. The method of performing an initial calibration of the spectrometer to the phototherapy device, allows the LED power supply to make real time adjustments to minimize the difference between the current spectrometer measurement and the initial measurement.

LEDs are preferably packaged in modular low thermal resistance packages for the useful method of increasing ease of maintenance and the ability to quickly change the phototherapeutic capabilities of the preferred embodiments of the present invention. Multiple LED types are incorporated and selectively enabled to provide a multi-use phototherapy chamber capable of treating multiple different disease indications without taking up additional facility space in a medical office.

LEDs are selected for use in the preferred embodiments of the present invention that emit specific ranges of UV depending on requirements in temporal, spatial and spectral irradiance variables, and are also selected that do not emit specific ranges of light depending on requirements in temporal, spatial and spectral irradiance variables. The selection method of LEDs allows for a customization of the phototherapy session for different skin types, and disease indications. LEDs that are manufactured have varying characteristics depending on type. Wherein the LED type characteristics include, but are not limited to, LED type specific spectral irradiance, and LED type aging characteristics. Said LED specific spectral irradiance is a function of LED age and history of use. Said recorded history of use will be made in a fashion suitable for computer algorithm analysis for control system methods to optimize the accuracy of delivering a phototherapy prescription. The history of the LED will be recorded and used for deployment optimization controls. More than one type of UVA1-LED exist. Some UVA1-LED may contain trace amounts of higher energy photons of type UVB and UVC, which requires to be used in combination one or more optical filters including, an optical spectral low-pass filter, and an optical spectral band-pass filter. Some LED types may require a filter, and other LED types may not require a filter to avoid the generation of the neutral wavelengths provides the useful improvement of increased energy efficiency and lower overall cost of providing the therapy. The preferred embodiments of the present invention make the use of filters optional as needed. The preferred mode of operation is use of UVA1-LED without significant UVA2, UVB, or UVC content and therefore without an optical spectral filter. However, optical spectral filters are optionally used to modify the spectral output of LEDs when the native spectral output from the LED needs additional contouring to more accurately meet at phototherapeutic prescription. The preferred embodiments of the present invention also incorporate suitable means to dynamically change the optical spectral filter from a set of optical spectral filters to optimize the accuracy of providing a phototherapy prescription. The preferred mode of operation has the benefit of operating without an optical spectral filter that provides the benefit of the most energy efficient mode of the phototherapy system.

In addition to the typical phototherapy bed form factor, the UVA1-LED technology can be incorporated in the flexible form factor of phototherapy clothes and in the form factor phototherapy of fabric to allow for special directed application including, but not limited to, a lower flux dose over a longer time period to be applied while sleeping, and a lower flux dose over a longer period of time while the patient is involved in other normal waking activities. The longer period of application of the phototherapeutic light provides the useful method of providing a phototherapy while allowing other typical or necessary activities. The flexible form factor of the preferred embodiments of the present invention provides useful improvements by increasing the availability and ease of use of the phototherapy, as there is reduced need to travel to a doctor's office or other phototherapy location to obtain the phototherapy. The preferred embodiments of the present invention incorporates any suitable means capable of providing the methods of remote access capabilities including, but not limited to, a physician to monitor the progress of a phototherapy as required by said phototherapy prescription, and regulations of phototherapy providers, even when administered remotely from a doctor's office. Other human compatible characteristics of the LED include, but are not limited to, a low voltage, near body temperature of operation, and reduced potential hazard of glass breakage.

### Medical Application

There are at least two categories of benefits from the use of UVA1 light as a treatment for SLE; the first category of benefit of the preferred embodiments of the present invention is the photo-protective barrier, and the second category of benefits systemic disease treatment. The following section elaborates on the mechanism of the two categories of benefits and how the two mechanisms combine to create a highly effective therapy.

### The Photo-Protection Mechanism

The photo-protection mechanism is based largely on the effects that UVA light is known to have with skin.

UVA and UVB light have a short-term effect on the skin, typically resulting in pigmentation from UVA, melanogenesis from UVB, erythema, and injury to the keratinocytes and Langerhans cells in the epidermis. The intensity of the light and length of exposure dictate the degree to which these effects are seen. UVA light is particularly important in that it induces the conversion of melanin, which causes an immediate darkening of the skin. Tanning by UVA, including, but not limited to, tanning by UVA1 light, promotes a variable protective barrier against further exposure to ultraviolet radiation without inducing an adverse reaction in phototherapy patient including, but not limited to, SLE patients. UVB and UVC light are known to cause significant increases in cutaneous and systemic symptomatology in SLE patients. Relevant and exemplary effects of UVA, UVB and UVC light are described herein. Wherein said variable protective barrier is also known as tanned skin. Wherein said tanned skin has characteristic effectiveness dependent on variables including, but not limited to, skin type, tanning history, and phototherapy history. Said skin tanning process occurs when photons of approximately UVA wavelength stimulates the conversion of melanin. Melanin concentration varies with skin type. Melanin is secreted from the melanocytes as a result of melanogenesis. UVB stimulates the melanocytes to increase the rate of melanogenesis relative to normal endogenous rate of melanogenesis present without UVB stimulation. A UVB photon creates a delayed reaction by activating the melanocytes to produce melanin at a higher rate than normal. However, in most of the population endogenous melanogenesis occurs without UVB stimulation at a quiescent rate. The quiescent rate of melanogenesis results in the endogenous melanin that is the normally occurring within skin melanocytes.

UVC Light

UVC is mostly attributed as having serious carcinogenic effects, and is responsible for both basal and squamous cell carcinomas, as well as melanomas.

UVC is the most energetic form of ultraviolet light and therefore the most dangerous. Even at very short exposure times, UVC poses a serious threat as a mutagenic and toxic light source.

UVB Light

UVB has acute effects related to the killing of keratinocytes and is attributed to a delayed effect of premature aging, actinic keratosis and skin cancer. UVB has a direct link in the literature to squamous cell carcinoma (“SCC”) and basal cell carcinoma (“BCC”).

Due to the genotoxic effects of UVB, UVB is approximately 1000 times more likely to cause erythema than UVA light.

UVB induces skin damage via the generation of reactive oxygen species and the damage of melanin, as well as damage to the DNA of the cells by the formation of pyrimidine dimers within the DNA strand.

The capability of skin to protect itself from free radicals generated as a result of exposure to sunlight is decreased due to a loss of antioxidants caused by UVB light exposure.

UVA Light

UVA has an effect of pigment darkening and resultant tanning with lesser link to the development of skin cancer. UVA1 can be orders of magnitudes less harmful than UVA3, UVA2, UVB and UVC

UVA penetrates the skin deeper than UVB and UVC, allowing a larger flux gradient through the skin depth of the initial portion of the incident flux to penetrate to the deep levels of the dermis and the epidermis. The penetration capabilities allow UVA to be effective at producing a tanning effect, causing a darkening of the melanin within the skin.

UVA photons are generally less reactive than either UVB photons or UVC photons. UVA photons have a higher probability of passing through the epidermis than UVB photons or UVC photons, and therefore UVA has a higher probability of reaching the dermis and of reacting with body components including, but not limited to, dermis, vascular components of the dermis, blood vessels, capillaries, and blood.

By providing the enhanced pigmentation generated by UVA light, without the comparably increased damaging effects of UVB and UVC light, the UVA1 therapy promotes a protective barrier that afford SLE patients an additional barrier of protection from natural sunlight.

The Systemic Mechanism

Because a UVA photon has a higher probability of penetrating deeper into the skin than UVB and UVC, UVA light has the capability to penetrate deep into the dermis and epidermis. UVA light can gain access to the target blood vessels, wherein said target blood vessels is comprised of combinations of one or more blood vessels. Wherein said blood vessels are carrying blood to and from the skin. Within said target blood vessels are responsible for carrying nutrients throughout the body as well as providing the body with some immune system components for immune protection against infection and injury. However, some of the cells that provide the immune protection are also part of the pathophysiology of lupus.

The capillary network present in the skin shuttles approximately 2-5 liters of blood every 10 minutes, for a total blood flow in the skin of 200-500 ml/min, allowing nearly complete photo-irradiation of the body's total blood supply in a reasonably short period of time compared to the duration of a typical phototherapy session. The cutaneous presentation of blood at a skin depth accessible by UVA light forms the basis for the induction of the systemic effects of UVA1 phototherapy.

The T-lymphocyte cell types and the B-lymphocyte cell types are both implicated in the pathophysiology of SLE. The T-lymphocyte types and the B-lymphocyte types are, in varying degrees, sensitive to irradiation by UVA1 light, undergoing apoptosis under certain conditions. Knowledge of the UVA1 effects on lymphocytes and knowledge of the blood flow to the skin led to the discovery that the predominant means of systemic treatment of SLE is achieved by a decrease in specific types of circulating T-lymphocyte and B-lymphocytes within the body. Both the action spectra for apoptosis of B-lymphocytes, and the action spectra for apoptosis of T-lymphocytes vary as a function of wavelength. The preferred embodiments of the present invention make use of the action spectra for apoptosis of T-cells to optimize the phototherapy accuracy of delivering the prescribed phototherapy. The preferred embodiments of the present invention make use of the action spectra for apoptosis of B-cells to optimize the phototherapy accuracy of delivering the prescribed phototherapy.

The CD4+ T*SUB*H1 T-lymphocyte is implicated in the success of the treatment of autoimmune diseases including, but not limited to, target diseases, and SLE. The IFN-gamma produced by the CD4+ T*SUB*H1 is part of the cause of the pathogenesis of the disease. Published research has demonstrated the success of the UVA1 phototherapy as it causes a decrease in the T*SUB*H1 cells, a relative increase in ratio of IL-4 secreting T*SUB*H2 cells to T*SUB*H1, and a decrease in the T*SUB*C2 cells. The preferred embodiments of the present invention incorporates any method to significantly reduce the T*SUB*C1/T*SUB*C2 ratio and reduce the T*SUB*H1/T*SUB*H2 ratio, which is one of the main mechanisms for phototherapeutic treatment provided by the preferred embodiments of the present invention.

The preferred embodiments of the present invention improves on prior art related to phototherapies, including, but not limited to, target disease phototherapies, SLE phototherapy; by more accurately and precisely controlling the spectral irradiance flux and temporal variables associated with delivering the phototherapy prescription, via directed application, of the most efficacious available wavelength range(s) of light in the correct spatial orientations to the body components for a prescribed period of time to enact the mechanisms described herein. Wherein said phototherapy prescription originates from one or more sources including, but not limited to, practicing physicians. Wherein said phototherapy prescription is programmed via prescription document technology including, but not limited to, personal smart card, token for database record, multi-factor authentication system, and ink on paper, and FDA modular label with label programming means. Wherein said phototherapy prescription is implemented by personal roles including, but not limited to, trained staff, and otherwise monitored remotely for proper operation.

The B-lymphocyte performs a critical function in disease processes in general, and in particular the disease processes of autoimmune diseases including, but not limited to, SLE, and similar autoimmune diseases including, but not limited to, said target diseases. Since certain B-lymphocyte types, including, but not limited to, the CD27 high plasma cell type, produces circulating antibodies, such as the anti-DNA antibodies of SLE. UVA1 phototherapies promote the controlled suppression of the activity of B-cells and/or induce apoptosis of these cells in blood vessels of the dermis. Following UVA1 irradiation, published SLE phototherapy research using prior art phototherapy devices indicates a reduction in the circulating antibodies in SLE patients. The preferred embodiments of the present invention offers an improvement over the prior art including, but not limited to, the reduction of the risk of exposure to stray UVB and UVC light, while targeting the more beneficial useful wavelength ranges and eliminating the neutral and the harmful wavelengths. In an effort to eliminate UVC and UVB light from lamps within the room and/or from the phototherapy equipment, one or more optical spectral filters is provided in certain installations requiring enhanced filtering in order to meet the requirements of the phototherapy prescription. Research has shown that the presence of B-lymphocytes is directly related to disease activity in SLE patients. UVA1 phototherapy decreases the number of circulating lymphocytes via apoptosis and contributes to the reduction of symptoms of diseases including, but not limited to, arthritis, leucocyturia/erythrocyturia, blood pressure, and myalgia/myositis. One of the ways UVA1 phototherapy decreases other symptoms is by decreasing immunoglobulin production and apoptosis of the B-cell. The preferred embodiments of the present invention incorporate any suitable means that provides the method of promoting the clearance of apoptosis fractions.

Recent data shows even more promise including, but not limited to, improvements in patients experiencing cognitive decline from SLE, is an additive effect of the treatment. Wherein said additives effect includes, but not limited to, gains from previous treatments enhance following treatments, and decreases in clinical indices of disease of up to 70% in some studies. In addition to this, one study indicated that the benefits from one low-dose UVA1 therapy have persisted for over three years.

To date, the preponderance of journal articles indicate that no toxicities from UVA1 phototherapy treatment or significant adverse effects have been found. The same reduction in toxicities is not possible with the current pharmacological therapies. Potential side effects of a UVA1 phototherapy that have been described in the literature are occasional redness and dry skin as well as an increase in the pigmentation of the skin. The pigmentation provides a variable photo-protective barrier against the harmful light of the sun and is more of a benefit than an adverse effect for many patients. Said photo-protective barrier varies from patient to patient and varies for each patient dependent on patient state. Said patient state including, but not limited to, phototherapy history, skin color, age, food, and drug intake. Said patient state is dynamic and thus varies over time. The various preferred embodiments of the present invention incorporate any suitable means capable of providing the useful method of processing patient state dynamically to optimize the accuracy of delivering a phototherapy prescription.

Additional phototherapy target types are comprised of combination of one or more target type including, but not limited to cellular targets, chemical targets, protein targets, and enzymatic targets. Wherein said chemical target types are target types including, but not limited to, inflammatory cytokines, ions, minerals, vitamins, vitamin precursors, vitamin metabolites, circulating heme, heme in red blood cells, heme in cells, bilirubin, bilirubin precursors, bilirubin conjugates, and bilirubin metabolites. Wherein said enzymatic target types are target types including, but not limited to, matrix metalloproteinases (MMPs). Wherein said MMPs are enzymes that function in connective tissue including, but not limited to, collagen, and elastin. MMPs perform functions on said connective tissue including, but not limited to, breakdown, and destruction, whereby MMPs contribute to skin and cartilage remodeling. Increased MMP activity is associated with some undesirable processes including, but not limited to, increased aging and fragility of skin. The preferred embodiments of the present invention incorporate any suitable means capable of providing the useful methods of UVA1 phototherapy capabilities including, but not limited to, down-regulating the effects of MMPs and said enzyme target types.

Macrophages originate from the white blood cell line of monocytes and serve multiple functions including, but not limited to, innate immunity, and cell-mediated immunity. Macrophage function in both innate and cell-mediated immunity by performing many processes including but not limited to phagocytosis. Phagocytosis is an immune process whereby the macrophage engulfs and breaks down cellular debris and/or pathogens. Macrophages also perform the role of an antigen presenting cell and thereby stimulate an immune response to specific pathogens from various immune cells, including but not limited to, lymphocytes. The role of macrophages in SLE disease processes has been described as ineffective since macrophages have diminished capability to clear cellular debris. Said cellular debris is comprised of combinations of debris including, but not limited to, the products of apoptosis, apoptotic bodies, apoptotic blebs, the products of necrosis, components on the surface of apoptotic blebs, components on the surface of apoptotic bodies, cell components that typically are protected within an intact cell, and other cellular products. One of the many mechanisms by which SLE manifests is due to the presence of un-cleared cellular debris over a period of time sufficient for the immune system to recognize the un-cleared cellular debris as foreign, whereby the immune system develops autoantibodies as the immune system reacts against lingering cellular debris. Under normal physiologic conditions, macrophages clear cellular debris within a short enough period of time to prevent the formation of autoimmunity.

The effects of the UVA phototherapy are combinations of one or more effect including, but not limited to, the effect of improving the function of macrophages in SLE, the effect of improved macrophage clearance of cellular debris, the effect of improved macrophage signaling capabilities, the effect of decreasing the amount of cellular damage caused by reactive-oxidant species resulting from autoimmune reactions due to antigens on lingering cellular debris. An additional disease mechanism by which SLE manifests is the deposition of immune-complexes in SLE resulting in cell and tissue destruction due to the capability of antibodies to activate the complement cascade. The complement cascade is important process by which the immune system destroys a target of the immune system.

UVA1 phototherapy provides combinations of UVA1 phototherapeutic effects including, but not limited to, decreasing activated T-lymphocytes, improving the function of macrophages, and overall modulating the immune system to improve the ratio of cellular populations. Wherein said UVA1 phototherapeutic effects indirectly decreases the adverse effects of dysfunctional immune cells working against the body, and decreases collateral damage to bystander cells and/or tissue. Wherein said UVA1 phototherapy causes the dysfunctional immune cells to be down-regulated in several ways, including but not limited to, a decrease in populations of immune cells that are auto-reactive against the body, a decrease in available antigens for the immune system to react against, and an increase in protective features. Wherein said decrease in populations of immune cells that are auto-reactive against the body result in less cells damaging “self' targets throughout the body. Wherein said decrease in available antigens for the immune system to react against is due to the improved clearing capabilities of macrophages due to the application of UVA1 light. Wherein said increase in protective features including, but not limited to, the TH2 concentration. Wherein said dysfunctional immune cells are immune cells including, but not limited to, dysfunctional T-lymphocytes attacking self

The direct effects of UVA1 phototherapy are combinations of one or more effects including, but not limited to, a decrease in aforementioned harmful populations of T-lymphocytes, an increase in aforementioned protective populations of T-lymphocytes, improved capabilities of macrophages to clear cellular debris, and improved signaling capabilities of macrophages. Wherein said decrease in harmful populations of T-lymphocytes is a result of a combination of one or more process including, but not limited to, induction of apoptosis by UVA1 light. Wherein said increase in protective populations of T-lymphocytes is a result of a combination of one or more process including, but not limited to, the interaction of UVA1 light at the cellular level, the interaction of UVA1 light at the sub-cellular level the interaction of UVA1 light with blood, the interaction of UVA1 light with tissues, and the interaction of UVA1 light with other fluids. Wherein said improved capabilities of macrophages to clear cellular debris as a result of a combination of one or more processes including, but not limited to, tissue destruction by external causes, sunburn, other environmental exposures, internal causes, apoptosis, and cell death caused by malignancies. Wherein said improved signaling capabilities of macrophages is a result of a combination of one or more process including, but not limited to, the interaction of UVA1 light at the cellular, the interaction of UVA1 light at the sub-cellular, and the interaction of UVA1 light at the extracellular environment level.

The indirect effects of UVA1 phototherapy are combinations of one or more effects including, but not limited to, a decrease in release of inflammatory cytokines and other chemicals, a decrease in the creation of autoantibodies and immune complexes, a decrease in inflammation, and a decrease rate of generation of cell and tissue damage. Wherein said decrease in release of inflammatory cytokines and other chemicals is a result of a decrease in populations of inflammatory and immune system cells activated against an antigen and/or self Wherein said decrease in the creation of autoantibodies and immune complexes is a result of decreased cellular debris and decreased time for debris to linger. The decreased linger time of cellular debris lowers the probability of a dysfunctional immune system formation of autoantibodies and a dysfunctional immune system response against components of the “self”. An example of said dysfunctional immune system is an immune system of an SLE patient. Wherein said decrease in inflammation and generation of cell and tissue damage as decreased immune complex deposition results in processes including, but not limited to, a decreased formation of reactive oxidant species against immune system targets, a decreased activation of the compliment cascade, and decreased reactions as a result of decreased immune-complex formation and deposition.

Preferred embodiments of the present invention provide the additional effects of UVA1 phototherapy to combinations of one or more selected body sites including, but not limited to, internal sites, external sites, and whole body irradiation. Whereby the additional effects of UVA1 phototherapy are combinations of one or more effects including, but not limited to, a decrease in risk or risk factors for a specific disease, a decrease in risk or risk factors for a specific illness, a decrease in the development of a specific disease, a decrease in the likelihood in the development of a specific disease, a decrease in the development of a specific condition, a decrease in the likelihood in the development of a specific condition, a prevention of the development of a specific disease, a prevention of the development of a specific condition, a decrease in the progression of an already acquired illness, a decrease in the progression of an already acquired disease, a decrease in the progression of an already developed illness, and a decrease in the progression of an already developed disease.

Wherein said decrease in risk or risk factors for a specific illness and/or diseases includes, but is not limited to, target disease, and malignancies such as breast cancer.

Wherein said decrease in the development of or decrease in the likelihood to development a specific disease and/or conditions includes, but is not limited to, a target disease.

Wherein said prevention of the development of a specific disease and/or condition includes, but is not limited to, prophylactic treatment of target disease, prophylactic treatment of transplant rejection, and prophylactic treatment of GVHD.

Wherein the phototherapy methods provided by the preferred embodiments of the present invention effect a decrease in the progression of an already acquired or developed illness and/or disease including, but not limited to, said target disease, Multiple Sclerosis, Rheumatoid Arthritis, and GVHD.

Additional UVA1 Phototherapy Physiologic Effects

UVA1 phototherapy effects a physiologic response in said target diseases through mechanisms including, but not limited to, interactions with said target cells, said photo-protection mechanism, molecular interactions, atomic interactions, and atomic electron interactions. Wherein said atomic interaction changes the energy of orbital electrons resulting in a change in the probability of combinations of one or chemical reactions involved with physiologic processes. The patient's physiologic systems affected by UVA1 phototherapy are combinations of one or more systems including, but not limited to, innate immune system. The patient's physiologic processes affected by UVA1 phototherapy are combinations of one or more processes including, but not limited to, apoptosis, necrosis, autoimmunity, singlet oxygen processes, heme oxygenase processes, cell processes stimulation, cell processes down-regulation, and oxidative burst. The patient's body cell types affected by UVA1 phototherapy are combinations of one or more cell types including, but not limited to, macrophages, and T-cell types. The patient's physiologic materials affected by UVA1 phototherapy are combinations of one or more materials including, but not limited to, complement, proteins, secretions, whole cells, partial cells, whole tissues, and partial tissues.

Apoptosis

The useful benefit of the intra-cellular process known as apoptosis is the elimination of undesirable cells including, but not limited to, infected cells, exhausted cells, mutated cells, undesired cells, and damaged cells. The products of apoptosis are combinations of one or more apoptotic products including, but not limited to, apoptotic blebs, and apoptotic bodies. Following apoptosis, said products of apoptosis are normally retained within membranes as apoptotic blebs. Said products of apoptosis undergo eventual clearance by macrophages via the process of phagocytosis. Phagocytosis occurs when a phagocytic cell recognizes the presence of specific surface markers on apoptotic blebs. Said surface markers on apoptotic blebs are combinations of one or more surface markers including, but not limited to, phosphotidyl serine, antibodies, and other chemical and/or protein markers. Said surface markers on apoptotic blebs are subsequently recognized by one or more receptor molecules on phagocytic cells. Said phagocytic cell types are cell types including, but not limited to, macrophages. Said phagocytic cells are cells capable of biological functions including, but not limited to, phagocytosis.

The apoptosis process is impaired in combinations of one or more of said target diseases due to combinations of one or more apoptosis impairments including, but not limited to, an energy-dependent impairment of apoptosis, a receptor dependent impairment, and a surface marker dependent impairment. The prior art has attempted to address said apoptosis impairment, with limited success, using apoptosis modulating medications that have various degrees of drug induced responses including, but not limited to, increase apoptosis in order to make-up for said apoptosis impairments. Said apoptosis modulating medications are combinations of one or more drugs, including, but not limited to, Azathioprine, cyclophosphamide, rituximab, monoclonal antibodies, hydrochloroquine. Said apoptosis modulating medications promote undesirable toxic side effects. Said apoptosis modulating medications are chosen from drug categories including, but not limited to, corticosteroids and/or quinolones. In the human body, energy processing is dependent on adenosine triphosphate (“ATP”). SLE-like target diseases are diseases including, but not limited to, said target diseases. Published research has shown that the ATP deficit in said SLE-like target diseases is a result of mitochondrial dysfunction (Perl et al., 2004). In general, apoptosis is dependent on ATP to function properly. A failure of the apoptosis process usually results in an increase in the generally undesirable alternative cell-death process of necrosis.

The two types of apoptosis are spontaneous apoptosis, and activation-induced apoptosis. An ATP deficit increases spontaneous apoptosis and inhibits activation-induced apoptosis. In said SLE-like target diseases, T-Lymphocytes can be ATP depleted and can have mitochondrial hyperpolarization, which results in increased spontaneous apoptosis and decreased activation-induced apoptosis of the T-cell. In addition, the ATP deficit in said SLE-like target diseases also makes apoptosis less likely to occur in the T-cells and/or other cells of said SLE-like target diseases and necrosis more likely to occur in said T-cells and other cells of said SLE-like target diseases (Perl et al, 2004).

Macrophages

A beneficial function of macrophages is the clearance of said products of apoptosis through the activation of the Fc receptor on the macrophage. The activation of the Fc receptor on the macrophage relies on the engagement of the macrophage Fc receptor by phospholipid epitopes on the surface of said products of apoptosis including, but not limited to, apoptotic blebs. In said SLE-like target diseases, multiple impairments are implicated including, but not limited to, impaired apoptosis, and impaired clearance of apoptotic bodies by macrophages and other phagocytic cells. The reduced phagocytic function of macrophages in said SLE-like target diseases was established and linked to impaired Fc receptors in two separate published studies (Koene H R, 1998, Herrmann M, 1998).

Said products of apoptosis have surface materials including, but not limited to, procoagulant phospholipids, such as cardiolipin. The presence of the phospholipids results in the production of anti-phospholipid antibodies, including, but not limited to, anti-cardiolipin antibodies. Anti-phospholipid antibodies normally neutralize procoagulant phospholipids and opsonize apoptotic bodies. Recently, an animal study was published demonstrating that apoptotic body immunization resulted in anti-phospholipid antibody formation which indicates that the surface markers of apoptotic blebs result in the activation of the autoantibody network involved in said SLE-like target diseases and may be involved in the pathogenesis of SLE and/or said SLE-like target diseases. (Cohen P L, 2002).

Necrosis

Necrosis is a form of cell and tissue breakdown. In general, necrosis is not ATP dependent. Necrosis can trigger the activation of dendritic cells and inflammatory cells. Necrosis results in the release of material including, but not limited to, proteases, oxidizing molecules, INF-α, chemotactic factors, and other inflammatory mediators in addition to cellular components usually contained in a cell membrane. The necrosis process can result in the release of autoantigens. The presence of said autoantigens can stimulate the activation of the immune system autoantibody network leading to said SLE-like target diseases. Necrosis is a normal physiologic process that normally occurs in response to a variety of factors when activated appropriately. Said material released from necrosis is also known as the products of necrosis. Said products of necrosis is a combination of one or more materials including, but not limited to, inflammatory mediators, autoantigens, and pathogens. Said products of necrosis get released into tissues and into the circulatory system. Transient necrosis typically occurs following processes and/or actions including, but not limited to, trauma. Transient necrosis generally results in the transient appearance of IgM and other antibodies to clear debris and damaged tissue.

In said SLE-like target diseases the necrosis process can be pathologic because of the failure of the apoptosis processes, which results in abnormally increased necrosis processes. The abnormal increase in necrosis processes can be due to impaired clearance of said products of apoptosis. Lingering products of apoptosis have an increased probability of conversion to products of necrosis as a result of the products of apoptosis entering the secondary necrotic state. Said secondary necrotic state occurs after apoptotic blebs linger and are not cleared (Gaipl et al, 2007). If the products of apoptosis are not cleared properly by phagocytosis then the probability of necrosis increases for the lingering products of apoptosis. The pathologic increase in necrosis processes causes an increased generation of products of necrosis. The increased amount of products of necrosis is a problem compounded by an immune system with ineffective debris clearance capabilities, which can lead to said SLE-like target diseases.

Autoantibodies

The formation of an autoimmune network of autoantigens and autoantibodies is a necessary and appropriate response to normal debris formation and occurs regularly in the normal human body. Said normal debris formation results from normal processes including, but not limited to, normal apoptosis, and normal necrosis.

In said SLE-like target diseases, the pathogenic process of autoimmunity develops as a result of excessive exposure to nuclear autoantigens. Autoantigens are released by secondary necrotic processes attributed to lingering apoptotic cells that have not been cleared. Lingering apoptotic cells can be cells that have begun apoptosis but not completed apoptosis. The lingering apoptotic cells eventually begin necrosis and/or result in blebs that are lingering. Secondary necrosis can result from the necrosis of lingering apoptotic cells and/or the necrosis of lingering blebs. The pathologic basis of autoimmunity in said SLE-like target diseases can be a result of a combination of one or more physiologic processes including, but not limited to, a failure in recognition and uptake of apoptotic and necrotic cells and/or an accumulation of secondary necrotic cells in secondary lymph organs (Gaipl et al, 2006).

Adenosine Triphosphate

The cells of a patient with combinations of one or more said SLE-like target diseases generally suffer from a deficit of adenosine triphosphate (“ATP”). Mitochondria are the energy-producing components of cells in the human body. Mitochondrial dysfunction can result in an ATP deficit within a cell. Mitochondrial dysfunction is indicated by increases in mitochondrial mass. Mitochondrial dysfunction has a probable partial relationship to the generation of membrane hyperpolarization. Mitochondria membrane potential controls activity of redox sensitive caspases. (Perl et al, 2004). Redox sensitive caspases are involved in the processes of apoptosis. Permanent mitochondrial dysfunction can result in impaired apoptosis and can, in part, be causative to macrophage dysfunction. Macrophage dysfunction can cause increased necrosis. Increased necrosis has been shown to lead to said SLE-like disease indications. The decrease in ATP is also implicated in chronic fatigue seen in patients with a combinations of one or more said SLE-like target diseases, which is assessed as aerobic insufficiency and measured by peak oxygen consumption.

Innate Immune System

The innate immune system can be impaired in patients with a combination of one or more said SLE-like target diseases. A patient with a combination of one or more said SLE-like target diseases can have an impaired immune system that suffers from decreases in the available materials used to effectively defend against infection. Said available materials are comprised of combinations of materials including, but not limited to, proteins, complement, MBL, CRP, SAP and DNAse-1. In addition, a patient with a combination of one or more said SLE-like target diseases can have a defective immune system that suffers from a diminished neutrophil oxidative burst. The overall result of a patient with a combination of one or more said SLE-like target diseases with immune system defects has resulting conditions including, but not limited to, increased risk of infection, a decreased clearance of cellular debris, and a secondary formation of autoantibodies. Said increased risk of infection has been shown to be the primary cause of mortality in a patient with a combination of one or more said SLE-like target diseases.

The oxidative burst is one of the most effective bactericidal mechanisms of the immune system. The oxidative burst results, in part, from the generation of free radicals by white blood cells and/or phagocytic cells. A particularly important free radical for effective oxidative burst is singlet oxygen. The oxidative burst is crucial for bacteria killing in a functioning immune system, and is involved in phagocytosis, chemotaxis, and cellular recruiting. Published data shows that singlet oxygen concentration is pathologically low in the oxidative burst of a patient with a combination of one or more said SLE-like target diseases.

Additional Pathologic Issues in Target Diseases

The T-Lymphocyte is implicated in said target disease processes. An ATP deficit has been shown to occur in macrophages and/or in T-cells of many patients with a combination of one or more said SLE-like target diseases. The ATP deficit in T-cells causes the T-cells to be resistant to apoptosis and/or become susceptible to necrosis.

Serum components are affected to various degrees in a patient with a combination of one or more said SLE-like target diseases. Said various degrees of affectedness range from mildly affected to significantly affected. The literature indicates that complement is suspected in the pathogenesis of said SLE-like target diseases. The role of complement has not been completely elicited. The early part of classical pathway of complement system activation has been shown to be part of the protection against development of said SLE-like target diseases. The activation of complement contributes to some of the tissue damage in said SLE-like target diseases. The first component in the complement system is C1q1, and C1q is necessary for effective opsonization. Opsonization works with soluble IgM to clear dying cells, As an example of opsonization process implications, a homozygous deficiency of C1q has been shown to be a powerful human susceptibility gene for the development of autoimmunity. A deficiency of complement results in impaired clearance processes including, but not limited to, impaired clearance of apoptotic cells, and impaired clearance of apoptotic cells following opsonization. More than half of patients reported having a homozygous deficiency of C1q have combinations of diseases including one or more said SLE-like target diseases.

In addition to said complement in the body, phagocyotosis promoting factors in serum have been shown to be decreased in many patients with a combination of one or more said SLE-like target diseases. Said phagocytosis promoting factors are combinations of materials including, but not limited to, complement, α2-HS glycoprotein, pentraxin CRP, and histidine-rich glycoprotein. (Gaipl et al, 2007, McGrath, 2007, Botto et al, 1998).

Function of UVA1 Phototherapy Interactions

Prior to the development of UVA1 phototherapy preliminary theories were proposed regarding various parameters in said SLE-like target diseases that had specific implications in the pathogenesis of the illness. The preliminary theories described implications including, but not limited to, autoantibodies are destructive, autoantigens are bystanders, autoimmunity drives said target diseases, ultraviolet light is harmful, oxidants are counterproductive, and antibodies either neutralize or opsonize. Refinements of, and advances beyond, said preliminary theories have developed as a result of extensive research in the application of UVA1 phototherapy devices and methods. The extensive UVA1 phototherapy research demonstrates that said SLE-like diseases have implications leading to theories including, but not limited to, autoantibodies are physiologic, autoantigens are pathogenic, excess debris drive the disease, ultraviolet-A1 light is beneficial, UVA-2 can be harmful, UVB can be harmful, UVC can be harmful, oxidants can be remedial, and antibodies kill.

Useful methods by which the UVA1 phototherapy affects combinations of one or more said SLE-like diseases pathogenesis are described herein.

Singlet Oxygen

UVA1 has the capability to generate singlet oxygen. Singlet oxygen, has capabilities including, but not limited to, promote apoptosis, trigger apoptosis, activate macrophage Fc receptors, conserve ATP, increases the concentration of available ATP, recharge leukocytes, deter necrosis, activate antibodies, and activate the gene for heme oxygenase (“HO-1”) production.

Apoptosis and UVA1 Phototherapy Interactions

UVA1 promotes apoptosis whereas UVB inhibits apoptosis. The promotion of apoptosis by UVA1 photons occurs through UVA1-induced singlet oxygen. Singlet oxygen promotes apoptosis by mechanisms including, but not limited to, acting as an oxidizer, breaking DNA single strands, depolarizing mitochondrial membranes releasing cytochrome-c, and via FAS and FAS-L induced mechanisms. Cytochrome-c is an endogenous stimulator of apoptosis (Godar D E, 2005). In addition, singlet oxygen activates JNK and p38 gene, prevents GF functioning, induces p53, inhibits kinases, induces T-Cell apoptosis, and preserves ATP which is essential for apoptosis. (Schieke S M, 2004 and Morita A, 1997). Kinases tend to inhibit apoptosis and therefore inhibiting kinases increase the probablity of apoptosis over necrosis.

Macrophages and UVA1 Phototherapy Interactions

UVA1 phototherapy enhances macrophage function primarily by the activation of the Fc receptor, improving the macrophage's capability of recognizing the macrophage's matching ligand on the target including, but not limited to, said products of apoptosis and/or other debris. The activation of the Fc receptor occurs due to singlet oxygen has been observed both in vitro and in vivo. The activation of the Fc receptor results in the enhancement of ingestion activity of the macrophage. The activation improvement is believed to occur by enhancement of Fc receptor mobility in the macrophage and/or phagocytic cell membrane by membrane peroxidation (by singlet oxygen). The peroxidation results in increased fluidity of macrophage membrane and the creation of cells with “normal” dying characteristics with increased mobility of phosphotidyl serine (“PS”) on the cell membranes.

By enhancing macrophage function a therapeutic benefit is achieved in said SLE-like diseases via the increased clearance of apoptotic bodies and/or other debris, and the reduced production of antiphospholipid antibodies. Wherein said antiphospholipid antibodies are combinations of one or more materials including, but not limited to, anti-Cardio Lipin antibody (“aCL”), anti-nuclear antibody (“aNA”), and anti-double stranded DNA antibody

ATP and UVA1 Phototherapy Interactions

UVA1 phototherapy also can mitigate the energy deficiency in patients with a combinations of one or more of said SLE-like target diseases since the resulting singlet oxygen an increase oxidative phosphorylation. Oxidative phosphorylation is the processes by which cellular energy sources such as ATP are generated. Oxidative phosphorylation occurs in response to UVA1 phototherapy when triplet state photosensitizers absorb UVA1 photon energy and transform it into molecular oxygen. The transfer of energy results in energy re-emitted and delivered at wavelengths including, but not limited to, 634 nm, for cytochrome oxidase absorption. Following the energy transfer and cytochrome oxidase absorption process the respiratory chain is stimulated and there is the comencing of electron transfer, which increases mitochondrial ATP production in cells, which has the indirect benefit of reducing fatigue in patients and promoting ATP to drive proper apoptosis in cells.

Low-dose UVA1 phototherapy delivers the proper effective dose of UVA1 to produce the required amount of singlet oxygen for desired effects. UVA1 phototherapy results in a combination of one or more effects including, but not limited to, increased ATP production enables improved apoptosis processes, enables and improves macrophage function, enables and improves innate immune function, accounts for a significant decrease in fatigue, and accounts for an overall decrease in disease activity. In patients with combinations of one or more said SLE-like target diseases the UVA1 phototherapy compensate and can correct for said SLE-like target disease related deficits. (Soussi B, 2004, 2005 and Landberg et al 2002).

Immune System and UVA1 Phototherapy Interactions

UVA1 has been demonstrated to specifically improve the oxidative burst in said SLE-like target diseases, said SLE-like target diseases have a generally impaired oxidative burst. UVA1 photons generate singlet oxygen within the neutrophil providing an improved oxidative burst. Additionally, UVA1 provides improvements in phagocytosis and chemotaxis allowing enhanced location and engulfment of targets and subsequent necessary destruction with oxidative burst. The immune system improvements resulting from UVA1 phototherapy results in an increased ability of patients with combinations of one or more of said SLE-like target diseases to fight off infection, breakdown debris, and clear debris. (Klotz, 2002)

Necrosis and UVA1 Phototherapy Interactions

UVA1 decreases the progression of cellular material to necrosis by increasing the concentration of singlet oxygen resulting in combinations of one or more beneficial effects including, but not limited to, the promotion of apoptosis, the activation of macrophage Fc receptor, the generation of ATP, and the recharging of leukocytes oxidative burst and ATP.

Antibodies and UVA1 Phototherapy Interactions

Autoantibodies can catalyze hydrogen peroxide using singlet oxygen within the fold of the antibody by using water and singlet oxygen resulting in the production of hydrogen peroxide and ozone. Singlet oxygen provided by polymorphonuclear cells (“PMNs”) promotes the generation of hydrogen peroxide when singlet oxygen reacts with an antibody that is attached to a lymphocyte.

UVA1 photons have the capability to improve the immune system and overall clearance of debris via increased hydrogen peroxide and ozone, and increases the immune system capability to fight infections.

UVA1 photons interact with tryptophan and/or other chromophores within the antibody, generating singlet oxygen. Cells with bound antibodies are found in tissues and in circulation and are therefore easily accessible to UVA1 photons through the skin and microcirculation. Adequate UVA1 accessibility to cells is beneficial since adequate UVA1 accessibility to cells results in increased formation of singlet oxygen by the antibody. The half-life of singlet oxygen is generally between 2-4 nanoseconds requiring the UVA-1 irradiated antibody to be pre-bound to cell surface for the singlet oxygen generating reaction to occur. In said SLE-like target diseases, there exists a prominence of pre-bound anti-lymphocyte antibodies, which increases the probability of the singlet oxygen generating reaction occurring. Hydrogen peroxide is mitogenic and therefore promotes cellular proliferation. Hydrogen peroxide promotes apoptosis, which results in the elimination of undesirable exhausted lymphocytes. (Wentworth et al, 2002, Reth, 2002 and Farber, 1994)

Overview of Singlet Oxygen Functions

Singlet oxygen, that can be generated by the application of UVA1 phototherapy has capabilities including, but not limited to, improved function of antibodies, enhanced T-Cell functions, increase cellular ATP, promote apoptosis over necrosis, activate macrophage Fc function, recharge the neutrophil oxidative burst, deter necrosis, and compensate for mitochondrial-based dysfunction.

Heme Oxygenase (“HO-1”) and UVA1

Not all benefits of UVA1 irradiation are a result of UVA1 induced oxidative responses. UVA1 generates singlet oxygen, which is oxidative, and this subsequently generates an oxidative response, such as HO-1 activation and/or the activation of antioxidant systems and repair mechanisms. The anti-oxidative response to UVA1-induced oxidative stress via singlet oxygen formation is an extremely significant benefit. Singlet oxygen is the most effective exogenous activator of the HO-1 producing gene known. The singlet oxygen required for the activation of HO-1 is provided by low-dose UVA1 phototherapy irradiation in a safe and effective manner.

Heme oxygenase is the rate-limiting enzyme of heme degradation. The heme degradation reaction is written as the following reaction:

Heme→CO+biliverdin (antioxidants)+Fe.

Subsequent to the heme degradation reaction the resulting Fe then goes on to stimulate Ferritin. Ferritin is an endogenous antioxidant and iron storage molecule.

In said SLE-like diseases UVA1 phototherapy promotes the generation of HO-1. HO-1 provides several important therapeutic functions referred to as HO-1 therapeutic functions. Said HO-1 therapeutic functions are combinations of one or more function including, but not limited to, down-regulation of mesangioproliferative glomerulonephritis, down-regulation of pulmonary hypertension, decreased coronary vasoconstriction, down-regulation of acute hypertension, down-regulation of pleurisy, down-regulation of interstitial fibrosis, down-regulation of atherosclerosis, and down-regulation of preeclampsia. Mesangioproliferative glomerulonephritis is one of the main causes of morbidity and mortality in patients with a combination of one or more said SLE-like target diseases.

UVA1 induction of the HO-1 gene may fend off longer term causes of morbidity and mortality in said SLE-like diseases, other diseases and normal patients, in addition to the immediate availability of said HO-1 therapeutic functions. A longer term benefit includes a decrease in avascular necrosis since singlet oxygen has anticoagulation properties (Lockshin Md., 2005). An additional longer term benefit of HO-1 includes a decrease in neurocognitive dysfunction. Said decrease in neurocognitive dysfunction is due to a documented improvement following UVA1 administration (McGrath H, 2005). A decrease in atherosclerosis due to HO-1 has anti-atherosclerotic properties (Wu B J, 2006).

The evidence supporting the role of HO-1 in UVA1 phototherapy therapeutic functions is well documented and published as evidenced by the following published research topics:
1. Hemin is a potent HO-1 inducer and therefore, the administration of hemin results is a reduction of proteinuria, amelioration of glomerular lesions, decreased immune depositions, and significantly decreased IgG anti-dsDNA in MRL/1 pr lupus mice (Takeda Y, 2004).
2. Mesangioproliferative glomerulonephritis developed in a patient with congenital deficiency of HO, as well as in HO-1 targeted mice.
3. Pleurisy responds to HO-1 (Willis et al, 1997).
4. Pulmonary inflammation and hypertension are prevented by targeted expression of HO-1 and improve with administration of CO.
5. One UVA1 treated patient with interstitial lung disease (“ILD”) showed ILD reversal paralleled by decreasing SLAM, with concomitant decrease then D/C of corticosteroids (McGrath).
6. Additional reports of decrease of dyspnea following UVA1 phototherapy (Polderman et al, 2004).

Additional important benefits from the induction of HO-1, include, but are not limited to, the amelioration of inflammatory bowel disease (“IBD”), amelioration of diabetes mellitus, amelioration of ceribrovascular accident (“CVA”), prevention of vascular stenosis, and provision of pregnancy benefits. Bilirubin and IL-10 are known to be effective treatments for IBD. UVA1 phototherapy promotes the generation of IL-10 and bilirubin. Re-stenosis occurs with combinations of said SLE-like target disease related coronary disease. Wherein the pregnancy benefits of UVA1 phototherapy include, but are not limited to, the capability of HO-1 to attenuate inflammatory cellular damage in placenta villous explants (Ahmed, 2000), evidence showing that HO-1 is decreased in preeclampsia (Lash et al, 2003), and evidence showing that recurrent miscarriages are associated with lower levels of placental HO-1, indicating that treatments capable of increasing the HO-1 induction will provide benefits (Zenclussen A C et al, 2005). Preferred embodiments of the present invention are capable of increasing HO-1 induction.

The preferred embodiments of the present invention make use of UVA1 LEDs to provide the following beneficial methods including, but not limited to, generate singlet oxygen, increase ATP production in cells, generate an overwhelming antioxidant response, activate the heme-oxygenase gene, promote vasodilatation, promote a decrease in vasoconstriction, promote decrease atherosclerosis, promote improved cellular respiration in ischemic and/or nutrient deprived cells, promote improved cardiovascular risk profile in diseased and/or normal patients, promote improved lung function of patients with respiratory illness from a primary or a secondary disease state, and promote improvements in diseases affecting cognitive function. Wherein said respiratory illness are combinations of one or more illnesses including, but not limited to, restrictive respiratory illness, and obstructive respiratory illness. Wherein said cognitive function diseases are combinations of diseases including, but not limited to, Alzheimer's Disease, Huntington's Disease, Parkinson's Disease, and Multiple Sclerosis. Wherein said improved cellular respiration improves conditions, including, but not limited to, a cerebrovascular accident (“CVA”), stroke, myocardial infarction (“MI”), heart attack, pulmonary embolism (“PE”), stenosis of blood vessels from atherosclerosis and vasoconstriction, thrombosis, transient ischemic attack, and embolic events.

### Cerebrovascular Accident

The goal of treating any brain trauma or injury is to decrease secondary damage following the precipitating event. The methods of use of the UVA1 LEDs incorporated in the preferred embodiments of the present invention perform functions including, but not limited to, CVA therapy functions. Wherein said CVA therapy functions are combinations of one or more functions, including, but not limited to, the use of UVA1 phototherapy to reduce the probability of an imminent stroke, reduce the severity of strokes in progress, improve recovery of neural tissue and brain tissue following a stroke, and improve patient outcome of CVA. The controlled application of UVA1 phototherapy from said UVA1 LEDs improves the recovery from a stroke, and reduces complications following a CVA. The activation of HO-1 and generation of singlet oxygen in a CVA improve the cellular respiration and generation of energy within affected and/or ischemic cells following the occlusion of vessels supplying said affected and/or ischemic cells with oxygen and nutrient rich blood. In addition, the anti-inflammatory nature of UVA1 phototherapy down-regulates the inflammatory process long-enough to allow physiologic processes including, but not limited to, salvage of cells not yet dead, prevention of cerebral edema, prevent bystander kill from necrosis of dead cells, and improve macrophage clearance of already dead and/or necrotic tissue. The phototherapy devices and methods of the preferred embodiments of the present invention are combinations of one or more methods including, but not limited to, whole body UVA1 phototherapy application, and direct-brain UVA1 phototherapy application.

Wherein said whole-body UVA1 phototherapy application is provided by the use of the methods of a combination of one or more preferred embodiments of the present invention upon the diagnosis of CVA. Subsequent to CVA diagnosis the patient is, for example, draped and/or wrapped in a UVA1 phototherapeutic blanket containing UVA1 LEDs with wavelengths known to generate singlet oxygen, activate HO-1, and produce beneficial effects of UVA-1 light. The UVA1 phototherapy is controllably applied from initiation of therapy, through the work-up in the emergency department (“ED”), during any subsequent procedures, and during recovery. The preferred embodiment for a whole body application of the present invention is a blanket form factor which is left on the patient for prescribed doses to allow for increased activation of HO-1, increased production of singlet oxygen and the provisioning of all of the benefits of the UVA1 phototherapy process. Wherein said subsequent procedures include combinations of one or more procedures including, but not limited to, catheterization, and craniotomy. An alternative form factor for the whole body UVA1 phototherapy application is one of the preferred embodiments of the present invention having a chamber form factor.

Wherein said direct-brain UVA1 phototherapy application provides the method implements following arrival of the patient to the ED, a patient typically undergoes initial medical procedures including, but not limited to, a computed topography (“CT”) scan and/or CT angiography and localization of the area of CVA. As an example, the physicians apply combinations of one or more applications of beneficial UVA1 phototherapy to the affected area including, but not limited to, catheterization, and brain irradiation. Wherein said catheterization UVA1 phototherapy application is a combination of one or more methods including, but not limited to, introducing one or more combinations of the preferred embodiments of the present invention following localization of the lesion, introducing a preferred embodiment of the present invention UVA1 phototherapeutic catheter with the beneficial UVA1 phototherapy emitter and a fiber-optic cable directed to the affected area of clot and/or areas of brain that previously were covered in blood and/or clot material. An additional method of the preferred embodiment of the present invention is for physicians to treat the clot in order to allow the beneficial UVA1 phototherapy through the previously occluded artery to access the affected tissue and/or ischemic and/or damaged cells. Wherein said brain UVA1 phototherapy application typically follows localization of the lesion and craniotomy, removal of overlying dura, where upon beneficial UVA1 phototherapy is applied directly to affected part of the brain to improve recovery of the cells and/or act as a cellular respiratory bypass if the clot had not already been resolved. Since UVA1 phototherapy increases ATP production in cells in addition to other beneficial effects of UVA1 phototherapy the UVA1 phototherapy is capable of improving cell recovery and/or act as a cellular respiratory bypass method. The application of the UVA1 phototherapy is timed in a manner that minimizes the error in delivering a phototherapy prescription. Similar use is made of the preferred embodiments of the present invention for stroke-like medical conditions including, but not limited to, head trauma with increased intracranial pressure and/or cerebral edema.

### Myocardial Infarction (“MI”)

Similar to the methods of the CVA UAV1 phototherapy, the application of UVA1 phototherapy to MI can be accomplished by a combination of one or more methods, including, but not limited to, whole-body, and directly to the affected tissue. Similar physiologic benefits are obtained in the MI UVA1 phototherapy as are derived in the CVA UVA1 phototherapy. The generation of singlet oxygen and the activation of HO-1 and other beneficial effects of UVA1 phototherapy result in benefits to a MI patient. The benefits derived by the phototherapeutic application of UVA1 to an MI patient is similar to those derived from a CVA patient, whereby the blood supply to tissue has been critically reduced and the tissue and/or cells become ischemic and are lacking oxygen, nutrients, and other necessary and beneficial components derived from the blood. Wherein said phototherapeutic procedures include combinations of one or more procedures including, but not limited to, cardiac catheterization, and direct-heart UVA1 phototherapy application.

Wherein said cardiac catheterization provides the phototherapeutic methods following an EKG diagnosis. An EKG has localization capabilities to determine the MI location. Typical MI locations include, but are not limited to, transmural infarct. Prior-art cardiac catheterization is typically used to open stenotic coronary arteries either as prophylaxis for angina or for various cardiac risk factors in a patient. Prior-art cardiac catheterization is also typically used following an acute MI to relieve the blockage and/or place stents to provide patency to the coronary vessels. Utilizing combinations of one or more the preferred embodiments of the present invention including, but not limited to, UVA1 emitters and fiberoptic fiber, a preferred embodiment of the present invention in a cardiac catheter form factor delivers phototherapeutic UVA1 phototherapy to the tissue downstream of the thrombus and/or embolus in the coronary vessels to improve the cellular metabolism and respiration immediately following an infarct. The phototherapeutic methods of the preferred embodiments of the present invention provide the benefit of increasing ATP and decreasing the inflammation of the area as in the CVA UVA1 phototherapy application to attempt to decrease the amount of dead or dying myocardial cells. The UVA1 phototherapeutic methods of the preferred embodiments of the present invention provide secondary benefits including, but not limited to, the decreased risk of a rupture of the wall of the heart where the infarct occurs, and a decrease in dead and dying cells with secondary benefit of maintaining better long term heart function profile. Said rupture of the wall typically occurs within the first week following an MI where the wall is weakened by tissue death and the lack of scar formation that has not occurred within the first week.

Wherein said direct-heart UVA1 phototherapy application provides the phototherapeutic methods following an EKG diagnosis and/or other diagnostic tool or diagnostic procedure and localization of the area of infarct. Thoracocentesis can also be used to place a combination of one or more preferred embodiments of the present invention in a catheter form factor to apply phototherapeutic UVA1 phototherapy within the pericardial sac to provide UVA1 phototherapy directly to the affected tissue on the surface of the heart. The direct-heart UVA1 phototherapy application methods provide benefit in an MI that was in the first two layer of the heart, and to a lesser degree provide benefit in a transmural infarct allowed by the tissue layer penetration capabilities of UVA1 phototherapy. The penetration capabilities are dependent of tissue thickness and composition and vary among patients and among sites on the same patient. A combination of direct-heart UVA1 phototherapy application and intracardiac UVA1 phototherapy application within the ventricles and/or atria to irradiate the internal layer of the heart in a transmural infarct. The intracardiac UVA1 phototherapy application is accomplished utilizing a preferred embodiment of the present invention in a cardiac catheter form factor. Wherein said cardiac form factor of also incorporates light sources including, but not limited to, UVA1 LEDs capable of proving UAV1 phototherapy effects while minimizing the error approximating a UVA1 phototherapy prescription. Whole body UVA1 phototherapy is beneficial following and/or during MI to reduce complications and increase rate of recovery.

### Pulmonary Embolism

Following the lodging of an embolus or formation of a thrombus in the lung, the blockage of blood to parts of the lung prevent the exchange of oxygen and carbon dioxide within the lungs as well as oxygen and nutrient rich blood to pulmonary tissue. The blockage can potentially cause death within a relatively short period of time if the blockage is a large-scale blockage of a high-flow vessel in the lung. Over a relatively longer period of time, a relatively smaller blockage can potentially cause death of part of the lung and serious complications and/or death. Combinations of one or more of the preferred embodiments of the present invention are used to treat PE in a similar manner as to treat MI and CVA. The methods to treat PE are categorized as whole-body and direct lung UVA1 phototherapy applications. Benefits are derived from use of the methods of the preferred embodiments of the present invention when applied to PE including, but not limited to, providing cells a bypass supply of additional energy until the primary problem is solved. Whole-body irradiation is beneficial by providing ATP to cells of the whole-body to help reduce the risk of combinations of one or more PE complications including, but not limited to, multi-system organ failure and/or shock and/or death of the patient. Said PE complications which can occur with any form or manifestation of a PE.

Wherein said whole-body irradiation used in PE is similar to the methods used in MI and CVA with the timing of the UVA1 phototherapy application controlled to suit the duration of a PE phototherapy. Wherein said direct-lung UVA1 phototherapy application has methods dependent on the thickness of the lung parenchyma, a bronchoscope with a combination of one or more preferred embodiments of the present invention is utilized to provide UVA1 phototherapy directly to the areas of the lung that have been infarcted either from lung airways and passage by access via mouth, pharynx, and trachea or by external application via mediastinoscopy or access via penetration through chest or thoracic wall, and placement of one or more combinations within the pleural sac.

### Subdural Hematoma

The methods used to provide the benefits of the preferred embodiments of the present invention to subdural hematoma (“SDH”) follow a similar pattern for the whole-body and/or direct tissue irradiation, similar to the MI, AVD and PE uses. Benefits are derived following the evacuation of blood from the surface of the brain to provide access to the exposed brain. The exposed brain is then irradiated with UVA1 phototherapy. Blood that sits on viable tissue can be toxic if the static blood persists long enough. Tissue can become ischemic following evacuation of blood, blood clots or other products of a subdural hematoma. Application of UVA1 phototherapy to the involved tissue provides combinations of one or more useful effects including, but not limited to, revitalization by increasing recovery of ischemic tissue, revitalization by increasing recovery of toxic tissue, and increasing energy production within affected and surrounding tissue. The methods provided by combinations of one or more preferred embodiments of the present invention provide said increasing energy production via combinations of or more effects of UVA1 phototherapy including, but not limited to, decrease brain tissue death, decrease subsequent permanent complication to the patient, decrease mass effect, and decrease compression of the brain. Wherein said mass effect and/or complication to the patient are a result due a pathological increased volume of blood in a closed environment of the cranium resulting in compression and ischemia of the brain tissue. Use is made of the preferred embodiments of the present invention to maximize the approximation of a phototherapy prescription capable of providing combinations of one or more tissue therapies including, but not limited to, tissue rescue, ischemic tissue rescue, tissue revitalization, ischemic tissue revitalization, tissue vasodilatation, and ischemic tissue rescue. Said preferred embodiments of the present invention incorporate means capable of providing combinations of one or more UVA1 phototherapeutic methods including, but not limited to, control of ATP generation. Wherein said control of ATP generation method promotes a combination of one or more useful phototherapeutic effects including, but not limited to, vasodilatation. Vasodilatation is promoted by combinations of one or more UVA1 phototherapy effects including, but not limited to, the activation of HO-1 from the UVA1 phototherapy. Ischemic tissue is rescued and revitalized as a result of the preferred embodiments of the present invention and provide the useful methods including, but not limited to, vasodilatation, and anti-inflammatory properties of UVA1 phototherapy to treat increased debris in the brain.

Additional use of the preferred embodiment of the present invention is made in conditions of re-perfusion injury that can occur in MI, PE, and/or CVA following revascularization of infarcted tissue. The infarcted tissue if treated with the UVA phototherapy prior, during and/or after the medical procedures to restore blood flow in order to reduce the re-perfusion injury that can occur.

### Burn Treatment

Burn levels are generally classified as degrees including, but not limited to, first degree, second degree, and third degree. Burns of varying levels have varying levels of inflammation, infection and blood flow within the affected region and surrounding tissue. UVA1 phototherapy provides beneficial effects including, but not limited to, decreasing inflammation of the body, decreasing inflammation of one or more burn sites, and increasing immune system activity to fight the increased chance of infection resulting from the burns. In a manner similar to MI, AVD, SDH, and PE, methods of use of the preferred embodiments of the present invention include, but are not limited to, whole-body UVA1 phototherapy application, and direct-tissue UVA1 phototherapy application. Said whole-body UVA1 phototherapy application is applied following the burn and/or throughout the course of the patients recovery to increase preservation of tissue, improve tissue recovery, improve repair capabilities, improve healing capabilities, improve granulation capabilities, and increase recovery time. Said direct-tissue UVA1 phototherapy application provides benefits by direct application of UVA1 phototherapy to the affected tissue. Direct-tissue UVA1 phototherapy application results in benefits including, but not limited to, rescue of ischemic tissue, rescue of nearly dead tissue, improved skin, cell and tissue healing processes, improvement of the immune system capabilities to fight off infection, a decrease in the pain and inflammation through a combination of one or more mechanisms including, but not limited to, increasing blood flow to the area, and increasing energy for cellular repair. Decreasing harmful aspects of the inflammation rx, and decreasing anti-inflammatory and pain generating cytokines and secretions in the blood and surrounding tissue. The positive effects of inflammation includes the removal of damaged tissue, removal of infection or other foreign bodies at a given site, it also includes the repair of damaged tissue or the rebuilding of a wounded area to reconstitute the bodies skin barrier as well as tissue and fascial planes within the body following injury and/or infection. The negative effects of inflammation include fibrosis and/or scar formation, also the inflammatory response of inflammatory cells like lymphocytes, macrophages, neutrophils also damages tissues in the process of trying to repair damage, which results in pain by nerve activation, irritation, heat and swelling by vasodilatation of blood vessels, etc. and if it becomes chronically inflamed, such as in SLE, said SLE-like diseases, arthritis. If the inflammatory cascade does not halt and damage occurs from continuous activation of inflammatory cells and the release of their cellular contents. In some instances fibrosis can actually be positive if an area was made weak by damage/injury and now is filled in with scar, such as an incision line, although scar tissue is generally undesirable from a patient's cosmetic preferences.

### Wound Healing and Grafting

The use of the present invention for wound healing and grafting UVA1 phototherapy application follows the principles of UVA1 phototherapy benefits and has similarities with the MI, AVD, PE, SDH, and burns. Some wounds heal better with increased ATP in cells to promote increased wound healing and a decrease in inflammation and resulting pain. The use of the preferred embodiments of the present invention provides an increase in available energy, a mild oxidant reaction in affected tissue, and an activation of HO-1. The UVA1 phototherapy benefits are combinations of one or more benefits including, but not limited to, improves healing capabilities, improves tissue granulation, a decrease in recovery time, and an increase in immune system protection from infection following tissue grafting. The use of the preferred embodiments of the present invention provides combination of one or more secondary benefits including, but not limited to, a decrease in evulsions of wounds, a decrease in wound dehiscence, a decrease in local infection, a decrease in graft rejection, and a decrease in systemic infection. The use of the preferred embodiments of the present invention also improves recovery of wounds in situations where the wound needs to remain open to heal including, but not limited to, infected wound sites, abscesses, open surgical fields, and fasciotomies. The use of the preferred embodiments of the present invention also improves healing of exposed muscle and other surrounding tissue. Wherein said surrounding tissue is in one or more dynamic tissue states including, but not limited to, fully involved, partially involved, not involved but at risk of involvement, not involved and at no risk of involvement, and slightly injured.

The use of the preferred embodiments of the present invention for grafting including, but not limited to, cutaneous epidermal graft, split-thickness graft, free-flap graft, muscle graft, tendon graft, and complex grafts. Wherein said complex grafts include combinations of one or more grafts including, but not limited to, whole-organ grafts, partial organ graft, and tissue graft. Wherein said tissue graft is a combination of one or more tissue grafts including, but not limited to, kidneys tissue graft, liver tissue graft, and heart tissue graft. Wherein said partial organ graft is a combination of one or more partial organs including, but not limited to, partial liver graft. Wherein said whole-organ graft can be organs including, but not limited to, kidney, heart, and liver. The use of the preferred embodiments of the present invention applied directly to the harvest site before removal of graft and/or to the graft site before the graft is placed, results in useful benefits for wound healing, including, but not limited to, the healing of wound edges, improved tissue granulation, and an increased success of the “taking” of the graft to the graft site.

### Graft-Failure

Typically graft failure and/or transplant failure results from a lack of blood supply and/or oxygen to generate energy to the cells of the graft. Graft failure can result in tissue death, host rejection of graft, and necessitates removal of the graft. The graft failure mode categories includes graft failure before graft harvest, graft failure before graft placement, and graft failure after graft placement. Said graft failure can be acute, sub-acute, and/or chromic rejection.

The use of the preferred embodiments of the present invention is beneficial for reducing said graft failure attributed to said before graft harvest graft failure mode. The use of the preferred embodiments of the present invention before harvesting of graft tissue increases the cellular respiration of the tissue prior to grafting and increase graft survival following excision.

The use of the preferred embodiments of the present invention is beneficial for reducing said graft failure attributed to said before graft placement graft failure mode. Use of the preferred embodiments of the present invention is made on the receiving site before the graft is placed on the location requiring said graft, in order to promote graft maintenance prior to graft transplantation, such as while graft is in transit or following graft harvest and placement of graft on sterile table and/or container for use during same operation.

The use of the preferred embodiments of the present invention is beneficial for reducing said graft failure attributed to said following graft placement at site graft failure mode. Use of the preferred embodiments of the present invention is made when graft is in place to increase viability of the cells while the wound “takes” to the graft site resulting in a decrease in graft failure and an improvement in graft healing at wound site. Grafts are comprised of one or more combinations of graft types including, but not limited to, graft from self to self, graft from donor to self, and grafts from manufactured sources. Wherein said graft from self to self type has graft sub-types including, but not limited to, muscle grafts, and free-flap grafts. Wherein said graft from donor to self has graft sources including, but not limited to, human donor, and animal donor. Wherein said human donor may be from a live donor graft, or a cadaver donor graft. A graft can be used as-is, processed, stored, and reconstituted.

### Wound Healing Secondary Effects

Due to the beneficial effects of UVA1 phototherapy on wound healing and graft transplantation, there is decrease in combinations of one or more secondary healing effects including, but not limited to, inflammation, improved healing capabilities, improved clearance of debris from wounds, and improved remodeling of the wound site following healing, a decreased incidence of scar formation, and a decreased incidence of contraction of the wound which results in a poor cosmetic result and decreased function of tissue. Wherein said remodeling of the wound site is comprised of combinations one or more materials including, but not limited to, tissue, collagen, elastin and fibrin. Wherein said poor cosmetic result is a combination of results including, but not limited to, keloid scars, and contractures. The decreased incidence of scar formation and/or contracture results due to a prevention of an overactive healing and/or remodeling process at the wound site, decreased inflammation during would healing, and an improvement in blood flow to the wound. Said wound site is a combination of one or more wound types including, but not limited to, simple wound, and complex wound. Wherein said improvement in blood flow to the wound provides useful benefits including, but not limited to, increased delivery of nutrients to the wound, and increased delivery of beneficial repair cells to the wound.

A wound is generally described as a cutaneous skin wound. Wherein said cutaneous skin wounds can exist at any combination of the various layers of the skin, and/or at all layers of the skin. Wherein said wounds take a dynamic combination of one or more wound forms including, but not limited to, a epidermal wound, a wound through the epidermis and dermis, a wound through all layers of the skin into muscle and other tissue, a wound within a cavity of the body or in one or more organs or collections or tissues or any combination thereof

The whole process requires monitoring and feedback to optimize the UVA1 phototherapy prescriptions. Typically the material and the processes needed to heal are available within the body but the immune system and the tissue healing processes are generally not making effective use of the material available in pathological processes. The material needed for the beneficial chemical reactions and biological change is generally present within the patient tissue, but the reaction is either not initiated, is stalled at some point in the chemical reaction process, is not functioning properly, is reacting at an abnormally high rate, or is reacting at an abnormally low rate. If material needed is not available then this can be introduced via dietary changes, chemical intake, and drug intake. The UVA1 phototherapy is capable of providing the energy of activation to activate the chemical processes improving the use of the material present to effect a biological change resulting in a therapeutic benefit. As an example, use is made of a antigens that the body has known defenses against to create the conditions where the T-cells have the proper antigen to create the singlet oxygen and turn on the heme-oxygenase gene to generate a desirable response.

### Sequencing Wavelengths

The benefit of sequencing wavelengths is the overall reduction of total amount of UV required to obtain benefits by delivering a photobiological optimized sequence of light capable of promoting the desired biologic effect and eliminating harmful and/or otherwise random and/or unoptimized sequences of UVA1 and/or light. Delivering the specific sequence of UV wavelengths promotes the associated specific desirable photobiological reactions. The best choice is to avoid sequences that promote harmful processes and avoid sequences that have a neutral effects.

Except for prior art phototherapies that make use a single discrete wavelength the prior art phototherapies emit multiple wavelengths at the same time. The contemporaneous wavelengths are comprised of more than one wavelength range and/or wavelengths at the same time. This is in contrast to sequenced wavelengths which emit a first spectral irradiance and one or more additional wavelengths in an optimized pattern. Said optimized pattern is comprised of temporal, spectral and spatial changes which are the active phototherapy sequences providing the benefits of phototherapy. The advantage of the present invention is to avoid the sequences that are harmful or useless, and to provide the sequences that are the most beneficial.

### Methods of use Common to Treatments Discussed Herein

Use is made of the preferred embodiments of the present invention to maximize the approximation of a phototherapy prescription capable of providing combinations of one or more tissue therapies including, but not limited to, tissue rescue, ischemic tissue rescue, tissue revitalization, ischemic tissue revitalization, tissue vasodilatation, and ischemic tissue rescue. Said preferred embodiments of the present invention incorporate means capable of providing combinations of one or more UVA1 phototherapeutic methods including, but not limited to, control of ATP generation. Wherein said control of ATP generation method promotes a combination of one or more useful phototherapeutic effects including, but not limited to, vasodilatation. Vasodilatation is promoted by combinations of one or more UVA1 phototherapy effects including, but not limited to, the activation of HO-1 from the UVA1 phototherapy. Ischemic tissue is rescued and revitalized as a result of the preferred embodiments of the present invention and provide the useful methods including, but not limited to, vasodilatation, and anti-inflammatory properties of UVA1 phototherapy to treat increased debris in the brain.

### Examples

### Phototherapy Capsule and Method for Gastrointestinal Tract.

A useful method of the preferred embodiments of the present invention is the therapeutic application of UVA1 light from at least one of a capsule to the cells in proximity to the walls of the gastrointestinal tract in order to modulate the ratio of lymphocyte cell populations in proximity to the walls of the gastrointestinal tract. Said capsule is of a size that can be swallowed, and vary in size dependencies on potential size and oral capabilities.

The preferred embodiments of the present invention incorporate any suitable means capable of emitting phototherapeutic light including, but not limited to, the preferred LED types. Wherein said preferred LED types include, but is not limited to, AlN based LED type, GaN based LED type, InGaN based LED type, and AlInGaN based types.

A first preferred embodiment of the present invention makes use of one or more phototherapeutic capsules introduced into the gastrointestinal tract by introducing and making use of phototherapy methods including, but not limited to, capsule(s) swallowed or otherwise inserted into the gastrointestinal tract by an insertion means. Wherein said inserting means including, but not limited to, arthroscopic surgery. Wherein said phototherapeutic capsule contains one or more phototherapeutic capsule components including, but not limited to, a battery, a UVA1-LED, an activation triggering mechanism means, a computer chip to store data, a central processor unit, (“CPU”), any suitable communications means, and a pH monitor. The preferred embodiments of the present invention, once entering the activated state, will deliver phototherapeutic prescription including, but not limited to, UVA1 light, to the lumen of the bowel for the treatment of specific gastrointestinal tract diseases, including, but not limited to said target diseases, Crohn's disease, and ulcerative colitis. Wherein said activated state is controlled by an activation trigger means. Wherein said activation trigger means is comprised of a combination of one or more trigger means including, but not limited to, a trigger communications means, and a trigger sensor means. The device incorporates a phototherapy state machine means, and can enter or exit the activated state via said activation trigger means.

Said phototherapeutic capsule will incorporate self-test means. The various preferred embodiments of the present invention will incorporate any suitable means capable of providing useful methods including, but not limited to, self-test methods to provide the useful method of increasing the accuracy of optimization of the phototherapy prescription.

Said phototherapeutic capsule will optionally be coated with a specific enteric coating that is responsive to a specific pH within the lumen of the bowel. The gastrointestinal tract, which includes the bowel, has a pH gradient that starts in the mouth and varies through the gastrointestinal tract to the large bowel and finally the rectum, the capsule will be coated with a specific material that will dissolve when it encounters a specific pH that is correlated to the location of the bowel desired. Upon dissolution, the trigger state changes, the capsule is activated and the capsule phototherapeutic LED is engaged to deliver light to the interior of the bowel, large intestine or small intestine, depending on what type of coating is on the capsule. Said capsule incorporates any suitable means capable of providing the useful methods of phototherapeutic flux including, but not limited to, UVA-LED, UVA1-LED, and UVA1C-LED, UVB, UVC, visible, and infra-red.

The preferred embodiments of the present invention optionally incorporates any suitable magnetic coupling means capsule components that allow the capsule to be manipulated inside the body. Wherein said magnetic coupling means capsule components are responsive to a combination of one or more magnets external to the phototherapeutic capsule including, but not limited to, external magnets rotating around the body in a controlled manner, magnets in an external hand-held device, magnets within a fabric or blanket draped over the patient, magnets in a garment designed to be worn by the patient with an internal power source or with an external power-source, and magnets in nearby phototherapy capsules in the proximity of the phototherapy capsule. Wherein said magnetic means is comprised of combination of one or more suitable means including, but not limited to, electro-magnetic, and permanent magnetic coupling means. Wherein said magnetic coupling capsule components are comprised of materials including, but not limited to, iron, nickel, cobalt, rare-earth magnetic materials, samarium-cobalt, and neodymium. Wherein said magnetic coupling capsule components are combinations of magnetic flux emitters and active electromagnet and/or passive magnetically responsive materials. Said magnetic coupling means including, but not limited to, mutual magnetic resonance power transference means to remotely supply energy to power said preferred embodiments of the present invention. The preferred embodiments of the present invention optionally incorporate a loop energy capture means. Said loop energy capture means is comprised of any suitable means capable of collecting electromagnetic radiation including, but not limited to, a loop created by multiple segments of capsule connections. Said loop energy capture means includes capabilities of receiving control signal included with the power signal capture.

The magnetized component means allows the swallowed capsule(s) to be rotated and/or translocated in a dynamically controlled pattern in order to deliver light to the prescribed tissue in the gastrointestinal tract. The magnetized component means allows the swallowed capsule(s) to be rotated and/or translocated in a dynamically controlled pattern in order to keep the capsule phototherapy effect on at least one tissue surface of the lumen for optimized delivery of a phototherapeutic method. Wherein said dynamically controlled patterns are combinations of one or more patterns including, but not limited to, adaptive phototherapy pattern. Wherein said parts of the gastrointestinal tract are preferably the combinations of one or more prescribed phototherapy target diseases tissue including, but not limited to, said target diseases, Crohn's Disease tissue, and Ulcerative Colitis tissue.

The preferred embodiments of the present invention relates to a phototherapy capsule capable of being orally ingested, travel along the gastrointestinal tract, providing a phototherapy effect, and preferably excreted in the stool or otherwise removed. Capsules include primary power sources, and power converting means to provide power to the light emitting components, preferably LEDs selected to provide a sequence of photons to closely match a prescribed phototherapy. An example of said prescribed phototherapy is the treatment of gastrointestinal tract diseases, including, but not limited to, said target diseases, Crohn's disease, and ulcerative colitis, using a gastrointestinal disease phototherapy prescription. Wherein said gastrointestinal disease phototherapy prescription is a combination of one or more wavelengths including, but not limited to, UVA1, and UVA1C, administered in a gastrointestinal disease directed application to gastrointestinal disease tissue, of a dynamic flux sequence.

### Background

Prior art capsules as described by Imran et al., in U.S. Pat. No. 7,160,258 B2 dated Jan. 9, 2007 included herein by reference in it's entirety. The capsules described by Imran have a variety of attributes that allow for diagnosis methods. However a limitation of the prior art is the inability of the capsule to actively position itself, and the inability to position itself relative to other capsules within the vicinity, the inability to spatially couple with other capsules in the same vicinity, and an inability to provide the specific wavelengths capable of treating diseases responding to UVA1 light including, but not limited to, Lupus, Crohn's disease, and ulcerative colitis.

The preferred embodiments of the present invention overcomes the limitation of the prior art, including, but not limited to, Imran's invention, by incorporating controllable alignment means, wherein said controllable alignment means is comprised of the following alignment means including, but not limited to, magnets, movable magnets, permanent magnets, suction pumps, suctions ports, electromagnets, gears, active friction surface, electrostatic, hook and loop fasteners. Wherein said alignment means is responsive to an alignment control means, to substantially align one or more capsules in the gastrointestinal tract. Said capsules have optimization controls to align relative to gastrointestinal tract and to other capsules.

Each capsule has a data communication and data storage means to process location data to determine with a high degree of accuracy the relative position of other capsules and body position, through determining means including, but not limited to, vibration means, radio frequency identification (“RFID”) means, and electromagnetic identification means.

Wherein said alignment means programmatically adapts to changing environmental conditions external to the capsule conditions for individual capsules individually, or as a group of capsules, to maintain the lowest energy expenditures.

Said preferred embodiments of the present invention include any suitable means capable of powering active components. Said power source means is preferably comprised of combinations of one or more of the following power source components, including, but not limited to, primary batteries, remotely coupled resonant electromagnetic energy transfer means, rechargeable batteries, catalyst, piezoelectric material, fuel cells, bio-generator, functioning cellular components modified to power a light source. Wherein said piezoelectric material is arranged to provide one or more combinations of functions including, but not limited to, convert electric energy to mechanical energy, and convert mechanical energy to electrical energy. Wherein said mechanical energy is comprised of one or more combinations of mechanical energy forms including, but not limited to, sound, and ultrasound. Said piezoelectric means is any suitable means capable of providing the useful method of promoting the clearance of opaque particles and liquids between the capsules and the gastrointestinal wall for a phototherapy.

Said bio-generator is comprised of modified cellular and sub-cellular elements or components of surrounding blood, blood serum, and fluid. Said sub-cellular components include combinations of one or more components scavenged from tissue including, but not limited to, mitochondria elements in electrical connection to a charge accumulator means. Wherein said charge accumulator means is comprised of electrical components including, but not limited to, a rectifier element and a capacitance element, conductors, a frequency generating means, alternating current (“AC”) to direct current (“DC”) converter means, DC to DC power converting means, DC to AC power converting means, and power control circuits. Wherein said modified cellular elements are preferentially scavenged from host or alternatively introduced from an external source. Said bio-generator incorporates any suitable means capable of providing the useful methods including, but not limited to detecting a failed component, and replacing a failed component with a functioning component. The mitochondria used is preferably from the host, either obtained previously or during the phototherapy session. Mitochondria are optionally used from non-host source(s) in which case the preferred mitochondria have a compatible DNA signature as the host.

A second preferred embodiment of said present invention is reusable.

A third preferred embodiment of the present invention is disposable.

A fourth preferred embodiment of the present invention is partly recyclable and partly disposable. Said fourth embodiment of the present invention incorporates combination of one or more battery components including, but not limited to, rechargeable batteries, and disposable batteries.

A fifth preferred embodiment of the present invention is partly consumable, releasing substances in non-toxic concentrations including, but not limited to, carbon dioxide and water. Said fifth embodiment has fuel delivery means and combinations of one or more fuel processing means and combinations of one or more fuel harvesting means.

The preferred embodiments of the present invention comprise combinations of one or more energy transference means between the coupled chains of capsules. The set of capsules is ingested within a time range that allows sets of the capsules to line up in a variety of arrangements including, but not limited to, a linked chain, whereby the capsules travel along the gastrointestinal tract. Capsules can have zero to many connections to other capsules depending on the purpose. Coordinated combinations of capsules require specific numbers of connections to accomplish phototherapy goals, including, but not limited to, one connection for end of chain purpose, two connections for linked one dimensional pattern, teo connections for a ring pattern, three or more connections for three dimensional pattern. Wherein the capsules link with two or less connections form a logical one-dimensional form, or a ring if there are no end points with only a single connection. Wherein said capsules with two links form a ring to be complete. Said capsules with three links form a local two-dimensional plane that can be attached to a three dimensional array. Wherein links with four or more connections create a logical three-dimensional array. Said connections can change state changing the logical structure of the capsule array.

The capsules systematically determine, in near real time, the current optimal connections and adjust connections to adjacent capsule, by utilizing internal rotational motion, releasing certain connections and/or making new connections. A train of capsules can be created dynamically as the capsules enter the stomach and head toward the bowel. Said connections are combinations of one or more connection types, including but not limited to, tethered connections, hook and loop, and magnetic.

A combination of one or more capsule types can be ingested, wherein each capsule type has a different combination of one or more function. The first capsules swallowed are typically of type path-maker capsules, thereby making a path through the gastrointestinal tract. Said path-maker capsules will establish a path for the other capsules types in the chain. Said path-maker type capsules have combinations of one or more capsule properties including, but not limited to, extra fuel for moving through the gastrointestinal material.

The capsules are distinguishable to medical personnel and patients by graphics including, but not limited to, alphanumeric markings and by digital identification means with a serial number and capability ROM. Wherein said capability ROM including, but not limited to, the type of capsules and the capsule capabilities. The capsule type and the capsule capabilities, is useful in operating scenarios, and in test scenarios, when the optimization control program is determining if a capsule swap is worth the cost in energy and/or probability of increasing the phototherapy effectiveness. The exact order of individual capsules of the same type does not change the effectiveness of capsules of the same type as they are in communication with each other and also in communication with capsules of differing types. A mesh network communication means is utilized to increase reliability. Said mesh network communication means is comprised of any suitable means for creating and adapting to low power communications, with the master controller, and the backup controller. Each controller is capable of switching roles in a distributed network according to an algorithm that increases the effectiveness of the phototherapy. Each previous capsule transmits and receives information including, but not limited to, analysis results, model results, and sensor data, the measurements and other findings of the on-board sensors, comprised of combinations of one or more components including, but not limited to, proximity to targeted tissue, video, optical, pH, temperature, strain, and chemical composition of biological agents. Said capsule form factor of the preferred embodiments of the present invention has the ability to share sensor data between capsules. The information from the first capsule is shared to the rest of the capsule chain as needed. Likewise information from other capsules is shared as needed between capsules. For example, the information from the other capsules are also shared in a similar fashion as the first capsule except that the first capsule is unique, since the first capsule does not have a lead capsule to receive information from. However, multiple capsules make use of information from previous phototherapy capsule sessions taking into account that the information from previous phototherapy sessions capsule is older information and not as useful in determining current conditions. To reduce system requirements including, but not limited to, memory, communications bandwidth, and design requirements, the bowel is mathematically modeled using mathematical techniques including, but not limited to, finite element analysis, and perturbation theory, whereby the raw information obtained by the local on-board sensors are compressed and reduced to best-fit model parameters prior to communication to neighboring capsule. Resulting parameters are transmitted with preference to compressed data to conserve memory and bandwidth. Data is transmitted as needed on a requested basis, and in a broadcast manner. In order to conserve power and to increase accuracy of treatment, the therapy is applied at the calculated optimal time.

The data from the previous sessions is generally older and not all of the information is relevant except for seeding the adaptive predictive control algorithms. Said capsules optionally incorporate one or more microcomputer unit (“MCU”). Said MCU is capabilities include, but is not limited to, operating under a real time operating system.

In addition said capsule chain has distributed CPU and communication network modules running independent algorithm threads handling local events and data collection, and in addition multitasking CPU, multiple CPU, and networking threads.

In addition said capsule chain collection has an optional mechanical tethered connection one or more nearby capsules. Alternatively, the capsules are programmed to release said mechanically tethered connections to mechanically arrange the capsules into programmed shapes useful for improving the effectiveness of said phototherapy prescription.

Both capsules in a connection, in any combination, can release a tether connection if the optimization requirement arises, or the tether connection can be transferred from one capsule to another.

Said phototherapy capsules are ingested and/or surgically inserted via catheter, after ingesting optional prescription preparations to prepare the gastrointestinal tract for the therapy. Said tethered connection may have more than one connection per tether provided the tether has more than one point of contact.

Said phototherapy capsules are capable of morphing shapes by programmatically changing connections including, but not limited to, magnetic connections, permanent magnetic connections, electromagnetic connections and tethered connection. Capsules are organized dynamically into topologies that act as a system, wherein certain capsule surface are presented to the environment to accomplish a useful goal including, but not limited to, controlling the position of the chain relative to the gastrointestinal tract tissue. Using controlled friction flow, and another side(s) that works against the flow utilizing a high friction surface and has the capability to coordinate either low or high resistive surfaces in order to increase the probability of moving towards gastrointestinal phototherapy target tissue surface and away from flowing gastrointestinal material. The useful method of coordinating movement using the surface of the chain allows for increased effectiveness in the therapeutic delivery system. When the therapeutic system delivers a phototherapy, then a close proximity of the phototherapy light source to the gastrointestinal phototherapy target tissue increases the effectiveness of the phototherapy because of the reduction of flowing gastrointestinal material that either partially filters or is opaque to the phototherapy light source.

Said preferred embodiments of the present invention incorporates combinations of one or more heat transfer means including, but not limited to, a conductive heat transfer means, a convection heat transfer means, and a material flow heat transfer means. Said capsule embodiment of the present invention incorporates a material transfer heat transfer means capable of input flow of gastrointestinal material at one temperature, capable of heat transfer from capsule to gastrointestinal material, and capable of output flow of the gastrointestinal material.

Electrically conductive connections between the capsules provide the useful method to transfer electrical energy from a capsule that has electrical power to a capsule that has the opportunity to provide phototherapy flux because of the position relative to the phototherapy prescription. Electrical connectors can be dynamically created, as needed, using switching elements within the phototherapeutic capsules. Said tethered connections incorporate any suitable means capable of providing the useful methods including, but not limited to, providing mechanical connections to other capsule(s), providing electrical connections to other capsule(s), and the tether communication capabilities. Wherein said tether communication means is comprised of any suitable means including, but not limited to mechanical vibrations means, communication means, electrical communications means, and optical communications means.

Said capsules have fiber optic components to distribute phototherapeutic light to the tissue or to other capsules for distribution to tissue.

The capsule chain delivers electrical energy to the capsules in the chain that are within range of the targeted tissue, thereby providing a substantially static location for phototherapy as the capsules moved past a target tissue area, as the capsules move past the target tissue the capsule(s) in range of the target tissue will receive power from internal capsule sources and from attached capsules. It is possible that some capsules will not be in range of the target tissue and will therefore only serve the useful method of providing capsule power to the capsules within range. Said capsule power comprises combinations of power forms including, but not limited to, electrical, mechanical, nuclear, chemical, optical, and hydraulic.

A sixth embodiment of the present invention includes internal magnets of the capsule are also capable of transferring mechanical energy to the other capsules. The capsule shell is capable of moving relative to the internal magnets. Internal motors and gears system are used to create movement of the capsule surfaces used in combination with screws and mechanical fasteners. The capsule shell is comprised of any suitable translucent material including, but not limited to, acrylic, and quartz. Wherein said translucent material has a shape that allows for increased ease of swallowing and re-location to the phototherapeutic site. Wherein said capsule shell has any suitable means capable of providing the useful method of allowing the capsule to maintain a position relative to the prescribed phototherapy site. The capsule further incorporates any suitable means capable of providing the useful purpose of providing light in the direction of the prescribed phototherapy site, including, but not limited to, mechanical rotation means in a spherical internal chamber, an array of LEDs that are array addressable. Wherein said array of LEDs is comprised of combinations of one or more array topologies including, but not limited to, a convex array, a concave array, and a flat array. The preferred topology is the convex array, and the preferred method of using the convex array is to power only the LEDs that have an effective path to the prescribed phototherapy site. The methods to control power to the LEDs that have an effective path to the prescribed phototherapy site is provided by any suitable means including, but not limited to, a phototherapy site detection means. Wherein said phototherapy site detection means incorporates any suitable means capable of providing the method of analyzing the effectiveness of each LED in said array of LEDs.

A seventh embodiment of the present invention incorporates a tether system creating mechanical forces, including, but not limited to, tension, torsion, and strain. The capsule chain can morph into additional shapes and therefore can change direction of motion and move relative to the gastrointestinal material flow, in order to maintain prescribed positions in the targeted therapy site. Said prescribed phototherapy site target changes dynamically to best fit a phototherapy prescription. Capsule movements relative to gastrointestinal fluid flow includes any suitable movement including, but not limited to, forward movement, oscillatory movement, rotation movement, reverse movement, and lateral movement.

The capsule size varies dependent on capsule type. A filler-type capsule is generally smaller than the therapeutic capsule type. The filler-type capsule does not have an active phototherapeutic effect, but is useful in creating shapes that allow increased effectiveness of the therapeutic capsule type(s). Filler-type capsules are useful solutions to mechanical gastrointestinal dysfunction due to diseases including, but not limited to, diverticulitis. A filler-type capsule incorporates any suitable means with capabilities to provide the useful methods including but not limited to increasing the efficiency of the phototherapy, reflect phototherapeutic light to the prescribed phototherapy target tissue, and to redirect light through a fiber optic cable. A balloon type capsule type includes an inflatable balloon means. Said balloon-type is re-inflatable and reusable and capable of storing gas, liquid, or plasticized solids. Said gas is comprised of gaseous compounds including, but not limited to, methane. Said gas has gas sources originating from gas sources including, but not limited to, bacteria in the bowel and may be used to power the device via any suitable means including, but not limited to, a fuel cell. Wherein said inflatable balloon means have the capability of inflating with gas, liquid, or other substances in a dynamic fashion to provide the useful method of changing shape in a dynamic manner, and to increase mass for methods of manipulating the positioning of one capsule to another blocking a capsule. Wherein said balloon capsule types can provide a simulated locomotion to assist in the undulations of the gastrointestinal tract.

Any one or more capsules in a mesh network make use of the internal control algorithm that allows the capsules to match as close as possible a phototherapy prescription.

The topology of the capsule chain varies dynamically and incorporates topologies including, but not limited to, mesh network topology, a linear topology, a three-dimensional topologies, and a ring in the gastrointestinal tract. Wherein the LEDs are arranged in combinations of one or more logical shapes including, but not limited to, the outside, and the inside of the ring. The control algorithm maintains records of the logical topologies of the capsules may also represents the physical topologies of the capsule formation, but most likely any one of the logical shapes will have a topology other than representing the physical shape, whereby the logical topologies represent critical functions including, but not limited to, current electrical power connections, predictive solutions, and available resources.

Fuel-types capsule are optimized using minimal energy to re-supply the chained capsule formation with supplemental energy during extended phototherapy sessions. The fuel-type capsules identify their presence using capsule communications means. The capture-type capsules are used in an expanding tether configuration to communicate using capsule communications means with capsules including, but not limited to, retrieve the fuel-type capsule types and conversely release spent fuel-type capsule types making room for additional fresh fuel-type capsules. The fuel capsules optionally move into the internal capsule multi-link chain mesh network. Fuel-type capsules will optionally be swallowed on a staggered and delayed time basis to provide continuous power supply over an extended period.

The internal and external surfaces of the linked chain have different capabilities and are therefore optimally positioned with respect to other function types of capsules. To create the optimal form and predict the optimal changes in the mesh structure and communicate these predictive solutions to other capsules. Optimal tracking algorithms are used to minimize energy use, and minimize loss of material to reduce spent fuel cells as a goal. The incorporated software system includes any suitable means capable of providing the methods of a real time operating system capability. Additional communications will be made to external transceiver to indicate the additional fuel schedule required to patient and/or to medical personnel.

The majority of capsule types will incorporate fuel, wherein the useful advantages of the fuel-type capsules are the re-supply of fuel to a phototherapy site. Fuel-type capsules may have additional capabilities, including, but not limited to, capture capabilities to connect to a mesh network, and to capture other free floating capsules.

The preferred embodiments of the present invention show the useful method of shaped capsule assemblies of dynamic topologies are formed as a linked chain set, under dynamic manipulation and control. The fuel-type capsule types have uses including, but not limited to, being retained after the primary method to supply power has been completed and are then retained to build structure as needed or optionally jettisoned as needed, or optionally rearranged. A fuel-type capsule will have an energy source selected from one or more of the following energy sources including, but not limited to, batteries, acid consuming batteries, fuel cells, mechanical energy to electrical energy transfer. Said acid consuming batteries will optionally obtain acid from the material flow in the gastrointestinal tract. Fuel conversion means including, but not limited to, a heat engine, and a fuel cell. Wherein said fuel cell makes use of methane and other hydrocarbon based chemicals in the gastrointestinal tract to convert from hydrocarbon to water and carbon dioxide and electrical power.

The capsules on the surface of the linked chain set have multiple modes of operation including, but not limited to, free spin. Said free spin mode of operation reduces the friction on the adjacent material flow. Another mode is to twist out chlorofluorocarbon slippery Teflon-like surfaces, a second mode is of operation presenting a sticky surface, suction and propulsion, and a third mode is a mechanical attaching mechanism.

The linked chain shape has the capability to continuously and operationally move up relative the gastrointestinal fluid flow and maintain a position in the prescribed gastrointestinal position. The movement from the optimal position is minimized by actively assisting the gastrointestinal fluid flow and/or the material flow to be diverted by any suitable active flow assisting means incorporated into the preferred embodiments of the present invention including, but not limited to, the phototherapy capsule preferred embodiments of the present invention.

Another useful goal of the present invention is to maintain the multi-mode multi-therapy type capsule close to the treatment area and in direct contact with the tissue.

If a capsule senses that it is failing, or is commanded by using the capsule communication means in the decision tree of a hierarchical capsule group comprised of components including, but not limited to, a neighboring capsules and remote capsules that it is to move in a direction by changing combination of one or more connection, including, but not limited to, tethered and magnetic coupling connections. Any suitable method of changing capsule connections allows the phototherapy type capsules to move toward the therapy region within the internal connections of the assembly.

Said capsule communication means has capabilities including, but not limited to, a messaging system. Wherein said messaging system is comprised of components including, but not limited to, a time stamp, a machine readable system clock, private key public key cryptography system, memory, data input means, data output means. Said preferred embodiments of the present invention authenticates the messages with a public key corresponding with the private key the message was electronically signed with. The capsules build a hierarchical model of the mesh network and are able to determine which capsules are designated to send commands to the other capsules. All communication links are authenticated and verified to determine the validity of the message. Logical groups of capsules are created to perform specific goals. Each logical group has a hierarchical structure that allows each logical group to effectively communicate, assign actions, and complete tasks as a logical group. Each logical group is registered with the governing group to provide inter-group effectiveness for completing task requiring more than one logical group. Each capsule belongs to one or more combinations of logical groups including, but not limited to, local, regional, and global logical groups. Commands and requests are issued between the capsules and the logical groups to make an effective control system to complete the overall task assigned to the capsules including, but not limited to, the optimization of the phototherapy prescription. Said messaging system incorporates any suitable means to provide methods of command execution for commands including, but not limited to, start command, stop command, pause command, move command, twist command, connect command, join group command, exit group command, read input command, write output command, re-start command, self-test command, test group command, send message command, validate message command, and execute message command.

Said preferred embodiments of the present invention are comprised of one or more components including, but not limited to, a master controller. Wherein said master controller incorporates suitable means to collect data, analyze data, output an optimized control signal, and to communicate with capsules to manage overall task of said logical groups to provide an optimization to the phototherapy prescription. The useful purpose of said master controller is to have a single point of control at any given time for any given task. The useful purpose of said master controller is also to perform command conflict resolution and to eliminate potential conflicting commands. Wherein said independent decisions are made and decision results are stored locally and are communicated to other capsules including, but not limited to, the master controller. The capsules create a mesh network communication means. Some internal controls are made independently of other capsule controls in order to reduce the unnecessary communications that are not useful except within a single capsule. In addition some internal capsule decisions are independent of the master capsule and thus only pertinent results are communicated to the master capsule. The master controller is preferably a capsule that has established a master communication link channel with the external phototherapy controller. The master controller is a role that a capsule can take on or acquire the rights to the master controller role by transferring roles using an acknowledgment method. The master controller role is unique in that the master can generate the unique authentication tokens needed for the communications role, the action role, the transferred master controller provider, and the transferred master controller recipient role.

The master controller role is controlled by an master controller token. Said master controller token is comprised of one or more components including, but not limited to, electronic data, physical identification means. Said master controller token can be passed using acknowledgment based communication means. The token will have a fail safe means that will not allow for a situation where there is no master controller present including, but not limited to, a watchdog timer, and a keep-a-live signal. Any capsule that has the master controller capability that has not been under the control of the master controller for a critical control period of time may initiate a master controller role initialization sequence. Said critical control period of time is defined as the period of time to continue normal operations and depends on the goals of the normal operations. An uncontrolled capsule may initiate a master controller role if there is no other master controller in communication with said uncontrolled capsule. In a control scenario where there is a conflict between two potential master controllers, then an agreed upon resolution mode of operation will be entered, whereby the multiple master controllers will follow a suitable controller dispute resolution method to determine and assign the master controller token. Said dispute resolution method will include weighted combinations of one or more methods including, but not limited to, original controller, most recent controller, capability comparison, reliability comparison, and random selection. Wherein said capability comparison provides an end result of determining which capsule is the most effective capsule to be granted the master controller token. Wherein said reliability comparison provides an end result of determining which capsule is the most effective capsule to be granted the master controller token. Wherein said original controller comparison provides an end result of determining which capsule is the most effective capsule to be granted the master controller token. Wherein said most recent controller comparison provides an end result of determining which capsule is the most effective capsule to be granted the master controller token. Wherein said weighted combinations is any suitable method including, but not limited to, the method of selecting the most recent controller with the highest reliability and the highest capabilities, or the original controller if the results are equivalent to within an acceptable error of observation.

Logical capsule sub-groups belong to logical capsule groups and take roles according to any suitable rules based optimization means and methods of use. Each logical group will behave according to the expected and agreed upon roles prior to executing a command based on a decision. The rules and the decision logic and techniques will be selected according to the requirements of the decisions being considered by the group. For example, suitable decision logic is used for deciding which capsule with external communication capabilities will handle the connection to the external master controller. The decision will be based on the reliability of the communications links to the external master controller and the reliability of the connections to the capsules included in the logical group, capsules can elect to observe the decision process in that logical groups communication channel and obey commands if the commands are valid. The most basic rule that all other rules are subject to is the rule that the capsule are to provide the optimal services to the patient. A suitable operating and programming language for the capsule control system is any suitable methods including, but not limited to, the Java programming language, and assembler language.

Said fluid flow has material compositions including, but not limited to, gastrointestinal fluid flow, and blood vessel fluid flow. Wherein said gastrointestinal fluid flow is comprised of combinations of one or more compositions including, but not limited to, fecal matter, and bile. Wherein said material compositions has combinations of one or more characteristics including, but not limited to, static composition, and dynamic compositions.

Channels of capsule and/or material flow within the dynamically linked chain assembly are created by organized flow of capsules and configuration of capsule surfaces including, but not limited to, sticky surfaces, medicated surfaces, suction surfaces, valve surfaces, light emitting surfaces, and smooth surfaces.

The capsules incorporate tagging means stored as analog or digital media within the capsule to indicate that it is within the patient of interest, and not within the vicinity of another patient. Said tagging means provides the useful method of identification allowing for the capsules to not be falsely controlled by an second patient's capsules control mechanisms, thus providing isolation of operation. Conversely, there are reasons to control capsules in a concerted manner between patients, as in the case of releasing a therapeutic effect simultaneously, in the case of an external event common to both patients, such as the room temperature fluctuating, or other safety precautions including, but not limited to, a fire alarm, a pandemic, and a regional disaster. Wherein said tagging means incorporates capabilities including, but not limited to, identification.

The preferred embodiments of the present invention incorporates any suitable means to provide useful methods including, but not limited to, an internal holding tank for material, an external holding tank for material, a suction means to provide capsule translation and rotation, material clearing capabilities, a stable gentle negative pressure suction connection to the gastrointestinal wall at the phototherapy site.

### Alternative Embodiment

### Catheter

An eight alternative preferred embodiment of the present invention has form factors including, but not limited to, a colonoscope, and a catheter. The alternative embodiment of the present invention incorporates an array of therapeutic light sources around the exterior, or interior, of the colonoscope, or catheter. Wherein said array of therapeutic light sources is comprised on one or more combinations of light sources including but not limited to, UVA-LED, UVA1-LED, and UVA1C-LED. Said array of therapeutic light sources incorporates any suitable means that provide the method of selectively and powering each light source independently in an addressable manner. Wherein interior LEDs require a translucent shell to allow the light to be delivered to the treatment location. Said translucent shell has characteristics including, but not limited to, substantial opacity to harmful wavelengths, and substantial translucency for phototherapeutic wavelengths. Wherein said opacity to harmful wavelengths may be partial opacity or full opacity. Generally, opacity to harmful wavelengths will be partial but the opacity is optimized to be effective reducing the hazard of harmful wavelengths below an acceptable and approved amount suitable to meet the requirements for a phototherapy prescription. Said translucent shell optionally includes one or more components including, but not limited to, spectral light filters, and phosphors converted, optically active compounds, photonic conversion compounds, and spectral filter solutions. The eighth alternative embodiment of the present invention is inserted through body orifices including, but not limited to, the rectum. The directed application of therapeutic light sources provides a useful and beneficial therapeutic light to one or more body components including, but not limited to, the interior of the bowel, bowel tissue and cells, lymph tissue, bacteria, and capillaries contained within the tissue. The various versions of the catheter form factor of the present invention are fashioned for use in specific body components including, but not limited to, an oral cavity, a uterus, and a vaginal cavity. Wherein said vaginal body component including, but not limited to, provide access to ovaries and abdominal cavity via the fallopian tubes, under eyelids, in ears, in nose, in throat, and arthroscopy. Wherein said therapeutic light sources include, but are not limited to therapeutic LED types. Wherein said therapeutic LED types are comprised of LED types including, but not limited to, UV, UVA, UVA1, and UVA1C. Wherein said therapeutic light has a dynamic spectral irradiance including wavelength ranges and discrete wavelengths including, but not limited to, EMR, UV, UVC, UVB, UVA, UVA1, and UVA1C, visible, and infrared (“IR”).

The eighth alternative embodiment of the present invention improves on the prior art by providing phototherapeutic prescription in addition to illuminating light for diagnostic methods. Prior art devices have been disclosed which provide method of providing light for illumination in a diagnostic procedure. Prior art device have been disclosed which provide for a surgical procedure including, but not limited to, tissue ablation. The prior art devices do not provide therapeutic light in combinations of phototherapeutic ranges including, but not limited to, UVA, UVA1, and UVA1C.

Said eighth alternative embodiment of the present invention includes a form factor that allows the UVA1 phototherapy to access the urethra and the organs, tissues, and spaces though the urinary tract to the kidneys.

### Alternative Embodiment

### Evacuation Device

The ninth alternative embodiment of the present invention is specialized version of the catheter form factor of the preferred embodiments of the present invention incorporates any suitable means capable of providing suitable methods evacuation methods that allows the ninth alternative embodiment of the present invention to transfer gastrointestinal material including, but not limited to, fecal matter down at least one internal channel of the tube. The useful method of withdrawing bowel contents from the lumen allowing improved method of delivering a phototherapy prescription of light to the walls of the bowel. The ninth alternative embodiment of the present invention will optionally incorporate a pressurized stream of a therapeutic liquid to break-up fecal matter and other bowel contents ahead of the advancing tip of the ninth alternative embodiment of the present invention. Wherein said ninth embodiment of the present invention incorporates any suitable means capable of providing the useful method to utilize an incorporated suction system to draw the mixture of solution and fecal material through an opening that runs through the interior of the catheter into an external waste holding chamber means. Wherein said external waste holding chamber means incorporates components including, but not limited to, disposable flexible bags, inlet port seals, waste treatment chemicals. Wherein said waste treatment chemicals are comprised of combinations of one or more compounds including, but not limited to, bleach, whereby the array of catheter LEDs that are incorporated in the ninth alternative embodiment of the present invention behind the tip of the catheter to have improved contact with the walls of the bowel providing the useful method of providing an improved surface area upon which to provide phototherapy prescription including, but not limited to, UV, UVA, UVA1, UVA1C. The preferred embodiments of the present invention incorporate any suitable means capable of providing the useful methods of said therapeutic liquid including, but not limited to, increasing the optical efficiency of the phototherapy. The increase in optical efficiency of the phototherapy is achieved by the removal of light filtering material or other opaque material from the gastrointestinal tract tissues in proximity and between the light source and the target tissue. Said array of catheter LEDs is comprised of one or more phototherapeutic LED type. Said phototherapeutic LED type is selected from one or more LED type to closely match the phototherapy prescription according to a suitable means capable of selecting the LED types. Said array of catheter LEDs is arranged in combinations of one or more topologies including, but not limited to, axial, radial, concentric, screw thread, variable, pseudo-random, random, and flexible. Said catheter LEDs are optionally protected by a translucent shroud means. Wherein said translucent shroud means has characteristics including, but not limited to, flexible, translucent, recyclable, washable, disposable. Wherein translucent characteristic is meant to be over combinations of useful spectrum including, but not limited to, phototherapeutic spectrum, and diagnostic illumination spectrum.

Wherein said therapeutic liquid is comprised of liquids including, but not limited to, saline solutions, caustic solutions, acidic solutions, purified water, buffered water, hydrogen peroxide solutions, oils, emulsifier, soaps, detergents, phosphate solutions, absorbic acid solutions, medicated liquids, psoralen solutions, casting material, plastics, catalyst solution, and biologically active agents. Said therapeutic liquid concentration varies over the phototherapy session including, but not limited to, transitions between two or more liquids used in combination, and transitions between two or more liquids used sequentially. Said preferred embodiments of the present invention is comprised of components including, but not limited to, an evacuation catheter means.

The width and length of the catheter preferred embodiments of the present invention will be manipulated by using a series of flow-stop valves within the catheter to fill the same therapeutic liquid used to break-up fecal matter to inflate various compartments along the catheter to more correctly fit the lumen of bowel in which said catheter with evacuation means resides. Said catheter with evacuation means is comprised of a components including, but not limited to, a valve power means, a control conduit means, and communication means.

Normally, the width of the catheter will vary over the length of the catheter. The length and the width of the catheter will vary as prescribed by the physician administering the phototherapy.

### Alternative Embodiment

### Nanoparticle Treatment Method

A tenth alternative embodiment of the present invention is comprised of a combination of one or more of manufactured nanoparticles, including, but not limited to, carbon nanotubes, spherical structures, optically pumped nanocrystal, a nanocrystal, and a helical nanoparticle structure. Wherein said optically pumped nanocrystals absorbs one or more photons and emits one or more photons of a differing more useful wavelength providing a photon wavelength conversion process. An example of said photon wavelength conversion method is the absorption of UVB photons and the subsequent resultant UVA1 light emission.

The outside of said manufactured nanoparticle will contain various binding domains for a combination of one or more desired target including, but not limited to, a molecule, fragment of a molecule, protein, tissue, targeted parasites, and malignant cell. Wherein said targeted parasites include, but are not limited to bacteria, viruses, parasites, and fungi. The preferred embodiments of the present invention is comprised of components including, but not limited to, specific nanoparticles that are systematically inserted into the body, including, but not limited to, local injection by needle, intravenous injection or otherwise. The invention further incorporates a combination of one or more binding domains including, but not limited to, a target domain, an elimination domain, an activity domain, and other domain.

Wherein said target binding domain means allows the particle to interact with one or more specific cell component types including, but not limited to, a tissue, a protein, a malignant cell. Wherein said target binding domain means allows the particle to bind to a specific region of a pathogen including, but not limited to, a virus, a parasite, a bacteria, and a fungi.

Wherein said elimination domain means allows the therapeutic nanoparticle to bind a target including, but not limited to, a component of the body, a compound in the body, and a cell in the body. A therapeutic nanoparticle elimination domain with one or more bound targets is referred to as a target elimination domain complex. When one or more, but not all, elimination domains on a therapeutic nanoparticle have been bound to targets the nanoparticle complex is referred to as a partially reacted therapeutic nanoparticle complex. When all the elimination domains on a therapeutic nanoparticle have been bound the therapeutic nanoparticle the therapeutic nanoparticle complex is referred to as a fully reacted therapeutic nanoparticle complex. When a minimum number of therapeutic nanoparticle domains have been bound to targets that meet the required number to be excreted, the therapeutic nanoparticle complex is referred to as an excretion-ready therapeutic nanoparticle complex. An excretion-ready therapeutic nanoparticle complex is excreted by any suitable method including, but not limited to, combinations of one or more methods used to excrete bile salts, acids, albumin, other chemicals, nitrogen, and proteins that get excreted by normal renal processes. The elimination domain means would optionally be hidden from the immune system until said therapeutic nanoparticle target binding means interaction was complete and subsequently the elimination domain means would be exposed to the immune system using combinations of one or more methods including, but not limited to, EMR, photodynamic therapy, phototherapy, optical detection, fluorescent tagging, a chemical, and other suitable means.

Wherein said activity domain means is the region that allows the particle to bind materials, cellular organelles, proteins or enzymes or any other components not listed within the body, cell, fluid or to be used in the resident environment of the activity domain for combinations of one or more useful methods including, but not limited to, the phototherapy prescription, and the further construction of the device in the body.

Wherein said other domain to target or bind the particle to components or substances of interest including, but not limited to, a foreign antigen domain that activates the immune system to attack cells including, but not limited to, HIV infected cells, thereby providing the preferred embodiments of the present invention with at least one capability to target a cell that is known to be infected with HIV and then eliminate the cell and virus without directly targeting the virus, but instead indirectly targeting the virus via an indirect targeting means. Wherein said indirect targeting means comprises any suitable means capable of providing methods including, but not limited to, target a cell that is known to be infected with HIV and then eliminate the cell and virus without specifically targeting the virus. An alternative embodiment of the preferred embodiments of the present invention would optionally directly target the virus.

Inside the body or the body component the nanoparticle performs useful methods including, but not limited to, alerting domain means allows the tenth alternate embodiment of the present invention to control an immune system alert signal means. Said immune system alert signal means provides combinations of one or more capabilities including, but not limited to, alerting the immune system to the location of specific pathogens including, but not limited to, viruses, and HIV. Said immune system alert signal means provides capabilities that allows the immune system to recognize typically hidden infections and then clear infections that otherwise would not be visible to the immune system. Wherein typically hidden infections include, but are not limited to, the process that occurs in HIV/AIDS whereby the HIV/AIDS infection hides in lymph node cells and is not visible to the immune system.

Inside the body or the body component the nanoparticle performs useful methods including, but not limited to, cell modifying domain means allows the tenth alternate embodiment of the present invention to control a cell modification means. Wherein said cell modification means provides combinations of one or more capabilities including, but not limited to, kill the cell, trigger apoptosis, and cell function modification. Said cell-modifying domain means allows the combination of one or more useful methods including, but not limited to, modification to cellular component. Said cell modifying domain means interacts with combinations of one or more cells including, but not limited to, a cell infected with a virus, a cell infected with a hidden virus, a defective cell, a cancerous cell, and any other cell that would otherwise not be eliminated but needs to be eliminated in order to improve the condition of the treated tissue and patient. Said cell modifying domain means interacts with combinations of one or more sub-cellular components including, but not limited to, dysfunctional cell organelles, dysfunctional mitochondria, and any other sub-cellular component that would otherwise not be eliminated but needs to be eliminated in order to improve the condition of the treated tissue and patient. Wherein said treated tissue is a combination of one or more tissues including, but not limited to, graft tissue, host tissue, biologically active cells.

Inside the body or the body component the nanoparticle performs useful methods including, but not limited to, alert synergistic nanoparticles of different functions of the presence of a component or the location of a virus so that the secondary nanoparticle can perform combinations of one or more functions.

Inside the body or the body component the nanoparticle performs useful methods including, but not limited to, immune alerting nanoscale particle, also known as an immune alerting nanoparticle, will optionally contain specific chemicals or signals to trigger combinations of one or more actions including, but not limited to, cell lysis, cell apoptosis, and mechanisms that allow the immune system to recognize a virus within the cell that would otherwise go undetected. Any suitable means are incorporated into said immune alerting nanoparticle that provide the useful methods including, but not limited to, alerting the immune system to an infected cell, promoting the halting of the replication of hidden viruses, and promoting the elimination of cancerous cells that are not effectively attacked by the immune system without the presence alerting nanoparticle. The markers on the immune alerting nanoparticle indicate the presence of a certain cell type, thus communicating the present position of an infected cell to the immune system. The immune alerting nanoparticle is useful for both hidden infections and acceleration of non-hidden infection immune response. The immune alerting nanoparticle incorporates any suitable means that allows the provisioning of combinations of one or more methods including, but not limited to, a method to assist an immune system component to locate a pathological target of a complementary type, and a method to assist an antigen to be discovered by an antibody of the same complementary type, a method to assist an immune system antibody to locate a pathological target of a differing type, and a method to assist an antigen to be discovered by an antibody of the differing type. Wherein said differing type is preferably a type for which the immune system has the ability to recognize through a native immune response or from a previously adapted immune response. Wherein said previously adapted immune response includes combinations of one or more immune responses including, but not limited to, a vaccine, or a common cold virus. Wherein said vaccine including, but not limited to, rabies vaccine, and yellow fever vaccine, and small pox vaccine. The preferred vaccine is chosen based upon previously determined efficacy data and the closest match with the least side effects for the condition to be treated. Said hidden infections display characteristics including, but not limited to, an antigen not detected by the immune system, and a retro-virus not being detected by the immune system. Said immune alerting nanoparticle incorporates any suitable means to provide the useful method of providing a translation of identification to the immune system one or more components of one or more hidden infections by immune system recognizable antigen signature tagging matter including, but not limited to, a cell with a hidden infection, a component of a cell with a hidden infection, a hidden virus, and a hidden compound. The immune alerting nanoparticle assists the immune system recognition process by modifying the pathogen identification from an unrecognized immune signature to a recognized immune signature thereby increasing the effectiveness of the immune response to the hidden and/or otherwise unrecognized infection. For example, the immune alerting nanoparticle enters a cells and attach to HIV viruses within an HIV infected cell and displays a set of one or more immune recognizable antigen compound. Said immune recognizable antigen compound incorporates any suitable means necessary to translocate to the surface of the cell to subsequently identify the cell as having a known infection present. Whereby the hidden virus is modified to be recognized as a known virus prior to the macrophage interaction or cell lysis which would release the virus. The translocated recognizable antigen then stimulates immune responses, including, but not limited to, a macrophage interaction. The immune alerting nanoparticle incorporates any suitable means capable of providing the method to increase the effectiveness of a phototherapy, absorbing the energy necessary to create a photodynamic reaction including, but not limited to, the catalytic breakdown of the hidden virus into components that are absorbed by the cell in normal functions, and the absorption of photons capable of causing a carbon nanotube region of the immune alerting nanoparticle to explode in a manner that destroys the HIV virus and/or the cell containing the hidden virus. The preferred embodiments of the present invention provide a suitable means necessary to work with one or more infection types including, but not limited to, hidden infections within a cell that would otherwise be recognized by the immune system outside of a cell, unrecognized infections outside a cell, and unrecognized infections hidden in a cell. Said immune alerting nanoparticle incorporates any suitable means to provide a quorum sensing method of alerting the immune system into an immune response. Wherein said quorum sensing method of alerting provides a threshold condition that would be met before triggering an assisted immune response.

The preferred embodiments of the present invention incorporate any suitable means to provide the method of optionally utilizing cellular elements including, but not limited to, a mitochondrion. The preferred embodiments of the present invention optionally utilizes cellular elements including, but not limited to, glucose, amino acids, adenosine triphosphate (“ATP”), adenosine diphosphate (“ADP”), adenosine monophosphate (“AMP”) nitrogen, or other processes or mechanisms in the body/cell to power the particle or to aid in the particle performing the specific function of the particle.

The preferred embodiments of the present invention optionally incorporate any suitable means to provide the method of sacrificing cells, including, but not limited to, infected or otherwise useless cells in order to harvest components and compounds from the cell to be used in the therapy or the action of the phototherapy functions. Wherein said useless cells include, but are not limited to, fat cells. Wherein said useless cells include, but are not limited to, cell components.

The preferred embodiments of the present invention will optionally be designed so that the inside of the particle contained specific electrical or chemical gradients and a semi or fully permeable membrane to allow the particle to draw in specific chemicals, ions, proteins or any other desired substances to enable the particle perform the function of the particle. The particle incorporates any suitable means capable of providing useful methods, including, but not limited to, sensing chemical gradients to provide alerting methods.

The preferred embodiments of the present invention is comprised of particle types and separate particles being delivered to the body with one or more functions including, but not limited to, an infection locator-type particle. Wherein said infection locator-type particle is comprised of components including, but not limited to, an internal cell specific infection locating means is used to locate a specific pathogen, compound, particle or other desired component within a tagged cell or tissue and said infection locator-type particle incorporate a signal generating means. Said signal generating means has a combination of one or more capabilities including, but not limited to, generate a signal that would either allow the immune system to recognize said tagged cell with the hidden infection, or generate a signal that allows a separate nanoparticle to locate said tagged cell with the hidden infection, tissue or any other suitable target and perform a desired action on said tagged cell, and tissue.

Said manufactured nanoparticle technology would be used alone or in combination with ultraviolet light technology providing the useful method of a photodynamic therapy for patients including, but not limited to, HIV/AIDS patients.

An alternative embodiment of the present invention includes a binding domain including, but not limited to, one domain to bind a specific component, chemical, protein, enzyme or any other desired component and another domain to allow for the excretion and elimination of the bound complex of the nanoparticle and the desired target. The binding for excretion procedure used will optionally be initiated by a suitable method including, but not limited to, injecting a bolus of the particles into the blood, and injecting particles or other fluid areas within the body. The following therapy target compounds and diseases are combinations of one or more material compound targets including, but not limited to, a glucose target, a cholesterol target, a lipids target, a toxin target, an infection target, and a malignancy targets.

Wherein said glucose target including, but not limited to, glucose derivatives and/or glucose metabolites. High glucose levels in diseases including, but not limited to, diabetes is controllably decreased utilizing a particle that bound to glucose for useful methods including, but not limited to, facilitated glucose excretion, elimination of glucose from the body or localized volume, to oxidize glucose, and to provide a suitable entropy change. Excretion means and methods are comprised of combinations of one or more elimination compounds including, but not limited to, bile salts and/or acids, and nitrogen-based compounds to be excreted by action from the kidneys and other excretory organs. Wherein said compounds excreted by renal processes are combinations of one or more compounds including, but not limited to, ammonia, and urea. The preferred embodiments of the present invention optionally incorporate any suitable means capable of proving the method to convert unconverted salts back to precursors for use in body functions including, but not limited to, building proteins.

Wherein said cholesterol targets are combinations of one or more targets including, but not limited to, cholesterol derivatives and/or cholesterol metabolites. The preferred embodiments of the present invention incorporate any suitable means that are capable of providing methods including, but not limited to, the useful method of binding and eliminating cholesterol, whereby the reduction of hypercholesteremia is promoted. An additional useful method of binding and eliminating cholesterol is the reduction of other pathological process involving cholesterol, and the reduction of the elimination process to mimic the elimination process of other eliminated compounds including, but not limited to, utilizing binding to bile salts and acids is one but not the only way in which the therapy operates as that is a natural means for eliminating cholesterol and lipids already.

Wherein said lipids targets are combinations of one or more targets including, but not limited to, lipid derivatives, and/or lipid metabolites functioning by binding one or more nanoparticles to the resulting target, whereby the target-nanoparticle aggregation is more likely to be excreted by normal renal processes.

Wherein said toxin targets are combinations of one or more targets including, but not limited to, a toxin derivative, a toxin metabolite, a poison, a drug in too high a concentration, a drug present accidentally, and an undesired compound. The preferred embodiments of the present invention incorporate any suitable means that are capable of providing methods including, but not limited to, eliminating infection targets by binding one or more nanoparticles to the target resulting in the formation of a target-nanoparticle aggregation more likely to be excreted. Said target particle aggregation is more likely to be excreted by normal renal processes.

Wherein said infection targets are combinations of one or more targets including, but not limited to, parasites, viruses, bacteria and/or fungi. The preferred embodiments of the present invention incorporate any suitable means that are capable of providing methods including, but not limited to, binding one or more nanoparticles to the target, whereby the resulting target-nanoparticle aggregation is more likely to be excreted by normal renal processes.

Wherein said malignancy targets are combinations of one or more targets including, but not limited to, melanomas. The preferred embodiments of the present invention incorporate any suitable means that are capable of providing methods including, but not limited to, said malignancy target elimination, and to provide the useful method for malignant cells to be eliminated from the body by binding one or more nanoparticles to the target, whereby the resulting target-nanoparticle aggregation is more likely to be excreted by normal renal processes.

The preferred embodiments of the present invention incorporate any suitable means that are capable of providing methods including, but not limited to, target disease, and infection-fighting. Wherein said infection-fighting means has the capability of targeting infections including, but not limited to, viral infections, hidden viral infections, and hidden viral HIV infections. The preferred embodiments of the present invention incorporate means that allow for the disruption of the life cycle of the infectious agent and allow for improved healing of the body. Wherein said infectious agent has attributes including, but not limited to, invading viruses, and invading retro-viruses.

Target specific pathogens are combinations of one or more targets including, but not limited to, viruses, bacteria, fungi and/or parasites. Target specific pathogens are more likely to infect a favored tissue and the cells of the favored tissue to infect. Therefore, it is useful to locate said nanoparticles to activate functions of those tissues utilizing binding domains and target only those cells and tissues be infected with a specific pathogen. The particle will optionally then be used to either expose the pathogen to the immune system or perform a specific function to help the body fight the infection using the native and innate immune responses. The preferred particles include, but are not limited to, an antigen presenting tagging particle, an immunofluorescent particle, a reflective particle, a radio-opaque particle, and a selective-cell locating particle. Wherein said preferred particles are compounds capable of locating cells involved with the target disease in order to promote the therapeutic effect.

The preferred embodiments of the present invention makes use of a combination of one or more of the alternative embodiments of the present invention. The preferred embodiment of the present invention typically includes phototherapy embodiments, and photodynamic therapy means. Wherein said photodynamic therapy means is comprised of components including, but not limited to, UVA-LED, UVA1-LED, UVA1C-LED, and psoralen compound. Said photodynamic therapy means has capabilities including, but not limited to, photo-activation of chemical compounds, photo-deactivation of a chemical compound. The preferred embodiments of the present invention make use of the photodynamic therapy capabilities to increase the probability of chemical reactions leading to a therapy prescription delivery. One or more LED types will be provided to provision the photodynamic therapy as required by the photodynamic therapy prescription.

The tenth alternative embodiment of the present invention has a nanoscale form factor of the preferred embodiments of the present inventions will optionally also be manufactured partially complete and injected only to be completed after placement within the body once the particle gathers specific cellular, sub-cellular or body components to make the particle functional. Wherein said sub-cellular components including, but not limited to, utilizing a mitochondrion for energy, harnessing the enzymes and killing power present within a lysosome or macrophage or drawing in of other compounds including, but not limited to, molecules, amino acids, proteins, and DNA.

The nanoparticle form factor of the preferred embodiments of the present invention will optionally be used in combination with a phototherapeutic source to trigger an immune response of the target cells. The nanoparticle form factor of the preferred embodiments of the present invention will incorporate combinations of one or more cell test actions and measurement sensors. Wherein said test actions include, but is not limited to, a temperature tests, a pressure tests, a mass tests, a chemical tests, a spectral tests, an acoustic tests, a pressure tests, a flow rate test, a pH tests, an ion concentration test, an electrical resistance over a range of frequencies test, a cell size tests, a surface properties test, a chemical concentration test, a rate of reaction test, a rate of absorption of thiamine test, a colorimetry test, an independent movement test, and a magnetic resonance test. Wherein said tests are combinations of one or more tests, including, but not limited to, internal, and external tests. Wherein said independent movement test compares motion of self cells to non-self cells for the purpose of identifying non-self pathogens. Independent movement is an indication of the presence of a parasite. Uncharacteristic independent movement is a discriminating test useful for parasite location and identification.

The preferred embodiments of the present invention incorporate any suitable means capable of providing methods to use components of the infected cell to build the immune system component known as “complement”; whereby said complement will subsequently translocate to target other cells, and if additional infections are found to continue the process of using the infected cell to build said complement repeating the translocation process and complement generation until the infection is removed, or until the external signal to stop complement production is received. The preferred embodiments of the present invention incorporates any suitable means to provide the methods capable of generating and responding to phototherapy control signals including, but not limited to, a signal to start, a signal to stop, a signal to change mode, and a signal to halt until additional signals are received, a signal to respond to local control signals, a signal to respond to external controls. An example of said signal to stop is the use of a sequence of wavelengths determined a phototherapy prescription to active the start generating complement method of the preferred embodiment. The use of the start function limits the local generation of complement to the specific locations targeted by the phototherapy prescription. The sequence of light is modulated in temporal and spectral domains.

The preferred embodiments of the present invention incorporate any suitable means capable of providing methods to make use of light internally to set up communication channels within the body that provide methods including, but not limited to, logical nerve networks that effectively enhance nerve components, and link broken or dysfunctional nervous system component connections. The method of using the phototherapy to create a communications network within the body or within a body component, or a cell will provide the useful function of repairing nerve damage, and the useful function of creating additional connections that would otherwise not have existed previously. The communication phototherapy includes any suitable means to create input ports and outputs ports that are compatible with the interface available in the biological target environment. Said alternative nerve networks are capable of receiving signal from external sources. For example an external control system has capabilities to determine control sequences that effect an action in a patient that was useful and compatible with environmental conditions including, but not limited to, assisted sight, assisted walking, and assisted breathing. Said dysfunctional nervous system components are combinations of one or more nervous system components including, but not limited to, neurons, axons, dendrites, synapses, synaptic cleft, synaptic vesicles, neurofibrils, neurotransmitters, Golgi apparatus, ribosomes, mitochondrion, smooth ER, chemical synapses, electrical synapses, myelin sheath, Schwann cells node of Ranvier, and immunological synapse.

### Alternative Embodiment

Photophoresis

Photophoresis incorporating UVA1-LEDs is a novel configuration having advantages over the prior art methods for the reasons enumerated in the preferred embodiments of the present invention. The improvements of the preferred embodiments of the present invention include, but are not limited to, the reduction of the risk to exposure to UVB and UVC wavelengths, and the method of sequencing the spectral irradiance to increase the probability of a chemical reaction.

Said photophoresis system optionally is small enough to fit internal to the body to create an internal separation means for treatments, incorporating any suitable means capable of providing methods of internal photophoresis. Said photophoresis system incorporates any suitable means including, but not limited to a stent, a partially opaque stent, an opaque stent, and a translucent stent.

### Alternative Embodiment

### Micro Channel Cell Specific Phototherapy

A seventeenth preferred embodiments of the present invention provides an improvement to photophoresis methods whereby the cells travel through an array of micro-channels with detectors and emitters on either side that diagnose conditions and take action depending on the diagnosis results, including, but not limited to, cell by cell photodynamic therapy, cell by cell phototherapy, cell by cell chemotherapy, or cell be cell separation. An extension of said photophoresis improvement is to react with specific compounds within a given cell.

### Alternative Embodiment

### External Hand Held Wand Phototherapy

External Hand Held Wand Phototherapy incorporating phototherapeutic LEDs is a novel configuration having advantages over the prior art methods for the reasons enumerated in the preferred embodiments of the present invention. The improvements of the preferred embodiments of the present invention include but are not limited to, the reduction of the risk to exposure to UVB and UVC wavelengths, and the method of sequencing the spectral irradiance to increase the probability of a chemical reaction. Wherein phototherapeutic LEDs are combinations of one or more LEDs having peak wavelengths within ranges including, but not limited to, UVA, UVA1, and UVA1C.

### Alternative Embodiment

### External Partial Body Pad Phototherapy

External Partial Body Pad Phototherapy incorporating phototherapeutic LEDs is a novel configuration having advantages over the prior art methods for the reasons enumerated in the preferred embodiments of the present invention. The improvements of the preferred embodiments of the present invention include, but are not limited to, the reduction of the risk to exposure to UVB and UVC wavelengths, and the method of sequencing the spectral irradiance to increase the probability of a chemical reaction. Wherein phototherapeutic LEDs are combinations of one or more LEDs having peak wavelengths within ranges including, but not limited to, UVA, UVA1, and UVA1C.

### Alternative Embodiment

### Cell Analysis by Refraction Phototherapy

A fifteenth embodiment of the present invention uses of at least two light emitting devices that are on either sides of a cell wherein the position of the devices changes based on natural Brownian motion or active positioning to include a diagnostic method to emit photons of a diagnostic method, emit electromagnetic frequencies of a communication method, and emit a phototherapeutic flux. The useful method of using a light refraction material or a composite emitter and detector pair device is to reduce collateral cell component damage and optimize the energy efficiency for the primary method of targeting pathogens and diseased tissues, while avoiding healthy tissue as much as possible. The preferred embodiments of the present invention incorporate any suitable means capable of providing a method to first emit diagnostic light with a substantially non-hazardous wavelength to detect photosensitive components of target cells along a diagnostic axis, and a second wavelength of substantially phototherapeutic wavelength for action along said diagnostic axis.

### Alternative Embodiment

### Cell Analysis by Reflection Phototherapy

A sixteenth preferred embodiments of the present invention incorporates any suitable means capable of providing combinations of one or more methods including, but not limited to, creating a light chamber around a cell, creating a reflective surface on a portion of the cell, creating a reflective surface adjacent to the cell wall. Wherein said reflective surface adjacent to the cell wall reflects light passing through the cell back through the cell. The fifteenth embodiment of the present invention incorporates any suitable means to provide the method of diagnosing a cell condition. Wherein said method of diagnosing a cell condition is a combination of one or more suitable methods including, but not limited to, emitting a first non-harmful diagnostic photon toward a cell, whereby said reflective surface reflects the coded photon stream back to the detector for analysis, whereby spectral signatures are detected thereby initiating a trigger condition, whereby said trigger condition emits a subsequent phototherapeutic photon flux is emitted toward the targets that triggered the effective use of the phototherapeutic emissions. The useful method of the cell-by-cell diagnostics is the reduction in the application of phototherapeutic photons on cells that do not require a phototherapeutic effect. Said reflective diagnostic means may also be used in combination with fluorescent nanoparticles to provide a phototherapy for cell-by-cell basis. The diagnostic methods are optimized to minimize energy use and generally use the least powerful combination of wavelengths to make a suitable determination of the predicted usefulness of a triggered phototherapeutic flux emission, incorporating combinations of one or more parameters of phototherapeutic effect including, but not limited to, spatial orientation parameter, spectral irradiance parameter, flux power density parameter, and optical parameters. Additional suitable means are incorporated capable of providing methods to provide data collection on diagnostic findings, phototherapy actions, and diagnostic results. Said diagnostic capabilities are used before, during and after said phototherapeutic action is performed. Data is collected at all stages of the phototherapeutic procedure and communicated to an external control computer incorporating a patient records database. Said data is subsequently used to improve the phototherapy for subsequent phototherapeutic actions in the current phototherapy session and in subsequent phototherapy sessions on the same patient, and for other patients that display at least one or more similarities to the current patient. The diagnostic wavelengths are generally longer than the phototherapeutic wavelengths. A preferred phototherapy wavelength is generally less than or equal to half of the absorbed diagnostic wavelength. A spectrometer is used to measure the wavelength of re-emitted light to determine the fundamental frequency of the absorbing electron to potentially identify the absorbing compound in a manner similar to spectroscopy. A set of re-emission data for a given compound can increase the accuracy of a nano-scale spectroscopy classification test. Wide spectrum diagnostic wavelengths can be used to determine which wavelengths are absorbed and which wavelengths are reflected, and which wavelengths do not interact. The diagnostic spectral irradiance includes any suitable means to provide a combination of one or more diagnostic methods including, but not limited to, a wide range dynamic peak, a narrow dynamic peak, and a dynamic full width half maximum, a dynamic power flux density, dynamic target spatial tracking, and dynamic optical parameters. Likewise, the phototherapeutic spectral irradiance includes any suitable means to provide a combination of one or more diagnostic methods including, but not limited to, a wide range dynamic peak, a narrow dynamic peak, and a dynamic full width half maximum, a dynamic power flux density, a dynamic target spatial prediction, and a dynamic optical parameters.

### Alternative Embodiment

### Cell Isolating Phototherapy

A seventeenth preferred embodiments of the present invention incorporates any suitable means capable of providing methods of cell interaction including, but not limited to, isolating at least one cell, linking multiple cells into an aggregate shapes, de-isolating at least one cell, and de-aggregating multiple cells. Wherein said aggregate shape is a shape that provides a combination of one or more useful methods, including, but not limited to, isolating at least one cell, isolating at least one cell component, isolating an antigen displaying pathogen, isolating a virus, isolation a hidden virus, isolating HIV, isolating variants of HIV. The useful purpose of the isolation is to prepare a cell and/or aggregation of cells for a phototherapeutic action.

### Alternative Embodiment

### Electroluminescent Nanoparticle Low-pressure Electric Discharge Lamp

A eighteenth embodiment of the present invention is comprised of an electrical discharge lamp with a diffused nanocrystal dust suspended internal to the lamp envelope in which the electrons emitted from the anode accelerate across the electric potential and prior to reaching the cathode has a high probability of impinging on said diffused nanocrystal dust elevating the energy of orbital electrons in said nanocrystal and precipitating an photonic emission from the elevated energy nanocrystal. Said eighteenth embodiment is a combination of one or more suitable means including, but not limited to, a modified low-pressure fluorescent bulb, and a modified high-pressure vapor bulb. Wherein said modified low-pressure fluorescent bulb incorporates combinations of one or more vapor compounds including, but not limited to, mercury, a nanocrystal, a zinc oxide based nanocrystal, and a titanium oxide based nanocrystal. The nanocrystal dust provides the useful method of converting electric potential into a phototherapy ready photonic emission that is more useful than mercury vapor fixed elemental discrete spectral lines. Said phototherapy ready photonic emission is suitable for phototherapeutic uses, and may also be partially converted to additional wavelengths by phosphor converting compounds prior to reaching the phototherapy patient. Said diffused nanocrystal is suspended by combinations of one or more methods including, but not limited to, active nanocrystal suspension methods, and passive nanocrystal suspension methods. Said active nanocrystal suspension methods include combinations of one or more suspension methods, including but not limited to, mechanical vibration, liquid vibration, gaseous vibration, pumping, spraying, and dynamic magnetic flux coupling. Said passive nanocrystal suspension methods include combinations of one or more suspension methods, including but not limited to, non-stick internal wall material, Brownian motion enhancements, and temperature gradients.

### Alternative Embodiment

### Combination Phototherapy

A nineteenth embodiment of said present invention is comprised of combinations of one or more preferred embodiments of the present invention including, but not limited to, a phototherapy effectiveness measurement means. Wherein said phototherapy effectiveness measurement means incorporates one or more suitable diagnostic means including, but not limited to, a reflectometer means, a spectroscopic means, an imaging means, a remote control means, and a colorimeter means. Said diagnostic means provides the useful method of medical personnel providing a determination for a phototherapy prescription based on the presented results of the analysis and findings of said diagnostic means. For example, the preferred embodiments of the present invention may include any suitable phototherapy effectiveness measurement means to optimize the phototherapy. The authorized medical personnel can review the results and suggest or approve system suggested modifications to the phototherapy prescription including, but not limited to, modifying the location of the phototherapeutic effect. As the preferred embodiments of the present invention move relative to the phototherapy treatment site, or the phototherapy site move dynamically relative to the preferred embodiments of the present invention, or combinations of one or more relative movement including, but not limited to, the phototherapy targets change dynamically to meet a phototherapy prescription, and the phototherapy changes to meet a modified phototherapy prescription. Phototherapy control decisions are made using inputs from phototherapy components including, but not limited to, said phototherapy effectiveness measurement means, control phototherapy, control drug delivery, control effectiveness means, control data input, communications means, data output, database, memory management, logical groups, accuracy of target phototherapy, and optimized energy efficiencies.

### Alternative Embodiment

Capture Capsule Therapy with Phototherapeutic Capabilities

A twentieth embodiment of the present invention incorporates any suitable means capable of providing the methods to allow a capsule to absorb other material including, but not limited to, another capsule, pill, chemical, and compound for elimination or for redeployment further down the gastrointestinal tract.

### Alternative Embodiment

### LED Cancer Therapy Device

A twenty-first embodiment of the present invention incorporates any suitable means to provide a method of accessing an intra-body site to provide methods including, but not limited to, phototherapy methods, and photodynamic therapy methods. The preferred embodiments of the present invention incorporate any suitable means with the capabilities to provide an intra-body light source within an intra-body site. Wherein said intra-body site is a combination of one or more site including, but not limited to, the intrathecal site, a blood vessel site, an abdominal site, and a bone marrow site. Wherein said intrathecal site is also known as the intrathecal space. Wherein said intrathecal space is the sub-dural space of the spinal cord. Said intra-body light source is comprised of combinations of one or more light source types including, but not limited to, an LED, a UV-LED, a UVA1-LED, and a UVA1C-LED. Said twenty-first embodiment of the present invention incorporates several useful phototherapeutic methods, including, but not limited to, a light-activated cancer drug activation, and a location specific light-activated drug activation.

Said light-activated cancer drug activation methods are comprised of methods including, but not limited to, first injecting said light-activated drug into said body site, and subsequently activating said light-activated drug within the vicinity of a cancer cell at a prescribed rate to provide the useful purpose of modifying that cell from said intra-body site. Wherein said cancer cell is of cancer cell types including, but not limited to, a leukemia cell, a B-cell type lymphoma cell, and a T-cell type lymphoma cell. Said light-activated cancer drug activation methods includes multiple sequencing of light-activated drug of the same or varying drug types. Wherein said varying drug types includes, but is not limited to, light-activating drug neutralizing compounds. Said light-activated cancer drug activation methods includes the method of removing excess blood, removing excess material and/or removing processed drug materials as needed to optimize the approximation of said phototherapy prescription. Said phototherapy prescription parameters are combinations of one or more phototherapy parameters including but not limited to, pressure gradients in said body-site.

The preferred embodiment of the present invention incorporates any suitable target cell locating means capable of providing the useful method of injecting a bolus of one or more combinations of a target cell binding drugs into said body-site. Said target cell binding drug provides the useful method of locating one or more target cells by binding to a specific target cell type. Said target cell binding drug is comprised of any suitable means capable of providing target cell locating methods including, but not limited to a monoclonal antibody for a specific type of target cell, a nanoparticle, and a carbon nanotube. Wherein said target cell locating means that have target specific binding domains for combinations of one or more phototherapy targets including, but not limited to, a photodynamic drug, and a target cell of interest. Wherein said phototherapy targets are combinations of one or more targets including, but not limited to, said target cell type, a component of the surface of said target cell, a component within said body-site, and fluid within said body-site. Wherein said fluid within said body-site include combinations of one or more compounds including, but not limited to, a protein, a sugar, a fat, a by-product of said target cell, and a target cell component. The preferred embodiment of the present invention incorporates any suitable means capable of providing the useful method of injecting said target cell binding drug as a sequence of one or more separate compositions. The sequence of separate compositions subsequently reacts within said body-site. Said light-activated drug reacts with said target cell binding drug to bind both said light-activated drug and said target cell creating a composition of matter referred to herein as a complete binding complex. Said complete binding complex enables un-activated molecules of said light-activated drug to indirectly attach to a target cell, prior to activation of said light-activated drug. Wherein said desired target cell includes, but is not limited, to a leukemia cell, a B-cell type lymphoma cell, a T-cell type lymphoma cell, said target cells, and cell components. The present invention incorporates any suitable means capable of providing the useful method of activating said complete binding complex by operational LED device said light-activated drug is activated within a close enough proximity to the target to act substantially only upon the target cell.

The locating component of said complete binding complex incorporates any suitable elimination means capable of providing a method to degrade or otherwise become non-antigenic disposable elements following activation and delivery effect of said light-activated drug. Wherein said suitable elimination means is comprised of combinations of one or more means, including, but not limited to, an elimination domain. Wherein said elimination domain becomes active by internal or external stimulus preferably after said light-activated drug delivered the intended photodynamic therapeutic effect. Said elimination domain is previously described above in this document.

The methods of using said complete binding complex allow for targeted cell killing in target diseases including, but not limited to, leukemia and/or lymphoma. In said target diseases, therapeutic agents currently used to treat the diseases do not preferentially kill cancerous cells but instead preferentially target the faster dividing cells. While said faster dividing cells are more likely to be a cancerous cell than normal dividing cells, some non-cancerous cells are also known to be of said faster dividing cell type including, but not limited to, hair follicles, cells lining the gastrointestinal tract, and rapidly dividing cell populations within the human body. The preferred embodiments of the present invention incorporate any suitable means capable of providing methods to attach said un-activated light-activated drug only to said target cells, and to activate the un-activated light-activated drug using a controlled activation system such as a LED which allows selective action upon only said target cells. Said controlled activation system provides the useful method of activation while reducing the collateral damage of non-target disease cells death, and reducing ancillary tissue death that typically occurs in current therapies including, but not limited to, chemotherapy and radiation.

### Alternative Embodiment

### Specific Cancer Therapy for Leukemia/Lymphoma

A twenty-second embodiment of the present invention incorporates any suitable means to provide a method to address direct problems in the treatment of said target disorders and all of the target disorders sub-types, specific therapies would be most beneficial when delivered into the Leukemia and Lymphoma phototherapy sites which is comprised of combinations of one or more sites including, but not limited to blood sites, bone marrow sites, cerebral spinal fluid within the spinal column sites, ventricles sites, and the lymphatic system sites. Wherein said lymphatic system sites is comprised of combinations of one or more sub-sites including, but not limited to, lymph nodes sites, thymus sites, spleen sites, lymphatic drainage sites, and lymphatic communication systems sites.

The preferred embodiment of the present invention incorporates any suitable means providing the method of implanting an LED within said Leukemia and Lymphoma phototherapy sites, such as intrathecally. The preferred embodiment of the present invention incorporates any suitable means capable of providing the method of injecting combinations of one or more Leukemia and Lymphoma phototherapy components into said Leukemia and Lymphoma phototherapy sites to provide the useful therapeutic effects including, but not limited to, diffusion of said Leukemia and Lymphoma phototherapy components to the target disease tissue, an adequate volume for distribution of said Leukemia and Lymphoma phototherapy components to the target disease tissue, and proximal location of said Leukemia and Lymphoma phototherapy components to the target disease tissue. Wherein said Leukemia and Lymphoma phototherapy sites are comprised of sites including, but not limited to the cerebral spinal fluid space. Wherein said Leukemia and Lymphoma phototherapy components is comprised of components including, but not limited to, a bolus of drug component, diagnostic enhancement component, and locating component. Subsequent to the method of injecting combinations of one or more Leukemia and Lymphoma phototherapy components into said Leukemia and Lymphoma phototherapy sites, the natural flow dynamics of these fluids, activation of the LED with the wavelength(s) necessary to activate said Leukemia and Lymphoma phototherapy components to act on target disease tissue within the volume of said Leukemia and Lymphoma phototherapy sites within suitable intrathecal Leukemia and Lymphoma phototherapy prescription parameters. Wherein said intrathecal Leukemia and Lymphoma phototherapy prescription parameters are combinations of one or more phototherapy parameters including but not limited to, a suitable period of time dependent on the flow dynamics of said Leukemia and Lymphoma phototherapy sites. The intrathecal Leukemia and Lymphoma phototherapy prescription parameters are determined using known scientific data on the flow dynamics of said Leukemia and Lymphoma phototherapy sites, and on a titrated volume of said Leukemia and Lymphoma phototherapy components required to treat substantially all of said Leukemia and Lymphoma phototherapy site adequately resulting in improved target cell kill in diseases such as leukemia and lymphoma while delivering a therapy with a reduced side-effect profile and a reduced additional healthy tissue damage.

The term intrathecally is an adjective describing a site known to contain the cerebral spinal fluid.

### Alternative Embodiment

### Drug Delivery Phototherapy Capsule

A twenty-third embodiment of the present invention incorporates any suitable means to provide a method to deliver drugs with a prescribed phototherapy, wherein said drugs are dynamically delivered at a suitable location and for a suitable duration. A first set of drugs are delivered prior to the phototherapy, a second set of drugs is delivered during the phototherapy, and a third set of drugs are delivered after the phototherapy.

### Alternative Embodiment

### Phototherapy Control System

The preferred embodiments of the present invention have a temporal response time that is limited by the LED turn on and LED turn off times. The LED turn on and LED turn off times vary from LED type to LED type, and vary to a lesser degree between LEDs of the same type. The control system will be selected to be able to control the LEDs timing to within fraction of the LED response times. The control system will store the LED characteristic response times in any suitable means that allows for the control system to optionally track the phototherapy prescription with the least amount of error.

### Alternative Embodiment

### Phototherapy Incorporating Optical Spectral Filter Management System

A twenty-fifth embodiment of the present invention incorporates any suitable means to provide a method to provide optical spectral filter management system, is comprised of combinations of one or more components including, but not limited to, long wavelength pass spectral filter, band pass spectral filter and low pass filter. Said optical spectral filter means incorporates dynamic functions including, but not limited to, dynamic spectral tracking system, mechanical rotation, and mechanical translation.

### Description—Alternative Embodiment

### Phototherapy Chamber

A twenty-sixth embodiment of the present invention incorporates any suitable means capable of providing a method to a phototherapy chamber incorporating phototherapeutic LEDs. Wherein said phototherapeutic LEDs are comprised of combinations of one or more LED types including, UV-LED, UVA-LED, UVA1-LED, and UVA1C-LED. Wherein said phototherapy chamber is capable of providing useful methods including, but not limited to, optimizing a phototherapy prescription for said target diseases, and optimizing a photodynamic therapy for said target diseases.

### Alternative Embodiment

Phototherapy with Dual Purpose Light Source

A twenty-seventh embodiment of the present invention incorporates any suitable means capable of providing a method to use the LEDs for the dual purpose of photonic communications and of phototherapy. The communications networks use the flux modulation capabilities of the phototherapeutic LEDs to communicate information to a phototherapy flux demodulation means. Wherein said phototherapy flux demodulation means is comprised of any suitable means capable of providing the function of demodulating said modulated phototherapy flux including, but not limited to, a optical spectral filter, an optical polarizing spectral filter, a photodetector, a photodiode, phototransistor, and an optocoupler.

### Alternative Embodiment

### Optoelectronics Enhanced Nervous System Phototherapy

A twenty-eighth embodiment of the present invention incorporates any suitable means capable of providing the method to enhance the nervous system of the patient. The enhancement to nervous system includes the creation of optoelectronic links from a first nerve component to a second nerve component. Said first nerve component and said second nerve component have a normal nerve component pair relationship. Said normal nerve component pair relationship is subject to patient aging and failure over time. The useful method of the present invention allows the enhancement of nerve pairs including, but not limited to, restore dysfunctional nerve pair relationships, and enhance non-functional nerve pair relationships. Said nerve component is comprised of combinations of one or more cell components including, but not limited to, dendrites, and axons. Said optoelectronic nerve interface is comprised of combinations of one or more interface components including, but not limited to, sodium gradient simulator, and sodium gradient detector. Said sodium gradient also uses a electronic capacitance means to simulate a natural sodium gradient stimulation relaxation cycles.

A set of nanoscale optoelectronic nerve interface particles (“NONIP”) is released into the general region of the nerve dysfunction. The set of NONIPs then aggregate on nerve components and create a distributed peer to peer communications network. A suitable distributed mapping algorithm determines the best routes to create a link between a first nerve component and a second nerve component in the useful process of enhancing nervous system function. The NONIP incorporates combinations of one or more NONIP components including, but not limited to, photonic emitter, and photonic detectors.

The optoelectronic enhanced nervous system phototherapy has capabilities to operate with the presence of high flux of photons such as that of a phototherapy of other ambient lighting conditions in which the patient resides. The phototherapies that are compatible with the optoelectronic enhanced nervous system phototherapy do not modulate the phototherapy emitters in the frequency range of the optoelectronic enhanced nervous system phototherapy in order to avoid interference. Device registration procedures are incorporated to announce the presence of an optoelectronic communication method so that compliant devices can establish a common protocol to communicate, and a protocol to avoid disruption of service. Multiple carrier wavelengths are used to establish multiple communication channels, and multiple modulation frequencies of the carrier wavelengths are used to further establish multiple communication channels. UV is not normally present indoors, and patient UV protective clothing also reduces the UV stray incidence creating the conditions that would allow the spurious and stray UV to be treated as background noise in said optoelectronic enhanced nervous system phototherapy methods.

The preferred wavelengths of light used to communicate are dependent on multiple conditions including, but not limited to, distance, composition of medium, bandwidth, and negotiated protocol compatibility.

The phototherapy described herein provides the useful benefit of a therapeutic enhancement of a nervous system.

The preferred embodiments of the present invention incorporate any suitable means capable of providing the method of creating regions of activities within the patient. Said regions of activities vary dynamically to provide a therapeutic prescription. The preferred embodiments of the present invention incorporate any suitable means capable of providing useful methods including, communications to control independent regions of activity, a mesh network with spatial resolution. The enhanced nervous system is controlled to provide enhanced health improvements including, but not limited to, immune system stimulation, hematopoiesis stimulation, renal function control, and target disease control.

### Alternative Embodiment

### Phototherapy Incorporating Magnetic Field

The preferred embodiments of the present invention incorporate any suitable magnetic field means capable of providing the methods including, but not limited to, promoting desirable chemical reactions, and inhibiting undesirable chemical reactions. Wherein said magnetic field means incorporates combinations of one or more components including, but not limited to, alternating magnetic field generator, magnetic resonance imaging system, static magnetic field, polarized phototherapy spectral irradiance synchronized with magnetic field means. Wherein said undesirable chemical reactions include, but is not limited to, the generation of hydrogen peroxide, and oxidative chemical species. Wherein said desirable chemical reactions include, but is not limited to, the elimination of hydrogen peroxide, and oxidative chemical species.

### CONCLUSION

While the above description contains many specificities, these should not be construed as limitations on the scope of the preferred embodiments of the present invention, but as exemplifications of the presently preferred embodiments thereof. Many other ramifications and variations are possible within the teaching of the preferred embodiments of the present invention. All patents, applications and articles mentioned herein, including U.S. patent applications Ser. No. 10/591,960 filed on Mar. 9, 2005, and U.S. Ser. No. 10/558,092 filed on May 24, 2004, and U.S. Ser. No. 10/714,824 filed on Nov. 17, 2003, are incorporated herein by reference in their entirety.

The definitions of the terms UV, UVA, UVA1, UVA1C, UVA2, UVA3, UVB, and UVC vary in the literature to suit each authors needs. Therefore, the terms UV, UVA, UVA1, UVA1C, UVA2, UVA3, UVB, and UVC are understood to be variables defined in the context of each reference separately. The terms UV, UVA, UVA1, UVA1C, UVA2, UVA3, UVB, and UVC and are not always suitable for direct comparisons between references. To harmonize references, the terms UV, UVA, UVA1, UVA1C, UVA2, UVA3, UVB, and UVC are to be translated into wavelength in nanometers of a photon in a vacuum.

The following references are incorporated herein by reference in their entirety with the exclusion of the definitions of the terms UV, UVA, UVA1, UVA1C, UVA2, UVA3, UVB, and UVC:

1. McGrath H Jr, Bak E, Michalski J P. Ultraviolet-A light prolongs survival and improves immune function in (New Zealand black New Zealand white) F1 hybrid mice. Arthritis Rheum 1987; 30:557-61.

2. McGrath H Jr. Ultraviolet-A1 irradiation decreases clinical disease activity and autoantibodies in patients with systemic lupus erythema-tosus. Clin Exp Rheumatol 1994; 12:129-35.

3. McGrath H Jr, Martinez-Osuna P, Lee F A. Ultraviolet-A1

(340-400 nm) irradiation therapy in systemic lupus erythematosus. Lupus 1996; 5:269-74.

4. Molina J F, McGrath H Jr. Longterm ultraviolet-A1 irradiation therapy in systemic lupus erythematosus. J Rheumatol 1997; 24:1072-4.

5. Polderman M C A, Le Cessie S, Huizinga T W J, Pavel S. Efficacy of UVA1 cold light as an adjuvant therapy for systemic lupus erythematosus. Rheumatology 2004; 43:1402-4.

6. Vila L M, Mayor A M, Valentin A H et al. Association of sunlight exposure and photoprotection measures with clinical outcome in systemic lupus erythematosus. P R Health Sci J 1999; 18:89-94.

7. Kumar, V, Abbas, A K, Fausto, N. Pathologic Basis of Disease, 7th ed. Philadelphia, Pa.: Elsevier Saunders; 2005: 441-442.

8. Andrade F, Casciola-Rosen A, Rosen A. Apoptosis in systemic lupus erythematosus: clinical implications. Rheum Dis Clin North Am 2000; 26:215-7.

9. Andrade F, Casciola-Rosen L A, Rosen A. Generation of novel covalent RNA-protein complexes in cells by ultraviolet B irradiation. Arthritis Rheum 2005; 52:1160-70.

10. Hasan T, Nyberg F, Stephansson E et al. Photosensitivity in lupus erythematosus, UV provocation results compared with history of photosensitivity and clinical findings. Br J Dermatol 1997; 136:699-705.

11. Polderman M C A, Huizinga T W J, Le Cessie S, Pavel S. UVA1 cold light treatment of SLE: a double blind, placebo controlled crossover trial Ann Rheum 2001; 60:112-5.

12. Szegedi A, Simicx, A M, Horkay I et al. Ultraviolet-A1 phototherapy modulates Th1/Th2 and Tc1/Tc2 balance in patients with systemic lupus erythematosus. Rheumatology 2005; 44:925-31.

13. Zane C, Leali C, Airo P, Panfilis G, Pinton P C. High-dose UVA1 therapy of widespread plaque type, nodular, and erythrodermic mycosis fungoides. J Am Acad Dermatol 2001; 44:629-33.

14. Polderman M C A, Van Kooten C, Smit N P M, Kamerling S W A, Pavel S. UVA1 radiation suppresses immunoglobulin production of activated B-lymphocytes in vitro. Clin Exp Immunol 2006 (in press).

15. Jacobi A M, Odendahl M, Reiter K et al. Correlation between circulating CD27high plasma cells and disease activity in patients with systemic lupus erythematosus. Arthritis Rheum 2003; 48:1332-42.

16. McGrath H Jr. Elimination of anticardiolipin antibodies and cessation of cognitive decline in a UV-A-1-irradiated systemic lupus erythematosus patient. Lupus 2005; 14:859-61.

17. Burren R, Scaletta C, Frenk E, Panizzon R G, Applegate L A. Sunlight and carcinogenesis: expression of p53 and pyrimidine dimers in human skin following UVA I, UVA I [symbol] II and solar simulating radiations. Int J Cancer 1998; 76:201-6.

18. Menon Y, McCarthy K, McGrath H Jr. Reversal of brain dysfunction with UV-A1 irradiation in a patient with systemic lupus. Lupus 2003; 12:479-82.

19. Kindt, T J, Goldsby, R A, Osborne, B A. Kuby Immunology, 6th ed, New York: W. H. Freeman and Company; 2007.

### Examples

Effects of LED on Hippocampal Neuron Survival and Morphogenesis In Vitro

Injury or degenerative conditions affecting the brain, spinal cord or peripheral nerves have a devastating impact on the quality of life for affected individuals. The experiments are focused on testing the safety and efficacy of LED phototherapy that interfaces with neurons and stimulate de novo or regenerative neuronal development. The data obtained contributes to the optimization of phototherapy devices designed for this purpose, as an example of device designs that enhance basic science investigation of the mechanism of LED effects on neural cell functions, such as dendritic remodeling, synaptic plasticity, and resistance to injury, ischemia, toxic environmental agents or metabolic conditions that are otherwise damaging to neurons. The LEDs used in this experiment included UV-LEDs.

New technologies to modulate morphogenesis, such as to promote morphogenesis, including aiding nerve regeneration or de novo development of neural cell transplants require that phototherapy devices be designed both to support neuronal growth at various stages of neural cell maturation and to elicit specific responses from particular classes of neurons. Specificity is gained by tailoring the parameters of LED exposure to complement the inherent properties of cellular responses. For example, pyramidal neurons undergoing axonogenesis during early stages of development are optimally stimulated to grow when LED exposure adheres to one set of parameters.

These experiments utilize low-density primary cultures of fetal rat hippocampal neurons (Kaech and Banker, 2006) as a model system for investigating the effects of LED exposure on the development and function of neurons from the central nervous system (CNS). These cultures are the most extensively characterized primary culture of mammalian CNS neurons and are unparalleled in the number of complimentary studies in situ. Neurons in these cultures (i) are nearly homogeneous-typically 94% are readily distinguished as pyramidal and the rest are interneurons, (ii) survive for up to 4 weeks, undergoing a stereotypical, nearly synchronous development of a single axon and several dendrites of well-defined shape and characteristic molecular constituents, and (iii) form synaptic relationships that are representative of those normally present in the hippocampus. This parallels hippocampal development in situ, which is largely complete by the end of the third postnatal week.

### Overview of Experimental Design and Analysis:

The experiments are designed to test two variables of exposure and interactions between them; wavelength, and intensity relative on neuronal survival and development. For example, some cultures received LED exposure continuously beginning shortly after plating for 24 hours. The time points for morphometric analyses after LED exposures have been selected based on the known pattern of neuromorphogenesis in hippocampal cultures (Goslin and Banker, 1991; Fletcher et al., 1994).

In the experiments, the neurons in hippocampal cultures were exposed to light from a single LED in modular chambers (“pods”) designed and constructed and placed in a HEPA-filtered CO2 incubator to maintain cultures in a humidified environment at 36° C. and 5% CO2 without exposure to room or outdoor light. The pods permitted testing of up to 10 different exposure paradigms in duplicate per experiment. Table 1 illustrates the range for the irradiance power signal in accordance with an aspect of this invention. The wavelength and estimated, power intensity and total energy density for each condition tested was blinded from the experimenter to prevent bias. In each experiment control cultures were maintained in the same incubator and culture media but were not exposed to any LED. At the end of the exposure period, the cultures were fixed and stained for assessment of toxicity and effects on early stages of neuronal development. Specific end points measured to date include neuronal survival and apoptosis, rate of initial polarization of the cell into axonal and somatodendritic domains, and axon length at 1 day in-vitro.

TABLE 1
Pod Specs

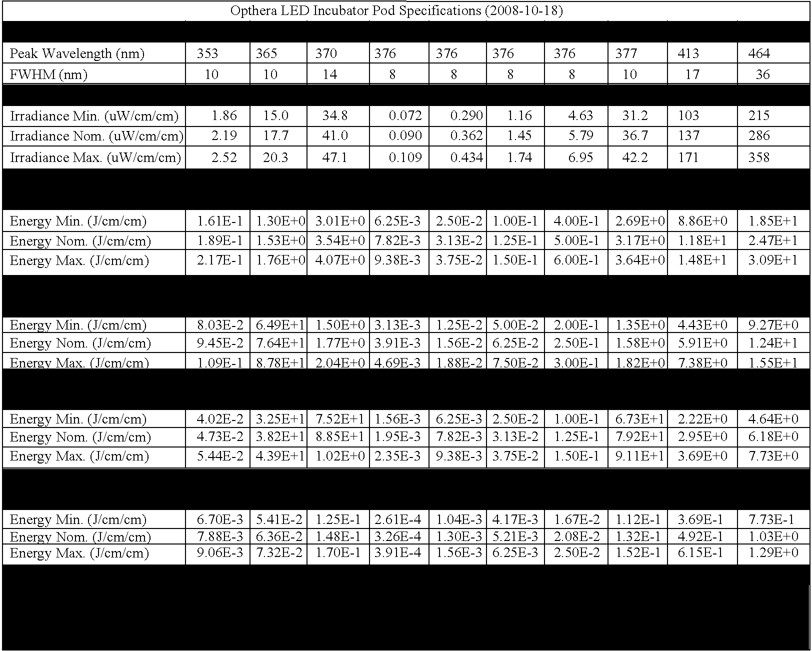

### Detailed Experimental Methods:

### Neuronal Cell Culture Methods:

The methods for preparing hippocampal cell cultures, and for distinguishing pyramidal neurons, which make up about 94% of the cells in these cultures, from GABAergic interneurons and non-neuronal cells, are described in [Clamp and Lindsley, 1998; Yanni and Lindsley, 2000]. Briefly, neuron cultures were prepared from hippocampi of fetal Sprague-Dawley rats (Taconic Farms) at gestational day 19, as described by [Kaech and Banker, 2006]. Briefly, hippocampi were dissected from the cerebral hemispheres of 6-9 fetuses, the meninges removed, collected them in HEPES-buffered salt solution, dissociated the cells with trypsin (0.25% for 15 min at 37° C.) and triturated with a fire-polished Pasteur pipette. The cells were plated in Minimal Essential Medium (MEM) with 10% heat-inactivated horse serum at a density of 5650 cells/cm/cm onto 18 mm-diameter glass coverslips precoated with poly-L-lysine. Two-three h later, after neurons had adhered to the coverslips, they were transferred into 35 mm dishes containing 3 ml of cN2.1 medium (MEM with N2 supplements, conditioned by exposure to confluent cortical astrocytes for two days). Two coverslips with neurons were placed in each dish and arranged so that they did not overlap.

### LED Treatments:

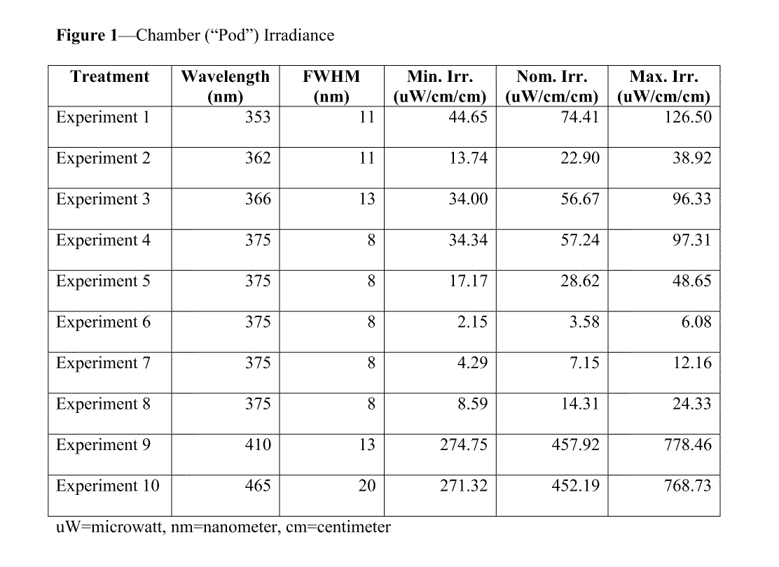

I. Irradiance which refers to the power of electromagnetic radiation depends on the wavelength of the emitted light. Typical wavelengths and irradiance of LED's used to study neuronal survival and differentiation in culture are shown in Table 2, wherein each wavelength range can correspond to one or more irradiance. According to one aspect of the invention, hippocampal neurons were exposed to LED treatment for 24 h beginning 3 h after plating by placing the culture dish inside a custom pod with an LED positioned in the top. Exposures were performed in two groups: 1) each LED emitted peak wavelength light of about 376 nm to about 377 nm, and a full width half maximum (“FWHM”) of about 8 nm, at different intensities that varied across a base 4 log range of about 8 nm to about 10 nm; 2) each LED emitted a different peak wavelengths between approximately 353 nm and 465 nm, with associated FWHM between 10 nm and 36 nm, with intensities ranging of about 2 microWatt/cm/cm to about 286 microWatt/cm/cm. Control cultures were either placed in a pod without an LED or were not in a pod. To prevent experimenter bias, all pods and dishes were labeled with codes and not with LED wavelength or intensity. The uncoded information wavelength and intensity information is provided in FIG. 1. Trays with pods and out-of-pod controls were placed in a humidified incubator at 36° C. and 5% CO2 for the duration of the treatments.

TABLE 2

| Wavelength of LED (nm) | Irradiance (uW/cm/cm) |
|---|---|
| 340-348 | 0.00-0.2 |
| 348-358 | 0.2-0.5 |
| 345-359 | 0.5-1.00 |
| 360-370 | 1.00-4.00 |
| 365-375 | 4.00-6.00 |
| 371-381 | 6.00-20.00 |
| 372-382 | 20.00-40.00 |
| 408-418 | 40.00-150.00 |
| 459-469 | 150.00-300.00 |

### Fixation and Immunofluorescent Cytochemistry.

Coverslips were removed from the dishes at the end of the LED treatment, rinsed briefly in warm PBS and fixed for 15 min in PBS containing 4% paraformaldehyde and 0.12M sucrose. Fixed cells were permeabilized in 0.3% Triton X-100 for 4 min. at room temperature, and rinsed in PBS. Some coverslips were stained to assess apoptosis (see below). After rinsing with PBS, coverslips were mounted in 1:1 PBS/Glycerol with 2.5% DABCO (anti-fade agent) and 0.1% sodium azide, and ringed with enamel. Slides were stored at 4° C. in the dark prior to analyses.

### Analysis of Neuron Survival:

To determine whether there was any cell loss associated with LED treatments, the survival of neurons was determined by comparing the mean number of pyramidal neurons per unit area of substrate using methods previously described in detail (Lindsley et al., 2002). Briefly, neurons from each treatment group were fixed, mounted on slides and the slides were coded to prevent experiementer bias. Cells were visualized by phase-contrast using a 10× objective, and the cell bodies (15-20 micrometers for pyramidal neurons, or 8-10 micrometers for interneurons) were counted in 15-35 microscope fields digitally captured from predetermined stage coordinates on 2 coverslips per treatment condition. Field boundaries were visualized using a 1 mm2 grid superimposed on the field of view in the microscope by placement of a reticle in the eyepiece. Only pyramidal neurons whose cell bodies were entirely located within the grid were counted as neurons, whereas non-pyramidal neurons and non-neuronal cells were counted separately. Pyramidal neurons are easily distinguished from non-pyramidal neurons and non-neuronal cells by their morphology: 1) a phase-dark, oval or pyramidal cell body; 2) a diameter of 15-20 μm; 3) numerous vacuoles, and 4) frequently 1 or 2 prominent nucleoli. Non-pyamidal interneurons have 8-10 μm diameter spindle-shaped cell bodies and 2-3 short, thick processes. Non-neuronal cells are flattened, have poor phase contrast and lack processes (Dotti et al., 1988). Changes in neuronal number over time in culture represent cell death only and are not confounded by proliferation of neurons because less than 1% of the neurons in these cultures divide (Dotti et al., 1988).

### Assessment of Apoptosis.

DNA fragmentation typically occurs in cells that are dying by an apoptotic mechanism (programmed cell death), and can be assessed using the TUNEL histological method [Gavrieli et al., 1992, J Cell Biol 119:493]. TUNEL (terminal deoxynucleotidyl transferase-mediated deoxyuridine triphosphate nick-end labeling) assays were performed using a commercially-available kit (Roche), according to the manufacturer's instructions. Proportion of cells stained with the TUNEL reagent was determined using the same unbiased methods described for analysis of neuronal survival. Positive controls (DNAase I treatment of fixed cells for 10 min) and negative controls (staining protocol carried out without TUNEL reagent) were used to confirm the specificity of staining

Quantitative Morphometric Analysis of Stage 1-3 Neurons Using Phase-Contrast Microscopy

Analysis of early stages of neuronal process outgrowth was performed essentially as previously described (Clamp and Lindsley, 1998). Briefly, for each replicate of an experiment, 2 coverslips of fixed cells from controls and 2 coverslips of each treatment group were analyzed. Coverslips were coded to avoid evaluator bias. Twenty fields per coverslip were chosen using pre-selected stage coordinates to ensure random, but even sampling of at least 400 cells per treatment. A grid, visually superimposed onto the fields using an eyepiece reticle, was used to delineate the boundaries of each field. Axon length was measured using the calibrated tracing tool in ImageJ software (NIH Image). Only neurons whose cell bodies were entirely located within the grid in each field were included in the analysis.

The criteria for developmental stages are as follows: Stage 1 neurons are defined by the presence of lamellipodia encircling the cell body and the absence of minor processes. Stage 2 neurons are defined by the presence of at least one minor process (typically about 15 μm long), with or without lamellipodia, and the absence of any process that exceeds the length equivalent of one grid unit (40 μm). Stage 3 neurons are defined by the presence of at least one process that can be identified unequivocally as an axon by its length being equal to or greater than one grid unit. These definitions represent a consensus of the nomenclature proposed by Dotti et al. (1988) and Goslin and Banker (1989). Parameters to be measured include the proportion of neurons in each stage of development, the number of minor processes per cell for stage 2 and stage 3 neurons, and the number of axons per stage 3 neuron.

Statistical Analyses. Results of cell counts, TUNEL staining and axon length measurements were calculated as Means±S.E.M., and analyzed using one-way ANOVA. Differences in proportion of cells in each stage of development, or proportion of cells showing a particular morphological feature were assessed using a Chi-Square test.

II. In an alternative embodiment rat hippocampal neurons in low-density primary cultures were exposed to light in the UVA1 range from a single LED in modular chambers (“pods”) constructed for this study by Opthera, Inc. Ten pods were provided; 5 emitted different peak wavelengths in the UVA1 range (˜340 nm to 400 nm), and 5 emitted the same peak wavelength, but at different power intensities. To ensure unbiased analyses, the wavelength and intensity of the pods were unknown to the investigators. Each pod was positioned over a dish containing neurons, then placed in an incubator at 36° C. and 5% CO2 that blocked room and outdoor light and maintained optimal temperature and pH during the experiment. The duration of LED exposure was 18-24 h. The timing of exposure was varied to determine whether sensitivity to the effects of UVA1 differed with stage of development.

The first set of experiments exposed neurons to the LEDs beginning 3 h after plating. Neurons at this stage of development are extending short processes that will later become the cell's axon and dendrites.

The second set of experiments exposed neurons to the LEDs beginning 1 day after plating, when 10-15% of the neurons have initiated axon growth (stage 3).

Control cultures were maintained in the same incubator and culture media but were not exposed to any LEDs. At the end of the period of LED exposure, we assessed the number of neurons and the rate at which neurons formed axons.

Neuronal cells used in this study were fixed and labeled as described above. The fixed cells are then analyzed for survival using phase contrast microscopy as described above.

### Summary of Results:

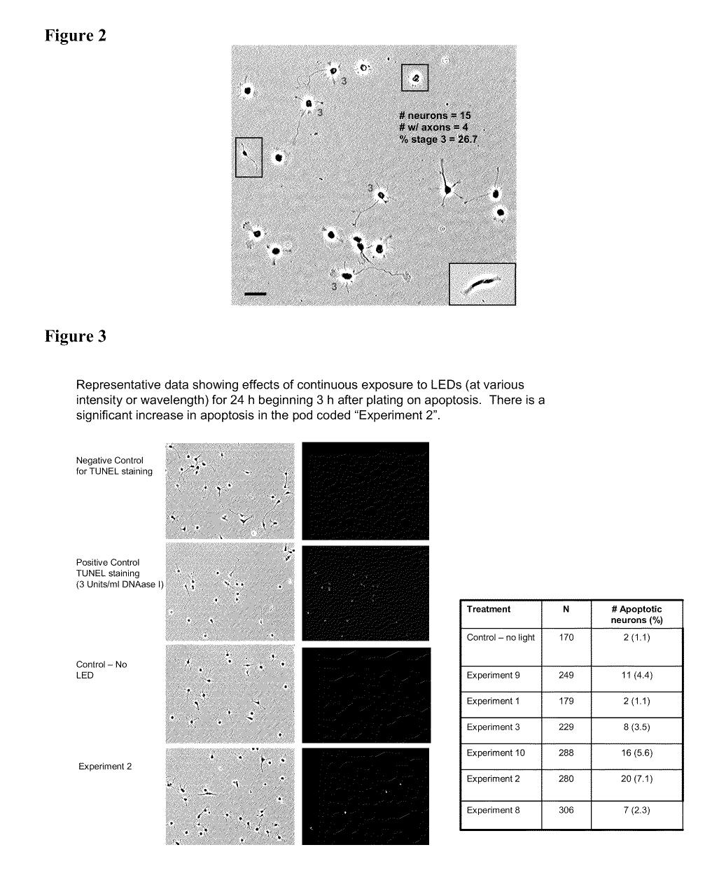

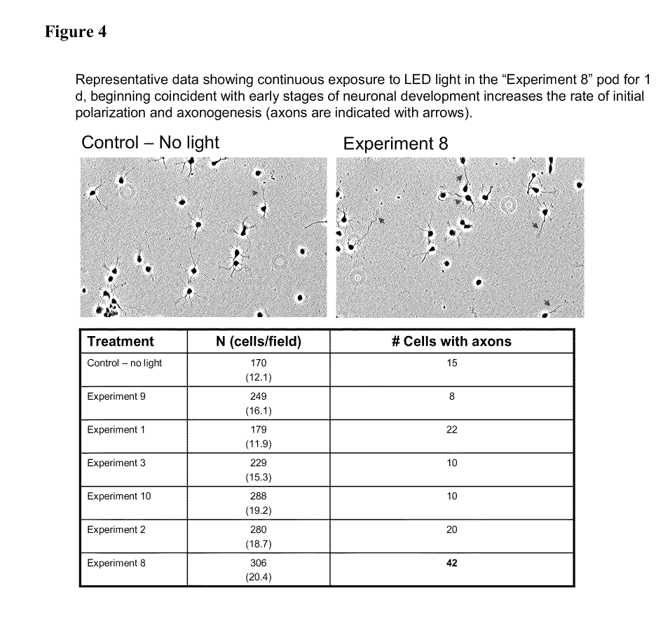

FIG. 2 shows a phase contrast micrograph of a representative field of cultured neuronal cells at different developmental stages one day after plating of neurons. The wavelength of light used in phototherapy affects neuronal survival. For example, as shown in FIG. 3 (p<0.004; Chi-Square), the number of hippocampal neurons with positive TUNEL staining was significantly higher for Experiment 2 pod, demonstrating that these experimental conditions promote apoptosis. In contrast, FIG. 4 shows no significant decrease in cell number per field for any treatment, demonstrating the LED treatments tested are not overtly cytotoxic to hippocampal neurons. All wavelengths (λ) are believed to be not cytotoxic.

Thus, hippocampal neurons have specific sensitivity to different wavelength and intensity of LED exposure. It is believed that the LED in Experiment 1 malfunctioned and that apoptosis would have been observed but for the LED malfunction. Thus, it is believed that radiation having peak wavelength of about 365 nm or lower, such as about 290 to about 365 nm, preferably about 350 nm to about 365 nm promotes apoptosis. Such radiation may have any suitable intensity, such as 1.8 microWatts/cm/cm or greater, for example 1.8 microWatts/cm/cm to 130 microWatts/cm/cm, including 2.2 microWatts/cm/cm to 17.7 microWatts/cm/cm.

The conditions of exposure in the Experiment 8 pod dramatically increased the rate at which hippocampal neurons initiate polarization and axonogenesis (FIG. 3, p<0.00001; Chi-Square of data from replicate experiments). Thus, radiation having peak wavelength of 370 nm or higher, such as 370 to 390 nm, preferably of about 376 nm to about 377 nm, promotes neuron morphogenesis (e.g., promotes neuron regeneration) without particular radiation intensity requirement. It also accelerates cell function with medium radiation intensity. Wavelength of about 410 nm to about 464 nm have similar effect but less effective. Preferably, the intensity is a medium intensity, such as 8 to about 25 microWatts/cm/cm.

As discussed in the prior section, the radiation may be provided to target cells, tissues, or organs by introducing into a body in need a radiation source in form of microparticle or nanoparticles, capsule, catheter, and functional equivalents. These radiation may also be applied to cells, such as blood cells outside the body for photophoresis treatment.

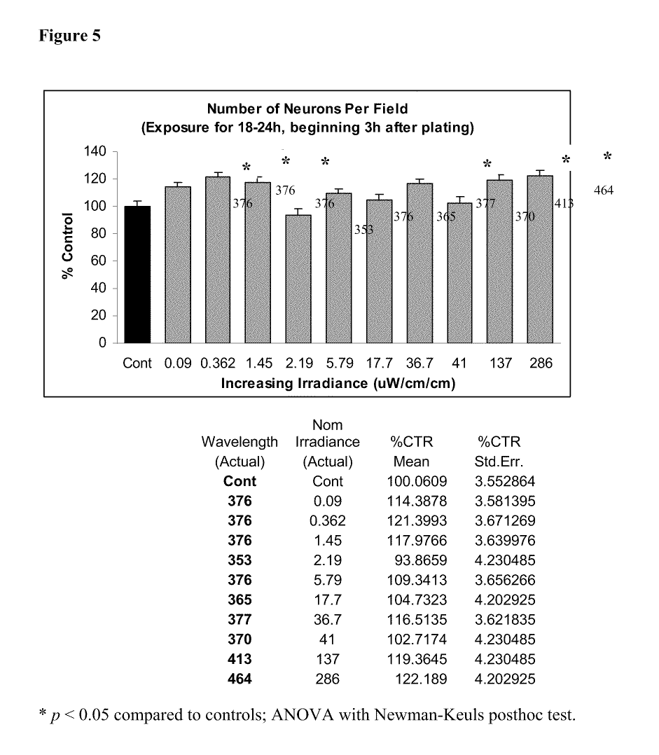

FIG. 5 shows that for hippocampal neurons exposed to UVA1 light in the range from about 340 nm to 400 nm, a significant increase in the number of neurons is observed after exposure to some, but not all wavelengths of UVA1 LEDs during the first 24 h in culture is most likely due to an effect of UVA1 that reduces cell death occurring over this time period. The 10-15% neuronal loss reported for these cultures (Clamp and Lindsley, 1998) is similar to the magnitude of LED-induced increase in neuron number. Alternatively, LED exposure at these wavelengths may increase neuronal proliferation, which is less than 1% under control conditions (Dotti et al., 1988). It is notable that a simple correlation between total energy of the session and this neuroprotective effect was not evident.

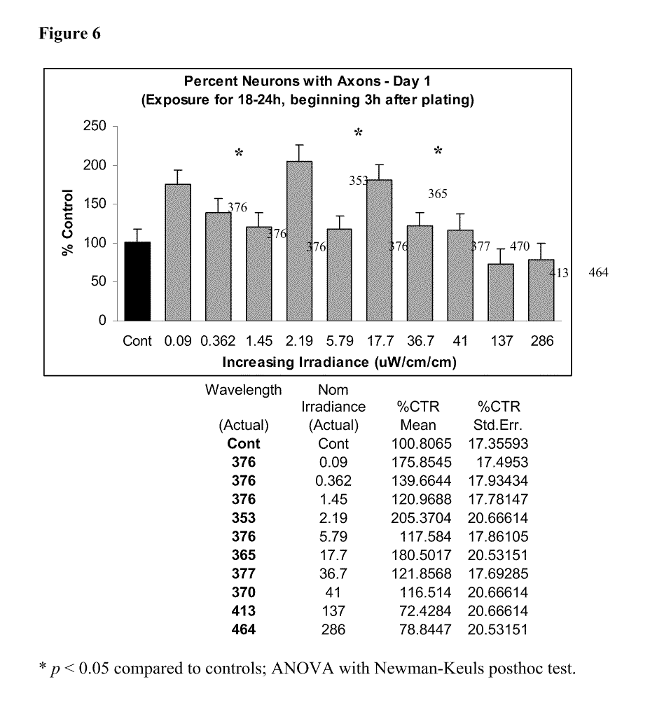

In fact, a 2-fold increase in percent of neurons with axons after exposure to the LED emitting at 353 nm (˜0.161 Joules/cm² for the session) during the first 24 h in culture (FIG. 6). These results suggest that certain conditions of UVA1 exposure can stimulate the rate of early stages of neuronal development, including axonogenesis. Interestingly, this occurred in only 3 Pods, only one of which also increased neuronal survival.

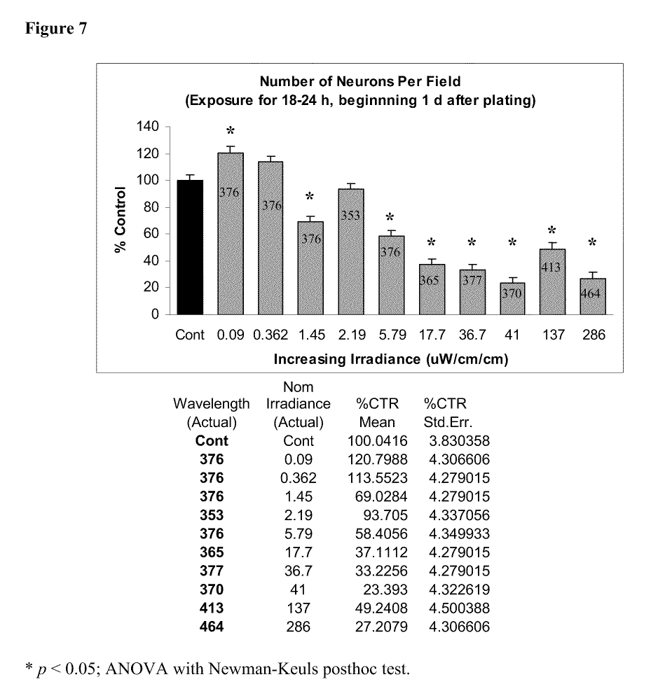

Furthermore, by delaying the start of UVA1 LED exposure for 1 day, a varied neuronal response to UVA1 was observed depending on the stage of development (FIG. 7). In contrast to the same duration, wavelengths and total energy effects after 18-24 h exposures beginning 3 h after plating, only one exposure condition (376 nm and ˜0.00782 J/cm² for the session) increased neuronal survival when the exposure was delayed for 1 day. All but the shortest wavelength and second lowest intensity of UVA1 exposure for 24 h was toxic during the period of rapid axon elongation.

Taken together, our results suggest that neuron survival and differentiation may be controlled by exposure to UVA1 light in a manner dependent on wavelength, intensity and timing relative to stage of differentiation. Importantly, the total energy density of UVA1 exposures in this study are about 4-times higher than what is typically delivered to human skin in clinical trials (McGrath, 1994; Szegedi, 2005; Pavel, 2006; Svobodova, 2006). UVA1 phototherapy to modulate neuronal survival and morphogenesis is a novel therapeutic approach for treating neurologic disease and injury that merits additional study.

The Following References are Incorporated by Reference in their Entirety:

Clamp P A, Lindsley T A. (1998) Early events in the development of neuronal polarity in vitro are altered by ethanol. Alcohol Clin Exp Res 22: 1277-1284.

Dotti C G, Sullivan C A, Banker G A. (1988) The establishment of polarity by hippocampal neurons in culture. J Neurosci 8(4):1454-68.

Fletcher T L, DeCamilli, P, G. Banker, (1994) Synaptogenesis in hippocampal cultures: Evidence indicating that axons and dendrites become competent to form synapses at different stages of neuronal development. J Neurosci 14:6695-6706.

Goslin K, Banker G. (1989) Experimental observations on the development of polarity by hippocampal neurons in culture. J Cell Biol 108(4):1507-16.

Goslin, K, Banker G (1991) Rat hippocampal neurons in low density culture. In: Culturing nerve cells. (Banker, G and Goslin, K eds.) MIT Press: Cambidge, Mass. pp. 251-282

Kaech S, Banker G (2006) Culturing hippocampal neurons. Nat Protocols, 1: 2406-2415.

Lindsley T A, Comstock L L, Rising L J. (2002) Morphologic and neurotoxic effects of ethanol vary with timing of exposure in vitro. Alcohol 28: 197-203.

Lindsley T A, Kerlin A, Rising L J. (2003) Time-lapse analysis of ethanol effects on axon growth in vitro. Dev Brain Res 147: 191-199.

Lindsley T A, Miller M W, Littner Y, Bearer C F. (2006) Signaling pathways regulating cell motility: A role in ethanol teratogenicity? Alcohol Clin Exp Res., 30: 1445-1450.

McGrath H. Jr. (1994) Ultraviolet-A1 irradiation decreases clinical disease activity and autoantibodies in patients with systemic lupus erythematosus. Clin Exp Rheumatol. 12:129-35.

Pavel S (2006) Light therapy (with UVA-1) for SLE patients: is it a good or bad idea? Rheumatology. 45:653-5.

Svobodova A, Walterova D, Vostalova J (2006) Ultraviolet light induced alteration to the skin. Biomed Pap Med Fac Univ Palacky Olomouc Czech Repub. 150:25-38.

Yanni P A and Lindsley T A. (2000) Ethanol inhibits development of dendrites and synapses in hippocampal pyramidal neuron cultures. Dev Brain Res 120: 233-243.

What is claimed is:

1. A phototherapy method comprising providing ultraviolet light to at least one of:
(a) below a skin of a mammal; or
(b) to a body cavity, lumen, organ, tissue or tissue within the cavity of the mammal; or
(c) within a blood or to the blood or to a specific type of cell of the mammal;
to provide a phototherapeutic effect thereto.

2. The method of claim 1, wherein the ultraviolet light comprises UVA1 light.

3. The method of claim 2, wherein the ultraviolet light comprises UVA1C light.

4. A phototherapy device comprising a UV-LED which in operation provides ultraviolet light to at least one of:
(a) below a skin of a mammal; or
(b) to a body cavity, lumen, organ, tissue or tissue within the cavity of the mammal; or
(c) within a blood or to the blood or to a specific type of cell of the mammal; to provide a phototherapeutic effect thereto.

5. A method, comprising providing ultraviolet light to a mammal to alter a population or a ratio of cells in the mammal to provide at least one therapeutic effect to the mammal.

6. The method of claim 5, wherein said ratio of cells in the mammal is the ratio of T*SUB*H1 cells to T*SUB*H2 cells.

7. A phototherapy method comprising providing LED radiation to at least one of:
(a) below a skin of a mammal; or
(b) to a body cavity, lumen, organ, tissue or tissue within the cavity of the mammal; or
(c) within a blood or to the blood or to a specific type of cell of the mammal;
to provide a phototherapeutic effect thereto.

8. The method of claim 7, wherein the radiation is provided to hippocampal neurons.

9. The method of claim 8, wherein the radiation comprises a peak wavelength of about 362 nm or lower and the phototherapeutic effect is promoting apoptosis.

10. The method of claim 7, wherein the radiation comprises a peak wavelength of about 370 nm or higher.

11. The method of claim 10, wherein the radiation comprises peak wavelength of about 375 nm and an intensity of about 8 to about 25 microWatts/cm², and the phototherapeutic effect is promoting hippocampal neuron morphogenesis.

12. A phototherapy method comprising providing radiation to neurons to promote apoptosis or promote neuron morphogenesis.

13. The method of claim 12, wherein the radiation comprises a peak wavelength of about 370 nm or higher to promote neuron morphogenesis.

14. The method of claim 13, wherein the radiation comprises a peak wavelength of about 375 nm and an intensity of about 8 to about 25 microWatts/cm².

15. The method of claim 12, wherein the radiation comprises a peak wavelength of about 365 nm or lower to promote apoptosis.

16. The method of claim 12, wherein the neurons comprise hippocampal neurons.

17. The method of claim 12, wherein the radiation comprises UV radiation from a LED source.
